(19) United States
(12) Patent Application Publication
(10) Pub. No.: US 2011/0312516 A1
(43) Pub. Date: Dec. 22, 2011

(54) DIAGNOSTIC AND PROGNOSTIC USE OF HUMAN BLADDER CANCER-ASSOCIATED MICRO RNAS

(75) Inventors: Torben F. Orntoft, Silkeborg (DK); Lars D. Andersen, Odder (DK)

(73) Assignee: Catalyst Assets LLC, Jackson, WY (US)

(21) Appl. No.: 13/052,038

(22) Filed: Mar. 18, 2011

Related U.S. Application Data
(63) Continuation of application No. PCT/US08/09861, filed on Aug. 18, 2008.
(60) Provisional application No. 60/965,185, filed on Aug. 17, 2007.

(51) Int. Cl. C40B 30/04; C12Q 1/68

(52) U.S. Cl. 506/9; 435/6.12; 435/6.11

(57) ABSTRACT

The present invention is based, at least in part, upon discovery of a number of miRNAs having expression that significantly correlates with bladder cancer, including certain stages or types of bladder cancer, as well as with bladder cancer survival and/or responsiveness to bladder cancer therapies. Accordingly, the present invention features the identification and use of miRNAs to detect, diagnose and/or predict the course, progression or therapy responsiveness of bladder cancer. Kits for performing such assessments, and for administering therapeutic agents to subjects diagnosed with bladder cancer or certain forms of bladder cancer using the methods of the invention, are also featured.

### RELATED APPLICATIONS

This application is a continuation of PCT International application No. PCT/US08/009,861, filed Aug. 18, 2008, which claims the benefit of U.S. Provisional patent application No. 60/965,185, filed Aug. 17, 2007. The entire contents of each of these applications are incorporated herein by reference.

### BACKGROUND OF THE INVENTION

Predicting onset and disease progression for patients with non-muscle invasive tumors is difficult and error prone. To date no molecular markers for predicting progression are used in clinical routine.

### SUMMARY OF THE INVENTION

The present invention is based, at least in part, upon discovery of a number of miRNAs having expression that significantly correlates with bladder cancer, including certain stages or types of bladder cancer, as well as with bladder cancer survival and/or responsiveness to bladder cancer therapies. Accordingly, the present invention features the identification and use of miRNAs to detect, diagnose and/or predict the course, progression or therapy responsiveness of bladder cancer. Kits for performing such assessments, and for administering therapeutic agents to subjects diagnosed with bladder cancer or certain forms of bladder cancer using the methods of the invention, are also featured. Other features and advantages of the invention will be apparent from the detailed description, and from the claims.

In one aspect, the invention provides a method for identifying bladder cancer in a tissue sample involving collecting a tissue sample from the subject, determining an expression pattern in the sample for a plurality of miRNAs comprising miRNAs listed in Tables 3-5 and/or FIGS. 1A-C, FIGS. 5A-5C and/or FIG. 8, and correlating the expression pattern of this plurality of miRNAs to a standard expression pattern for this plurality of miRNAs to identify the presence of bladder cancer in the tissue sample.

In one embodiment, the tissue comprises bladder tissue. In another embodiment, the plurality of miRNAs comprises at least two miRNAs selected from Table 3. In a further embodiment, the plurality of miRNAs comprises at least two miRNAs selected from FIG. 1A. In a related embodiment, the plurality of miRNAs comprises at least one of miR-193a, miR-519e*, miR-21, miR-202, miR-198, miR-145, miR-455, miR-143, miR-125b and miR-126.

In another embodiment, the steps of determining an expression pattern and correlating the expression pattern for the tissue sample to that of a standard expression pattern involve determining a score corresponding to the expression pattern of the tissue sample and comparing this sample score to a score determined for the standard expression pattern.

In an additional embodiment, the standard expression pattern includes the expression pattern of the plurality of miRNAs in a control bladder cancer tissue. In a related embodiment, the stage of the control bladder cancer tissue is T2, T3 or T4. In another embodiment, the standard expression pattern includes the expression pattern of the plurality of miRNAs in normal urothelium tissue. In a further embodiment, elevation in the sample of a plurality of miRNAs comprising miRNAs selected from miR-193a, miR-519e*, miR-21, miR-202 and miR-198, relative to the expression levels of the plurality of miRNAs in the standard expression pattern identifies the presence of bladder cancer in the subject. In another embodiment, reduction in the sample of a plurality of miRNAs comprising miRNAs selected from miR-145, miR-455, miR-143, miR-125b and miR-126, relative to the expression levels of the plurality of miRNAs in the standard expression pattern identifies the presence of bladder cancer in the subject. In an additional embodiment, elevation in the sample of at least one miRNA selected from miR-193a, miR-519e*, miR-21, miR-202 and miR-198 relative to the expression level of the at least one miRNA in the standard expression pattern and reduction in the sample of at least one other miRNA selected from miR-145, miR-455, miR-143, miR-125b and miR-126 relative to the expression level of the at least one other miRNA in the standard expression pattern identifies the presence of bladder cancer in the subject. In a further embodiment, the standard expression pattern comprises the mean expression pattern of the plurality of miRNAs measured in at least two control bladder cancer samples. In a related embodiment, the at least two control bladder cancer samples are of a single bladder stage that is Ta, T1, T2, T3 or T4. In another embodiment, the at least two control bladder cancer samples are of at least two different bladder cancer stages selected from Ta, T1, T2, T3 and T4.

In another embodiment, the plurality of miRNAs comprises at least two miRNAs selected from Table 4. In a further embodiment, the plurality of miRNAs comprises at least two miRNAs selected from FIG. 5A. In a related embodiment, the plurality of miRNAs comprises at least one of miR-519e*, miR-193a-3p, miR-21, miR-20a, miR-198, miR-510, miR-184, miR-492, miR-455-5p, miR-143, miR-145, miR-126*, miR-26a, miR-125b, miR-498, miR-489, miR-503, miR-29a, miR-302b and miR-29c.

In a further embodiment, elevation in the sample of a plurality of miRNAs comprising miRNAs selected from miR-519e*, miR-193a-3p, miR-21, miR-20a, miR-198, miR-510, miR-184 and miR-492, relative to the expression levels of the plurality of miRNAs in the standard expression pattern identifies the presence of bladder cancer in the subject. In another embodiment, reduction in the sample of a plurality of miRNAs comprising miRNAs selected from miR-455-5p, miR-143, miR-145, miR-126*, miR-26a, miR-125b, miR-498, miR-489, miR-503, miR-29a, miR-302b and miR-29c, relative to the expression levels of the plurality of miRNAs in the standard expression pattern identifies the presence of bladder cancer in the subject. In an additional embodiment, elevation in the sample of at least one miRNA selected from miR-519e*, miR-193a-3p, miR-21, miR-20a, miR-198, miR-510, miR-184 and miR-492 relative to the expression level of the at least one miRNA in the standard expression pattern and reduction in the sample of at least one other miRNA selected from miR-455-5p, miR-143, miR-145, miR-126*, miR-26a, miR-125b, miR-498, miR-489, miR-503, miR-29a, miR-302b and miR-29c relative to the expression level of the at least one other miRNA in the standard expression pattern identifies the presence of bladder cancer in the subject.

In another embodiment, the step of determining involves detecting the expression level of at least one of the miRNAs using quantitative polymerase chain reaction (QPCR). In an additional embodiment, the step of determining involves detecting the expression level of at least one of the miRNAs using in situ hybridization.

In another aspect, the invention provides a method for predicting the prognosis of bladder cancer in a subject involving collecting a sample comprising bladder cancer cells from the subject; determining an expression pattern of a plurality of miRNAs in the sample, where the plurality of miRNAs include at least two miRNAs selected from those miRNAs listed in Tables 3 and 5 and FIGS. 1B and 5B; and correlating the expression pattern of this plurality of miRNAs to at least one standard expression pattern of this plurality of miRNAs to predict the prognosis of the bladder cancer in the subject.

In an additional aspect, the invention provides a method for predicting the likelihood of bladder cancer progression in a subject involving collecting a sample comprising bladder cancer cells from the subject; determining an expression pattern of a plurality of miRNAs in the sample, where the plurality of miRNAs includes at least two miRNAs selected from the miRNAs listed in Tables 3 and 5 and FIGS. 1B and 5B; and correlating the expression pattern of this plurality of miRNAs to at least one standard expression pattern of this plurality of miRNAs to predict the likelihood of bladder cancer progression in the subject.

In one embodiment, the bladder cancer cells are Ta stage bladder cancer cells. In another embodiment, the bladder cancer cells are of at least one stage of Ta and T1. In an additional embodiment, the plurality of miRNAs includes at least one miRNA selected from miR-483, miR-526c, miR-503, miR-129, miR-320, miR-145 and miR-133b. In another embodiment, the standard expression pattern includes an expression pattern of the plurality of miRNAs in a Ta or T1 stage bladder cancer that has subsequently progressed. In a further embodiment, the standard expression pattern includes an expression pattern of the plurality of miRNAs in a Ta or T1 stage bladder cancer that has subsequently not progressed. In another embodiment, the bladder cancer cells are of at least one stage of Ta and T1, and the plurality of miRNAs consists of two miRNAs. Optionally, these two miRNAs are miR-129 and miR-133b. In an additional embodiment, the bladder cancer cells are Ta stage bladder cancer cells, and the plurality of miRNAs consists of four miRNAs. Optionally, these four miRNAs are miR-129, miR-133b and miR-320.

In another embodiment, the plurality of miRNAs includes at least one miRNA of miR-129, miR-133b, miR-320, miR-503, miR-373*, miR-526c, miR-498, miR-518c*, miR-185, miR-483, miR-145; miR-29c, miR-29b, miR-29a and miR-203. In an additional embodiment, elevation in the sample of a plurality of miRNAs comprising at least two miRNAs of miR-129, miR-133b, miR-320, miR-503, miR-373*, miR-526c, miR-498, miR-518c*, miR-185, miR-483 and miR-145 relative to the standard expression pattern of the plurality of miRNAs identifies bladder cancer that is likely to progress in the subject. In another embodiment, reduction in the sample of a plurality of miRNAs including at least two miRNAs of miR-29c, miR-29b, miR-29a and miR-203 relative to the standard expression pattern of the plurality of miRNAs identifies bladder cancer that is likely to progress in the subject. In an additional embodiment, elevation in the sample of at least one miRNA of miR-129, miR-133b, miR-320, miR-503, miR-373*, miR-526c, miR-498, miR-518c*, miR-185, miR-483 and miR-145 and reduction in the sample of at least one miRNA of miR-29c, miR-29b, miR-29a and miR-203 relative to the standard expression pattern level of these selected miRNAs identifies bladder cancer that is likely to progress in the subject.

In an additional embodiment, the plurality of miRNAs includes at least one miRNA selected from miR-129-5p, miR-518c*, miR-185, miR-133b, miR-373*, miR-320, miR-29e, miR-29b, miR-29a, miR-36′-5p, miR-203 and miR-205. In another embodiment, elevation in the sample of a plurality of miRNAs comprising at least two miRNAs of miR-129-5p, miR-518c*, miR-185, miR-133b, miR-373*, miR-320 and miR-145 relative to the standard expression pattern of the plurality of miRNAs identifies bladder cancer that is likely to progress in the subject. In another embodiment, reduction in the sample of a plurality of miRNAs including at least two miRNAs of miR-29c, miR-29b, miR-29a, miR-361-5p, miR-203 and miR-205 relative to the standard expression pattern of the plurality of miRNAs identifies bladder cancer that is likely to progress in the subject. In an additional embodiment, elevation in the sample of at least one miRNA of miR-129-5p, miR-518c*, miR-185, miR-133b, miR-373*, miR-320 and miR-145 and reduction in the sample of at least one miRNA of miR-29c, miR-29b, miR-29a, miR-361-5p, miR-203 and miR-205 relative to the standard expression pattern level of these selected miRNAs identifies bladder cancer that is likely to progress in the subject.

In another embodiment, the plurality of miRNAs includes at least one miRNA of miR-129 and miR-503. In an additional embodiment, the standard expression pattern includes an expression pattern of the plurality of miRNAs in bladder cancer that is classified as carcinoma-in-situ (CIS).

In another aspect, the invention provides a method for identifying an advanced stage bladder cancer sample involving obtaining a sample comprising bladder cancer cells; determining an expression pattern of a plurality of miRNAs in the sample, where the plurality of miRNAs includes miRNAs selected from the miRNAs listed in Tables 3 and 4 and FIGS. 1A and 5A; and correlating the expression pattern of this plurality of miRNAs to at least one standard expression pattern of this plurality of miRNAs to identify an advanced stage bladder cancer sample.

In one embodiment, the plurality of miRNAs includes at least one miRNA selected of miR-129, miR-503, miR-320, miR-498, miR-483, let-7g, miR-18b, miR-200b, miR-141 and miR-219. In another embodiment, the standard expression pattern includes an expression pattern of the plurality of miRNAs in bladder cancer of stage T2, T3 or T4. In a further embodiment, the standard expression pattern includes an expression pattern of the plurality of miRNAs in normal urothelium tissue. In an additional embodiment, the elevation in the sample of a plurality of miRNAs including miRNAs selected from miR-129, miR-503, miR-320, miR-498 and miR-483 relative to the standard expression pattern of the plurality of miRNAs identifies an advanced stage bladder cancer sample. In another embodiment, reduction in the sample of a plurality of miRNAs intending miRNAs selected from let-7g, miR-18b, miR-200b, miR-141 and miR-219 relative to the standard expression pattern of the plurality of miRNAs identifies an advanced stage bladder cancer sample. In a further embodiment, elevation in the sample of at least one of miR-129, miR-503, miR-320, miR-498 and miR-483 and reduction in the sample of at least one of let-7g, miR-18b, miR-200b, miR-141 and miR-219 relative to the standard expression pattern level of these miRNAs identifies an advanced stage bladder cancer sample.

In another embodiment, the plurality of miRNAs includes at least one of miR-129 and miR-503.

In one embodiment, the plurality of miRNAs includes at least one miRNA selected from miR-503, miR-129-5p, miR-320a, miR-193b, miR-483-3p, miR-498, miR-516b, miR-328, miR-373*, miR-519e*, miR-526b, miR-296-5p, miR-490-3p, miR-489, miR-18b, miR-219-5p, miR-98, miR-141, miR-200c, miR-191*, miR-203 and miR-34b. In an additional embodiment, the elevation in the sample of a plurality of miRNAs including miRNAs selected from miR-503, miR-129-5p, miR-320a, miR-193b, miR-483-3p, miR-498, miR-516b, miR-328, miR-373*, miR-519e*, miR-526b, miR-296-5p, miR-490-3p and miR-489 relative to the standard expression pattern of the plurality of miRNAs identifies an advanced stage bladder cancer sample. In another embodiment, reduction in the sample of a plurality of miRNAs including miRNAs selected from miR-18b, miR-219-5p, miR-98, miR-141, miR-200c, miR-191*, miR-203 and miR-34b relative to the standard expression pattern of the plurality of miRNAs identifies an advanced stage bladder cancer sample. In a further embodiment, elevation in the sample of at least one of miR-503, miR-129-5p, miR-320a, miR-193b, miR-483-3p, miR-498, miR-516b, miR-328, miR-373*, miR-519e*, miR-526b, miR-296-5p, miR-490-3p and miR-489 and reduction in the sample of at least one of miR-18b, miR-219-5p, miR-98, miR-141, miR-200c, miR-191*, miR-203 and miR-34b relative to the standard expression pattern level of these miRNAs identifies an advanced stage bladder cancer sample.

In another aspect, the invention provides a method for detecting carcinoma-in-situ in a bladder tissue sample involving collecting a bladder tissue sample from a subject; determining an expression pattern of a plurality of miRNAs in the sample, where the miRNAs are selected from those listed in Tables 3 and 5 and FIGS. 1C and 5C; and correlating the expression pattern of this plurality of miRNAs to at least one standard expression pattern of this plurality of miRNAs to identify carcinoma-in-situ in the bladder tissue sample.

In one embodiment, the plurality of miRNAs includes miRNAs selected from miR-129, miR-503, miR-498, miR-483, miR-451, miR-373*, miR-451, miR-320, miR-205, miR-382, miR-200a*, miR-487a, miR-191*, miR-29c, miR-210, miR-302b, miR-324-5p, miR-127, miR-31, miR-345 and miR-202*. In another embodiment, the standard expression pattern includes an expression pattern of the plurality of miRNAs in bladder cancer of stage Ta or T1, where the standard bladder cancer is classified as carcinoma-in-situ (CIS). In an further embodiment, the standard expression pattern includes an expression pattern of the plurality of miRNAs in bladder cancer of stage Ta or T1, where the standard bladder cancer is not classified as carcinoma-in-situ (CIS).

In another embodiment, elevation in the sample of a plurality of miRNAs including at least one miRNA selected from miR-129, miR-503, miR-498, miR-483, miR-451, miR-373*, miR-451 and miR-320 relative to the standard expression pattern of this plurality of miRNAs identifies carcinoma-in-situ in the bladder tissue sample. In an additional embodiment, reduction in the sample of a plurality of miRNAs including at least one miRNA of miR-205, miR-382, miR-200a*, miR-487a, miR-191*, miR-29c, miR-210, miR-302b, miR-324-5p, miR-127, miR-31, miR-345 and miR-202* in the sample relative to the standard expression pattern of this plurality of miRNAs identifies carcinoma-in-situ in the bladder tissue sample. In a further embodiment, elevation in the sample of at least one miRNA of miR-129, miR-503, miR-498, miR-483, miR-451, miR-373*, miR-451 and miR-320 relative to the standard expression pattern level of the at least one miRNA and reduction in the sample of at least one other miRNA of miR-205, miR-382, miR-200a*, miR-487a, miR-191*, miR-29c, miR-210, miR-302b, miR-324-5p, miR-127, miR-31, miR-345 and miR-202* relative to the standard expression pattern level of the at least one other miRNA identifies carcinoma-in-situ in the bladder tissue sample. In another embodiment, an expression pattern is determined for a plurality of miRNAs selected from among those miRNAs listed in FIG. 1C. In an additional embodiment, an expression pattern is determined for a plurality of miRNAs comprising at least one miRNA selected from the group consisting of miR-129, miR-146, miR-17-5p and miR-503. In a further embodiment, an expression pattern is determined for ten miRNAs. Optionally, these ten miRNAs include miR-129, miR-191*, miR-200a*, miR-205, miR-382, miR-487a, miR-498 and miR-503.

In another embodiment, the standard expression pattern includes an expression pattern of the plurality of miRNAs in a T1 bladder cancer sample, where the standard bladder cancer is not classified as carcinoma-in-situ (CIS). In a further embodiment, the standard expression pattern includes an expression pattern of the plurality of miRNAs in a T1 bladder cancer sample, where the standard bladder cancer is classified as carcinoma-in-situ (CIS).

In an additional embodiment, an expression pattern is determined for a plurality of miRNAs selected from those listed in FIG. 1C. In a further embodiment, an expression pattern is determined for seven miRNAs. Optionally, these seven miRNAs include miR-146a, miR-17-5p and miR-210. In an additional embodiment, the plurality of miRNAs includes at least one of miR-129 and miR-503. Optionally, the plurality of miRNAs includes both miR-129 and miR-503.

In a further embodiment, the standard expression pattern includes an expression pattern of the plurality of miRNAs in normal urothelium tissue. In one embodiment, the bladder tissue sample comprises a bladder cancer tumor. In a related embodiment, the bladder cancer is of stage Ta or T1. In another embodiment, the bladder cancer is a T1 bladder cancer. In another embodiment, the plurality of miRNAs includes miR-129 and miR-503.

In one embodiment, the plurality of miRNAs includes miRNAs selected from miR-503, miR-498, miR-129-5p, miR-373*, miR-483-3p, miR-320, miR-451, miR-19a, miR-205, miR-191*, miR-382, miR-127-3p, miR-29c, miR-200a*, miR-31, miR-302b, miR-345 and miR-87a. In another embodiment, elevation in the sample of a plurality of miRNAs including at least one miRNA selected from miR-503, miR-498, miR-129-5p, miR-373*, miR-483-3p, miR-320, miR-451 and miR-19a relative to the standard expression pattern of this plurality of miRNAs identifies carcinoma-in-situ in the bladder tissue sample. In an additional embodiment, reduction in the sample of a plurality of miRNAs including at least one miRNA of miR-205, miR-191*, miR-382, miR-127-3p, miR-29c, miR-200a*, miR-31, miR-302b, miR-345 and miR-87a in the sample relative to the standard expression pattern of this plurality of miRNAs identifies carcinoma-in-situ in the bladder tissue sample. In a further embodiment, elevation in the sample of at least one miRNA of miR-503, miR-498, miR-129-5p, miR-373*, miR-483-3p, miR-320, miR-451 and miR-19a relative to the standard expression pattern level of the at least one miRNA and reduction in the sample of at least one other miRNA of miR-205, miR-191*, miR-382, miR-127-3p, miR-29c, miR-200a*, miR-31, miR-302b, miR-345 and miR-87a relative to the standard expression pattern level of the at least one other miRNA identifies carcinoma-in-situ in the bladder tissue sample.

In an additional aspect, the invention provides a method for identifying bladder cancer in a tissue sample involving collecting a tissue sample from a subject; determining an expression level for a miRNA in the sample, where the miRNA is selected from among those listed in Table 3, Table 4, Table 5, FIG. 1A, FIG. 1B, FIG. 1C, FIG. 5A, FIG. 5B, FIG. 5C and FIG. 8; and comparing the expression level of the miRNA to the expression level of the miRNA in a control sample to identify the presence of bladder cancer in the tissue sample.

In another aspect, the invention provides a method for identifying a subject in need of cancer therapy involving collecting a test sample comprising bladder tissue from the subject; determining an expression level of miR-129 in the test sample; and comparing the expression level of miR-129 in the test sample to the expression level of miR-129 in a control sample to identify a subject in need of cancer therapy.

In one embodiment, an elevated miR-129 expression level in the test sample relative to the control sample identifies a subject in need of cancer therapy. In another embodiment, the expression level of miR-129 in the control sample is a median expression level of miR-129 in a plurality of control samples. In a further embodiment, a miR-129 expression level that is elevated at least two-fold in the test sample relative to the control sample identifies a subject in need of cancer therapy. Optionally, the threshold for identification of a subject in need of cancer therapy is a 2.2-fold elevation of miR-129 expression. In another embodiment, the control sample is T2, T3 or T4 stage bladder cancer. In a further embodiment, a similar miR-129 expression level in the test sample relative to the control sample identifies a subject in need of cancer therapy. In an additional embodiment, the method is performed after the subject is administered chemotherapy.

An additional aspect of the invention provides a method for identifying a subject in need of cancer therapy involving collecting a test sample comprising bladder tissue from the subject; determining an expression level of miR-29c in the test sample; and comparing the expression level of miR-29c in the test sample to the expression level of miR-29c in a control sample to identify a subject in need of cancer therapy.

In one embodiment, a decreased miR-29c expression level in the test sample relative to the control sample identifies a subject in need of cancer therapy. In another embodiment, a similar miR-29c expression level in the test sample relative to the control sample identifies a subject in need of cancer therapy. In a further embodiment, the method is performed after the subject is administered chemotherapy.

An additional aspect of the invention provides a kit for measuring the level of a miRNA that includes at least one oligonucleotide that specifically binds to at least one miRNA listed in FIG. 1A, FIG. 1B, FIG. 1C, FIG. 5A, FIG. 5B, FIG. 5C, Table 3, Table 4 and Table 5, with the kit also including instructions for its use.

In one embodiment, the kit also comprises a nonspecific oligonucleotide for amplification of a miRNA.

Another aspect of the invention provides an oligonucleotide microarray having immobilized thereon a plurality of oligonucleotide probes specific for one or more miRNAs listed in FIG. 1A, FIG. 1B, FIG. 1C, FIG. 5A, FIG. 5B, FIG. 5C, Table 3, Table 4 and Table 5.

In another aspect, the invention provides a method for detecting an FGF3 mutant tumor in a bladder tissue sample involving collecting a bladder tissue sample from a subject; determining an expression pattern of a plurality of miRNAs in the sample, where the miRNAs are selected from those listed in Table 5 and FIG. 5C; and correlating the expression pattern of this plurality of miRNAs to at least one standard expression pattern of this plurality of miRNAs to identify an FGF3 mutant tumor in the bladder tissue sample.

In one embodiment, the plurality of miRNAs includes miRNAs selected from miR-498, miR-129-5p, miR-145, miR-148b, miR-10b and miR-324-5p. In another embodiment, elevation in the sample of a plurality of miRNAs including at least one miRNA selected from miR-498, miR-129-5p and miR-145 relative to the standard expression pattern of this plurality of miRNAs identifies an FGF3 mutant tumor in the bladder tissue sample. In an additional embodiment, reduction in the sample of a plurality of miRNAs including at least one miRNA of miR-148b, miR-10b and miR-324-5p in the sample relative to the standard expression pattern of this plurality of miRNAs identifies an FGF3 mutant tumor in the bladder tissue sample. In a further embodiment, elevation in the sample of at least one miRNA of miR-498, miR-129-5p and miR-145 relative to the standard expression pattern level of the at least one miRNA and reduction in the sample of at least one other miRNA of miR-148b, miR-10b and miR-324-5p relative to the standard expression pattern level of the at least one other miRNA identifies an FGF3 mutant tumor in the bladder tissue sample.

### BRIEF DESCRIPTION OF THE DRAWINGS

FIGS. 1A-C show expression patterns of miRNAs found to be significantly differentially expressed in comparisons of bladder cancers (of varying stages) and normal tissues. Marker-sets A-C were clustered hierarchically in order to align similar profiles. Median expression of miRNA is indicated in black. Those miRNA expression levels that were also validated by quantitative PCR are marked with “QPCR” in the gene name lists. FIG. 1A shows the expression of 80 miRNA identified to be significantly regulated between normal, Ta, T1 and T2-4 tumors.

FIG. 1B shows the expression patterns of 15 miRNA identified as significantly differentially expressed between non-progressing and progressing tumors, wherein miRNAs highlighted in bold represent those miRNAs that showed significant expression level differences when T1 tumors only were compared. The top bars indicate the group labels (non-progressing, progressing, Ta, and T1), while the bottom bar indicates the CIS status (black: CIS, white: no CIS, gray: ND). FIG. 1C shows expression patterns of the 20 miRNA identified as significantly differentially expressed between no-CIS and CIS patients, where bold miRNA names indicate miRNAs that showed significant differences in expression level when T1 tumors only were analyzed. The bottom bar indicates those tumors showing subsequent (later) progression to T2-4.

FIG. 2A shows a comparison of miRNA expression data from quantitative PCR

(QPCR) and microarray measurements. FIG. 2B depicts a Kaplan Meier plot of progression-free survival as a function of the expression of miR-129 (medium expression of miR-129 was used for grouping the samples according to high and low expression).

FIG. 3 shows in situ hybridization localization of miRNA expression in tissue sections and haematoxylin-eosin (HE) stained overview sections of tumor tissues. Images are overlay images with DAPI staining present and brightest staining representing miRNA expression (except for miR-145 tumor image that only include the miRNA expression). The sections for in situ hybridization and for HE staining are consecutive sections—not identical sections.

FIGS. 4A-D show charts corresponding to classification of bladder cancer-related phenotypes using single miRNA expression level values in a collection of samples. FIG. 4A shows classification of normal versus cancer samples using miR-193a expression levels to define a cutoff value for classification. FIG. 4B shows classification of Ta versus T2 stage bladder cancer samples using miR-129 expression levels to define a cutoff value for classification. FIG. 4C shows classification of no CIS versus CIS bladder cancer samples using miR-503 expression levels to define a cutoff value for classification. FIG. 4D shows classification of no progression versus progression samples using miR-129 expression levels to define a cutoff value for classification.

FIGS. 5A-C show expression patterns of miRNAs found to be significantly differentially expressed in comparisons of bladder cancers (of varying stages) and normal tissues. Marker-sets A-C were clustered hierarchically in order to align similar profiles. Median expression of miRNA is indicated in black. Those miRNA expression levels that were also validated by quantitative PCR are marked with “QPCR” in the gene name lists. FIG. 5A shows the expression of 80 miRNA identified to be significantly regulated between normal, Ta, T1 and T2-4 tumors. FIG. 5B shows the expression patterns of 15 miRNA identified as significantly differentially expressed between non-progressing and progressing tumors, wherein miRNAs highlighted in bold represent those miRNAs that showed significant expression level differences when T1 tumors only were compared. The top bars indicate the group labels (non-progressing, progressing, Ta, and T1), while the bottom bar indicates the CIS status (black: CIS, white: no CIS, gray: ND). FIG. 5C shows expression patterns of the 20 miRNA identified as significantly differentially expressed between no-CIS and CIS patients, where bold miRNA names indicate miRNAs that showed significant differences in expression level when T1 tumors only were analyzed. The bottom bar indicates those tumors showing subsequent (later) progression to T2-4.

FIG. 6 shows Kaplan-Meier plots of progression-free survival as a function of the expression of miR-133b, miR-129, miR-29c, miR-518c*, miR-133b+miR-518c* and miR-133b+miR-129. Optimal cutoff points were determined for each miRNA using ROC curves in order to categorize the expressions into low and high groups. Log-rank test results are shown for each Kaplan-Meier plot.

FIG. 7 shows comparisons of miRNA expression data from QPCR and microarray measurements.

FIG. 8 shows hierarchical cluster analysis of the 120 probes detected above the background filter in more than 25% of samples. The tumor stage is shown below the cluster dendrogram: normal: green, Ta: yellow, T1: orange, T2-4: red, cell lines: blue. Progression and CIS status is shown below the sample identifiers (black box: progression and CIS respectively). Yellow represents up-regulation and blue down-regulation compared to the median expression of the miRNA (black).

### DETAILED DESCRIPTION OF THE INVENTION

The invention features compositions and methods that are useful for detection, classification and diagnosis of bladder cancer. The invention relates to the identification of sets of marker miRNAs which are specific for particular tumor classes. The marker miRNAs identified as most significantly indicative of bladder cancer, bladder cancer stage, bladder cancer progression, and/or presence of bladder cancer carcinoma-in-situ (CIS) are shown in Table 3.

In one embodiment, the miRNA expression markers described herein can be used to identify or classify bladder cancer tumors. In this embodiment, a bladder cancer tumor sample is obtained and the miRNA expression pattern of a set of miRNAs identified in Table 3 is determined. For example, the nucleic acid molecules within the sample can be rendered available for hybridization to an oligonucleotide array as described in the Examples. Alternatively, the expression of the miRNAs identified herein can be assessed using any art-recognized quantitative and/or semi-quantitative means of miRNA detection, e.g., quantitative PCR (QPCR), Northern blot, etc. The marker miRNAs to be assessed can be all or a portion of the marker miRNAs associated with a single particular bladder cancer tumor stage, or can be all or a portion of the marker miRNAs associated with several different bladder cancer tumor stages.

The stage of a bladder tumor indicates how deep the tumor has penetrated. Superficial tumors are termed Ta, while T1, T2, T3 and T4 are used to describe increasing degrees of penetration into the muscle. The grade of a bladder tumor is expressed on a scale of I-IV (1-4) according to Bergkvist et al. (1965) Acta Chir Scand, 130: 371-8. The grade reflects the cytological appearance of the cells. Grade I cells are almost normal. Grade II cells are slightly deviant. Grade III cells are clearly abnormal. And Grade IV cells are highly abnormal. A special form of bladder malignancy is carcinoma-in-situ or dysplasia-in-situ in which the altered cells are located in-situ.

It is important to classify the stage of a cancer disease, as superficial tumors may require a less intensive treatment than invasive tumors. According to the invention, the expression level of miRNAs may be used to identify miRNAs whose expression can be used to identify a certain stage of the disease. These “Classifiers” are divided into those which can be used to identify Ta, T1, T2, T3, and T4 stages. In one aspect of the invention, measuring the expression level of one or more of these miRNAs may lead to a classifier that can add supplementary information to the information obtained from the pathological T2 classification.

### DEFINITIONS

The term “cancer,” as used herein, refers to the disease that is typically characterised by abnormal or unregulated cell growth, capable of invading adjacent tissues and spreading to distant organs. The term “carcinoma” refers to the tissue resulting from abnormal or unregulated cell growth. The term “bladder cancer” refers to a cancer found upon, within, or originating from, the bladder. The term “tumor” refers to any abnormal mass of tissue generated by a neoplastic process, whether this is benign (non cancerous) or malignant (cancerous).

The stage of a bladder tumor indicates how deep the tumor has penetrated. Superficial tumors are termed Ta, while T1, T2, T3 and T4 are used to describe increasing degrees of penetration into the muscle. The grade of a bladder tumor is expressed on a scale of I-IV (1-4) according to Bergkvist et al. (1965) Acta Chir Scand. 130: 371-8. The grade reflects the cytological appearance of the cells. Grade I cells are almost normal. Grade II cells are slightly deviant. Grade III cells are clearly abnormal. And Grade IV cells are highly abnormal. A special form of bladder malignancy is carcinoma-in-situ or dysplasia-in-situ in which the altered cells are located in-situ.

It is important to classify the stage of a cancer disease, as superficial tumors may require a less intensive treatment than invasive tumors. According to the invention, the expression level of miRNAs may be used to identify miRNAs whose expression can be used to identify a certain stage of the disease. These “Classifiers” are divided into those which can be used to identify Ta, T1, T2, T3, and T4 stages. In one aspect of the invention, measuring the expression level of one or more of these miRNAs may lead to a classifier that can add supplementary information to the information obtained from, e.g., a pathological Ta or T1 classification.

The term “a Ta or T1 stage bladder cancer that has subsequently progressed,” as used herein, refers to bladder cancer that is Ta or T1 stage bladder cancer at the time miRNA expression level(s) are determined and that has progressed to a subsequent bladder cancer stage (e.g., T2, T3 or T4) following such determination of miRNA expression level(s).

The term “an advanced stage bladder cancer sample,” as used herein, refers to a bladder cancer sample of at least stage T2, and comprises, e.g., stage T2, stage T3 and stage T4 bladder cancers (bladder cancer tumors).

The two essential features of cancer are invasion and metastasis. At one extreme, microinvasion of the basement membrane characterizes the transition from neoplasia to cancer, and at the other extreme, metastases generally lead to death.

Invasion into the underlying connective tissue by a primary tumor proceeds in stages and is facilitated by various mediators produced by the tumor cells. Tumor cells that have not invaded the basement membrane and remain confined within the epithelium are termed carcinoma in situ. The term “carcinoma in situ (CIS),” accordingly, refers to malignant cells that are not yet invasive and have not yet left the site of their origin.

The term “miRNA” or “microRNA” refers to a non coding small RNA produced by a DICR enzyme from a double stranded RNA Precursor. The precursor has a stem loop or hairpin structure. miRNA are present in animals or plants. They can bind to a protein complex termed RISC (or, specifically, miRISC). They represent one of the components/substrates of the RNA inhibitory pathway (RNAi), together with other components/substrates like siRNA.

The terms “RNA molecule”, “miRNA molecule” “mRNA molecule”, “DNA molecule”, “cDNA molecule”, and “nucleic acid molecule” are each intended to cover a single molecule, a plurality of molecules of a single species, and a plurality of molecules of different species. The term “miRNA molecule” is also intended to cover both mature and pre-miRNA molecules. Consistent with microarray terminology, “target miRNA” refers to a miRNA or complementary cDNA sequence to be labeled, while “miRNA probe” refers to an unlabeled sense or antisense miRNA sequence attached directly to a microarray support. The term “capture sequence” refers to any non-native nucleotide sequence capable of binding to a “capture reagent sequence”, while the term “capture reagent” refers to a reagent containing a detectable molecule or molecules and a capture reagent sequence or sequences complementary to the capture sequence.

The miRNAs assessed in certain aspects of the invention are RNA molecules that result from transcription of miRNA regions and that either have been identified or are identified to possess predictive value with respect to a condition (e.g., cancer progression, subject survival, etc.). In certain aspects, the present invention can be used effectively to analyze nucleic acid molecules that are the result of transcription, particularly of the miRNAs identified herein. The nucleic acid molecule levels measured can be derived directly from the miRNA or, alternatively, from a corresponding regulatory sequence element or from a gene that the miRNA regulates. All forms of miRNAs can be measured. For example, a miRNA may be measured in a full-length form, in a cleaved (e.g., Dicer-cleaved) form, or may be detected via detection of only guide (antisense) strand or sense strands derived from and/or corresponding to such a miRNA. Additionally, variants of miRNAs including, for example, polymorphic alleles, can be measured. The sample to be assessed can be any sample that contains a miRNA. Suitable sources of miRNAs, e.g., samples, can include intact cells, lysed cells, cellular material for determining miRNA expression, or material containing miRNAs. Examples of such samples are cells or tissue derived from the bladder or surrounding tissues. Methods of obtaining such samples are known in the art.

As used herein, the term “miRNA expression level” is intended to mean the amount, accumulation or rate of synthesis of a miRNA. The expression level of a miRNA can be represented, for example, by the amount or synthesis rate of a miRNA (or precursor form thereof) encoded by a DNA sequence, or the amount or synthesis rate of a biochemical form (e.g., cleavage product, mature form, single strand of a mature miRNA, etc.) of such a miRNA accumulated in a cell. The meaning of the term “miRNA expression level” can be used to refer to an absolute amount of a miRNA molecule in a sample or to a relative amount of the miRNA molecule, including amounts determined under steady-state or non-steady-state conditions. The miRNA expression level of a molecule can be determined relative to a control component molecule in a sample (e.g., in comparison to the expression level of a “housekeeping gene,” control miRNA, etc.).

The term “modulated” refers to an alteration in the miRNA expression level (induction or repression) to a measurable or detectable (and statistically significant) degree, as compared to a pre-established standard(s) (for example, the expression level of a miRNA in a selected tissue or cell type at a selected phase under selected conditions).

By “miRNA expression pattern” is meant the level or amount of miRNA expression of a plurality of miRNAs, for example, a plurality of miRNAs as assessed by methods described herein or as known in the art. The miRNA expression pattern can comprise data for two or more miRNAs and can be measured at a single time point or over a period of time. For example, the miRNA expression pattern can be determined using two miRNAs, or it can be determined using three or more miRNAs, four or more miRNAs, five or more miRNAs, eight or more miRNAs, twenty or more miRNAs, or fifty or more miRNAs. A miRNA expression pattern may include expression levels of miRNAs that are not specific to a particular tumor or tumor class/stage, as well as genes that are specific to a particular tumor (e.g., bladder cancer tumor) or tumor class/stage (e.g., Ta, T1, T2, T3, T4 stage bladder cancer tumors, CIS, etc.). The miRNAs comprising a miRNA expression pattern can be upregulated, downregulated, or embody no change in comparison of a test miRNA expression pattern with a standard miRNA expression pattern. Classification (e.g., the presence or absence of bladder cancer, the determination of a stage associated with a bladder cancer tumor, the determination of the probability of invasiveness and/or malignancy associated with a bladder cancer tumor, or the identification of a compound that modulates tumor development) can be made by comparing the miRNA expression pattern of the sample with respect to one or more miRNAs with one or more miRNA expression patterns specific to a particular tumor or tumor class (e.g., in a database). Using the methods described herein, expression of numerous miRNAs can be measured simultaneously. The assessment of numerous miRNAs provides for a more accurate evaluation of the sample because there are more miRNAs that can assist in classifying the sample.

The miRNA expression pattern obtained can be compared with the miRNA expression pattern(s) associated with a control tissue and/or with the expression pattern(s) associated with one or more stages and/or classes of bladder cancer tumors as described herein. A classification of the bladder cancer tumor can be made based on the difference between the miRNA expression pattern obtained for a test sample and the miRNA expression pattern associated with a control (e.g., non-cancerous) tissue. Additionally or alternatively, such a classification can be made based on assessment of the similarity or identity of the test sample miRNA expression pattern and the miRNA expression pattern characteristic of a particular bladder cancer tumor stage and/or class. For example, it may be determined that the miRNA expression pattern of the miRNAs tested correlates most closely with the miRNA expression pattern characteristic of invasive bladder cancer tumors (e.g., T2, T3 and/or T4 stage bladder cancer tumors), and a determination can be made that the test sample contains a tumor type that is more likely to progress in an invasive and/or metastatic manner if not completely removed and/or treated. As described further herein, bladder cancer tissues that exhibit miRNA expression patterns resembling aggressive forms of bladder cancer tumors are also likely to be predictive of decreased survival time for a subject and/or the presence and/or development of the condition of carcinoma-in-situ (CIS).

A miRNA expression pattern can be represented in any art-recognized form, e.g., by an expression profile “heat map,” by an array of values corresponding to absolute and/or relative measurements of miRNA expression levels, etc. A selection of the information present in a miRNA expression pattern may also be represented/summarized as a single score or compressed array of scores, e.g., through use of an algorithm that allows for calculation of such a representative/summary score(s) from a miRNA expression pattern (e.g., summarized from an array of individual miRNA expression levels). As is known in the art, such an algorithm may optionally allow for weighting of the relative contribution(s) of individual miRNAs to a calculated score(s). Such representative/summary scores may be compared as proxies in lieu of or in addition to comparing test sample and control sample miRNA expression patterns directly.

A “standard miRNA expression level” of a control tissue, bladder cancer tissue, etc., as used herein, may reflect or contain information derived from not only a single expression level measurement for a miRNA, but may also reflect any standard expression level for the miRNA that may be derived from one or a multiplicity of such individual expression levels for such samples or tissues for the miRNA. For example, in certain embodiments of the invention, a test sample miRNA expression level may be correlated with and/or compared to a standard miRNA expression level that is representative of a plurality of different miRNA expression level measurements for the miRNA performed upon multiple individual samples to arrive at, e.g., a mean, median or other (e.g., statistically manipulated or otherwise summarized or aggregated) representative miRNA expression level for the miRNA across a number of or all such control samples. Such mean, median, summarized or aggregated standard expression levels that reflect information derived from, e.g., multiple individual underlying expression levels for a miRNA, are considered a “standard miRNA expression level” for purpose of the instant invention.

A “standard expression pattern” of a control tissue, bladder cancer tissue, etc., as used herein, may reflect or contain information derived from not only a single expression pattern obtained for a single tissue sample, but may also reflect any standard expression pattern that may be derived from one or a multiplicity of such individual expression patterns for such samples or tissues. For example, in certain embodiments of the invention, a test sample expression pattern may be correlated with and/or compared to a standard expression pattern that incorporates information from a plurality of different miRNA expression level measurements performed upon multiple individual samples to arrive at, e.g., mean, median or other (e.g., statistically manipulated or otherwise summarized or aggregated) expression levels for a plurality of miRNAs across a number of or all such control samples. Such aggregated, summarized or “signature” standard expression patterns that reflect information derived from, e.g., multiple individual underlying expression patterns, are considered “standard expression patterns” for purpose of the instant invention. As described above, such “signature” expression patterns, as is true for any “standard expression pattern,” may further be summarized as a single score or compressed array of scores, e.g., via implementation of a weighted algorithm, as is known in the art and referenced above.

As used herein, “a plurality of” or “a set of” refers to more than one, for example, 2 or more, 3 or more, 4 or more, 5 or more, 6 or more, 7 or more, 8 or more, 9 or more, 10 or more, 15 or more, 20 or more, 25 or more, 30 or more, 35 or more, 40 or more, 45 or more, 50 or more, etc.

The term “correlation” unless indicated otherwise, is used herein to indicate that a “statistical association” exists between, e.g., a sample miRNA expression profile and a standard miRNA expression profile or standard miRNA expression signature. Computerized assessment of the extent of statistical association between expression profiles (e.g., mRNA expression profiling software that can also be applied to miRNA expression profiling) is well known in the art. Exemplary means of assessing the extent of correlation between miRNA expression profiles includes but is not limited to SAM (Significance Analysis of Microarrays) implemented in TIGR MEV 3.1 software. Many other means of analyzing expression patterns or signatures are known in the art and can be employed with the present invention.

The terms “diagnostic assay” and “diagnostic method” refer to the detection of the presence or nature of a pathologic condition. Diagnostic assays differ in their sensitivity and specificity. For example, for embodiments related to diagnosis of bladder cancer, subjects who test positive for bladder cancer and are actually diseased are considered “true positives”. Within the context of the invention, the sensitivity of a diagnostic assay is defined as the percentage of the true positives in the diseased population. Subjects having bladder cancer but not detected by the diagnostic assay are considered “false negatives”. Subjects who are not diseased and who test negative in the diagnostic assay are considered “true negatives”. The term specificity of a diagnostic assay, as used herein, is defined as the percentage of the true negatives in the non-diseased population. For the various classifier embodiments of the invention, specificity and sensitivity considerations are similarly applied.

In certain embodiments, the present invention describes and/or employs arrays (e.g., microarrays) having multiple single oligonucleotide sequences arranged in specific locations thereon and being identical or complementary to miRNA present in the cells, tissues, extracts, etc., for which the presence of and/or expressed levels of miRNA are to be determined.

The methods of the invention embrace the establishment of databases that contain miRNA expression level or miRNA expression pattern data for a variety of normal and abnormal bladder and bladder cancer tissues, and comparison of miRNA expression data from test tissue samples to such databases. Accordingly, the miRNA expression level or miRNA expression pattern from a test tissue sample can be compared to a standard control from the same or a different subject prepared concurrently with (or prior to) the test tissue sample, or to miRNA expression level(s) or miRNA expression patterns previously determined for one or more abnormal (and optionally normal) tissue types.

In certain embodiments, the invention provides for measurement of miRNAs that are identified as elevated in association with bladder cancer. Such miRNAs include miR-193a, miR-519e*, miR-21, miR-202 and miR-198, and certain classifiers of the invention that employ these miRNAs identify a miRNA selected from this group of miRNAs to be elevated if the measured expression level value for the miRNA is more than 1.2-fold higher, optionally more than 1.3-fold higher, more than 1.4-fold higher, more than 1.5-fold higher, more than 2-fold higher, more than 3-fold higher or more than 4-fold higher in a test sample tissue relative to the measured (and/or mean, median, etc.) level of the miRNA in a control tissue and/or within a standard expression pattern. It is noted that for miRNA expression measurement, the raw fold change values obtained from assessment of miRNA array data and presented, e.g., in Table 3, are represented as log₂ values. Thus, a raw fold change value of 1.9 for hsa-miR-21 in normal versus tumor comparisons identifies a 3.7-fold (2¹⁹-fold) elevation of hsa-miR-21 in tumor tissue relative to normal (control) tissue.

In certain embodiments, the invention provides for measurement of miRNAs that are identified as reduced in association with bladder cancer. Such miRNAs include miR-145, miR-455, miR-143, miR-125b and miR-126, and certain classifiers of the invention that employ these miRNAs identify a miRNA selected from this group of miRNAs to be reduced if the measured expression level value for the miRNA is more than 1.2-fold lower, optionally more than 1.3-fold lower, more than 1.4-fold lower, more than 1.5-fold lower, more than 2-fold lower, more than 3-fold lower or more than 4-fold lower in a test sample tissue relative to the measured (and/or mean, median, etc.) level of the miRNA in a control tissue and/or within a standard expression pattern.

In certain embodiments, the invention provides for measurement of miRNAs that are identified as elevated in association with progressing bladder cancer. Such miRNAs include miR-129, miR-133b, miR-320, miR-503, miR-373*, miR-526c, miR-498, miR-518c*, miR-185, miR-483 and miR-145, and certain classifiers of the invention that employ these miRNAs identify a miRNA selected from this group of miRNAs to be elevated if the measured expression level value for the miRNA is more than 1.2-fold higher, optionally more than 1.3-fold higher, more than 1.4-fold higher, more than 1.5-fold higher, more than 2-fold higher, more than 3-fold higher or more than 4-fold higher in a test sample tissue relative to the measured (and/or mean, median, etc.) level of the miRNA in a control tissue and/or within a standard expression pattern.

In certain embodiments, the invention provides for measurement of miRNAs that are identified as reduced in association with progressing bladder cancer. Such miRNAs include miR-29c, miR-29b, miR-29a and miR-203, and certain classifiers of the invention that employ these miRNAs identify a miRNA selected from this group of miRNAs to be reduced if the measured expression level value for the miRNA is more than 1.2-fold lower, optionally more than 1.3-fold lower, more than 1.4-fold lower, more than 1.5-fold lower, more than 2-fold lower, more than 3-fold lower or more than 4-fold lower in a test sample tissue relative to the measured (and/or mean, median, etc.) level of the miRNA in a control tissue and/or within a standard expression pattern.

In certain embodiments, the invention provides for measurement of miRNAs that are identified as elevated in association with advanced stage bladder cancer. Such miRNAs include miR-129, miR-503, miR-320, miR-498 and miR-483, and certain classifiers of the invention that employ these miRNAs identify a miRNA selected from this group of miRNAs to be elevated if the measured expression level value for the miRNA is more than 1.2-fold higher, optionally more than 1.3-fold higher, more than 1.4-fold higher, more than 1.5-fold higher, more than 2-fold higher, more than 3-fold higher or more than 4-fold higher in a test sample tissue relative to the measured (and/or mean, median, etc.) level of the miRNA in a control tissue and/or within a standard expression pattern.

In certain embodiments, the invention provides for measurement of miRNAs that are identified as reduced in association with advanced stage bladder cancer. Such miRNAs include let-7g, miR-18b, miR-200b, miR-141 and miR-219, and certain classifiers of the invention that employ these miRNAs identify a miRNA selected from this group of miRNAs to be reduced if the measured expression level value for the miRNA is more than 1.2-fold lower, optionally more than 1.3-fold lower, more than 1.4-fold lower, more than 1.5-fold lower, more than 2-fold lower, more than 3-fold lower or more than 4-fold lower in a test sample tissue relative to the measured (and/or mean, median, etc.) level of the miRNA in a control tissue and/or within a standard expression pattern.

In certain embodiments, the invention provides for measurement of miRNAs that are identified as elevated in association with carcinoma-in-situ. Such miRNAs include miR-129, miR-503, miR-498, miR-483, miR-451, miR-373*, miR-451 and miR-320, and certain classifiers of the invention that employ these miRNAs identify a miRNA selected from this group of miRNAs to be elevated if the measured expression level value for the miRNA is more than 1.2-fold higher, optionally more than 1.3-fold higher, more than 1.4-fold higher, more than 1.5-fold higher, more than 2-fold higher, more than 3-fold higher or more than 4-fold higher in a test sample tissue relative to the measured (and/or mean, median, etc.) level of the miRNA in a control tissue and/or within a standard expression pattern.

In certain embodiments, the invention provides for measurement of miRNAs that are identified as reduced in association with carcinoma-in-situ. Such miRNAs include miR-205, miR-382, miR-200a*, miR-487a, miR-191*, miR-29c, miR-210, miR-302b, miR-324-5p, miR-127, miR-31, miR-345 and miR-202*, and certain classifiers of the invention that employ these miRNAs identify a miRNA selected from this group of miRNAs to be reduced if the measured expression level value for the miRNA is more than 1.2-fold lower, optionally more than 1.3-fold lower, more than 1.4-fold lower, more than 1.5-fold lower, more than 2-fold lower, more than 3-fold lower or more than 4-fold lower in a test sample tissue relative to the measured (and/or mean, median, etc.) level of the miRNA in a control tissue and/or within a standard expression pattern.

In certain embodiments, the invention provides for measurement of the expression level of miR-129 as elevated in association with bladder cancer that poses a significant survival risk to a subject. For such measurements, miR-129 is identified as elevated in a subject if the measured expression level value for miR-129 is more than 1.2-fold higher, optionally more than 1.3-fold higher, more than 1.4-fold higher, more than 1.5-fold higher, more than 2-fold higher, more than 3-fold higher or more than 4-fold higher in a test sample tissue relative to the measured (and/or mean, median, etc.) level of the miR-129 in a control tissue and/or within a standard expression pattern.

In certain embodiments, elevation of miR-129 at least 1.3-fold in a subject relative to an appropriate control indicates the presence of advanced stage (T2 to T4) bladder cancer in the subject. In related embodiments, elevation of miR-129 at least 1.3-fold in a subject relative to an appropriate control indicates the presence of a progressing bladder cancer in the subject. In additional embodiments, elevation of miR-129 at least 1.3-fold in a subject indicates a poor prognosis for bladder cancer in the subject. In further embodiments, elevation of miR-129 at least 1.3-fold in a subject indicates the presence of carcinoma-in-situ (CIS). Measurement of such elevated levels of miR-129 can also identify a subject in need of cancer therapy.

In certain embodiments, the invention provides for measurement of the expression level of miR-29c as reduced in association with bladder cancer that poses a significant survival risk to a subject. For such measurements, miR-29c is identified as reduced in a subject if the measured expression level value for miR-29c is more than 1.2-fold lower, optionally more than 1.3-fold lower, more than 1.4-fold lower, more than 1.5-fold lower, more than 2-fold lower, more than 3-fold lower or more than 4-fold lower in a test sample tissue relative to the measured (and/or mean, median, etc.) level of the miR-29c in a control tissue and/or within a standard expression pattern.

In certain embodiments, reduction of miR-29c at least 1.2-fold in a subject relative to an appropriate control indicates the presence of progressing bladder cancer in the subject. In related embodiments, reduction of miR-29c at least 1.2-fold in a subject relative to an appropriate control indicates the presence of carcinoma-in-situ (CIS). Measurement of such elevated levels of miR-29c can also identify a subject in need of cancer therapy.

The term “chemotherapy” generally refers to a treatment of a disease using specific chemical agents. In the present invention, a subject of chemotherapy can be a cancer cell or tissue, preferably a bladder cancer cell or tissue (e.g., tumor). Herein, “chemotherapeutic agent” refers to a pharmaceutical agent generally used for treating cancer, particularly bladder cancer. The chemotherapeutic agents for treating cancer include, for example, cisplatin, carboplatin, etoposide, vincristine, cyclophosphamide, doxorubicin, ifosfamide, paclitaxel, gemcitabine, and docetaxel.

In addition to the general cancer chemotherapy treatments described above, bladder cancer may also be treated with art-recognized treatments that have shown particular efficacy against bladder cancer, such as BCG and mitomycin C. BCG is an attenuated strain of Mycobacterium bovis that has been used as a vaccine against tuberculosis. It was first used to treat bladder cancer in 1976. BCG is a potent stimulator of host defense mechanisms and can induce tumour regression in immunocompetent hosts. Repeated instillation of BCG produces a nonspecific immune reaction within the bladder which allows the cells of the lining to be shed, especially those rapidly dividing cancer cells. BCG is usually given after superficial bladder tumors have been removed during an operation. BCG is administered intravesically via a catheter tube that is placed into the bladder and is administered repeatedly over a period of time. BCG has emerged as an effective intravesical treatment for superficial bladder cancer and carcinoma in situ (CIS) (Alexandroff et al., Lancet, 1999, 353:1689). The effects of BCG in bladder cancer are local only, and there is no protection against the development of tumors in areas where there is no BCG contact.

Mitomycin C is an antitumor antibiotic isolated from Streptomyces caespitosus. Clinical trials with Mitomycin C began in the U.S. in the late 1960's. In viva, mitomycin C was activated to a bifunctional and trifunctional alkylating agent which binds to DNA. Its binding to DNA leads to cross-linking and inhibition of DNA synthesis and function. When administered intravesically in the treatment of bladder cancer, mitomycin C is equivalent to BCG in the treatment of bladder cancer, particularly with respect to survival rate.

The term “cancer therapy,” as used herein, refers to any art-recognized treatment used to treat a cancer/tumor that has been identified as likely to progress, metastasize, or generally threaten the health or well-being of a subject. Examples of “cancer therapy” for bladder cancers include surgical resection of cancer and/or tumor cells, optionally including resection of surrounding tissue, chemotherapy, BCG and/or mitomycin C administration as described above, regular medical evaluation/re-evaluation of cancer status (e.g., following chemotherapy, surgery, etc.), or other art-recognized approach to treating a subject with/ridding a subject of a cancer (e.g., bladder cancer tumor) that presents a threat to the health or well-being of a subject.

The term “quantitative PCR”, or “Q-PCR” refers to a variety of well known methods used to quantify the results of the polymerase chain reaction for specific nucleic acid sequences. Such methods typically are categorized as kinetics-based systems, that generally determine or compare the amplification factor, such as determining the threshold cycle (Ct), or as co-amplification methods, that generally compare the amount of product generated from simultaneous amplification of target and standard templates. Many Q-PCR techniques comprise reporter probes, intercalating dyes, or both. For example, but not limited to, TaqMan® probes (Applied Biosystems), i-probes, molecular beacons, Eclipse probes, scorpion primers, Lux™ primers, FRET primers, ethidium bromide, SYBR® Green I (Molecular Probes), and PicoGreen® (Molecular Probes).

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a selected: embodiment, hybridization will occur at 30° C. C. in 750 mM NaCl, 75 mM trisodium citrate, and 1% SDS. In a further selected embodiment, hybridization will occur at 37° C. C. in 500 mM NaCl, 50 mM trisodium citrate, 1% SDS, 35% formamide, and 100 mu.g/ml denatured salmon sperm DNA (ssDNA). In an additional selected embodiment, hybridization will occur at 42° C. C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a selected embodiment, wash steps will occur at 25° C. in 30 mM NaCl, 3 mM trisodium citrate, and 0.1% SDS. In a further selected embodiment, wash steps will occur at 42 degree. C. in 15 mM NaCl, 1.5 mM trisodium citrate, and 0.1% SDS. In an additional selected embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

The present invention provides methods of treating disease and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a cancer, e.g., bladder cancer, disease or disorder or symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g., opinion) or objective (e.g., measurable by a test or diagnostic method).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which cancer, e.g., bladder cancer, may be implicated.

miRNAs Useful in the Diagnosis, Classification and Prognosis of Bladder Cancer

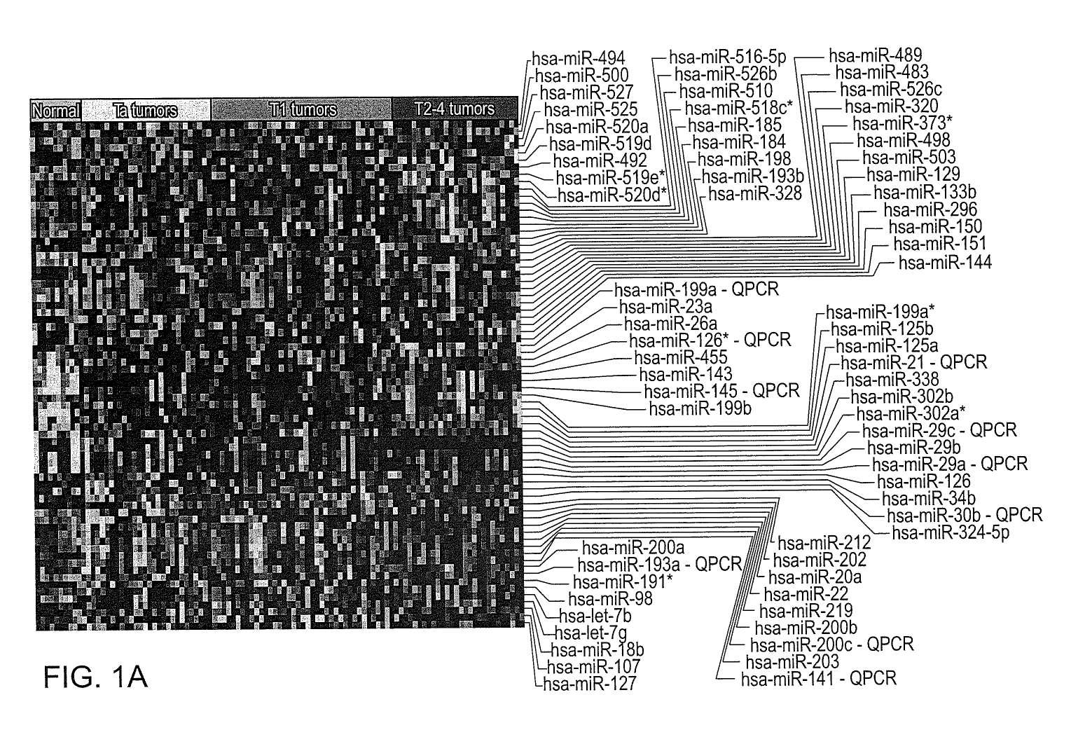

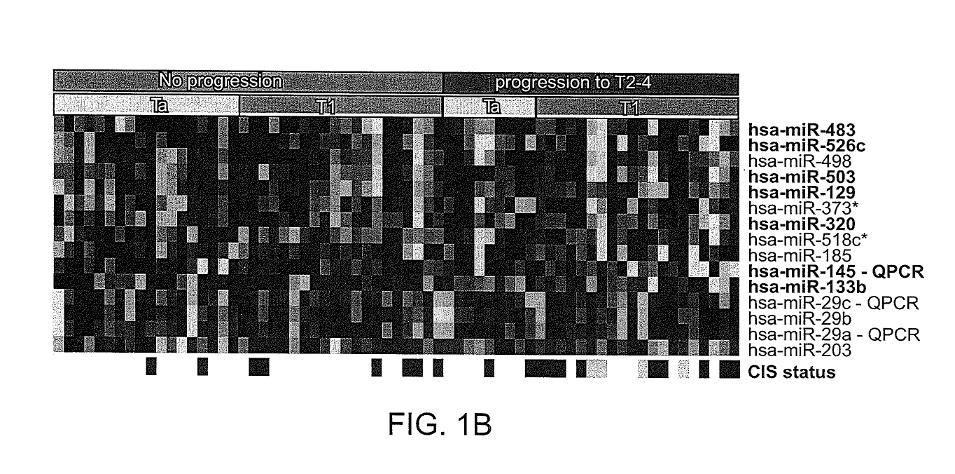

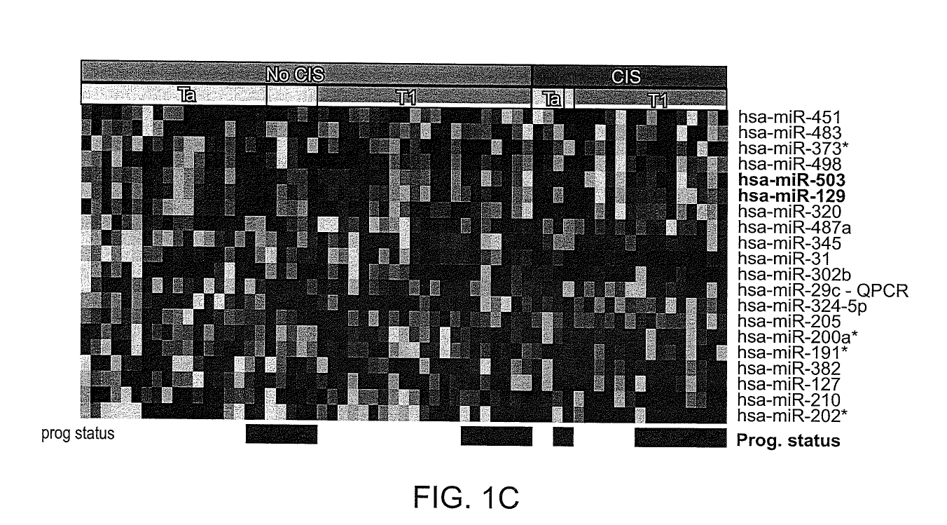

The invention provides sets of miRNAs whose expression is correlated with the diagnosis, prognosis and/or classification of bladder cancer. These miRNAs are listed in Table 3 and FIGS. 1A-1C herein. These miRNAs are particularly useful in the classification of bladder cancer stage (e.g., Ta, T1, T2, T3, T4), progression/non-progression propensity and/or clinical status of bladder cancer, presence of carcinoma-in-situ (CIS) in bladder cancer, and for prognosis of survival and appropriate treatment regimen in a subject with bladder cancer.

In one embodiment, the invention provides a set of 15 bladder cancer progression-informative miRNAs, i.e., miRNAs that are significantly correlated with progression from early stages of bladder cancer (e.g., Ta or T1 stages) to later stages of bladder cancer (invasive bladder cancer stages T2, T3 and T4). These miRNAs are listed in FIG. 1B and in Table 3.

In another embodiment, the invention provides a set of 10 miRNAs possessing informative value for classifying the stage of bladder cancer, i.e., miRNAs that are significantly correlated with non-invasive/pre-invasive Ta or T1 stages of bladder cancer or with invasive stages of bladder cancer (e.g., T2, T3 or T4 stage bladder cancers). These miRNAs are listed in Table 3 and in FIGS. 1A-1C.

In a further embodiment, the invention provides a set of 10 miRNAs possessing informative value for identification of bladder cancer, i.e., miRNAs that are significantly correlated with bladder cancer relative to normal tissue (e.g., normal urothelium tissue). These miRNAs are listed in Table 3 and in FIGS. 1A-1C.

In a further embodiment, the invention provides a set of 20 miRNAs possessing informative value for identification of carcinoma-in-situ, i.e., miRNAs that are significantly correlated with carcinoma-in-situ in bladder cancer relative to tissue and/or bladder cancer that is not carcinoma-in-situ. These miRNAs are listed in Table 3 and in FIGS. 1A-1C.

The invention also provides subsets of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35 at least 40, at least 45 or at least 50 of the different miRNAs present in Table 3 or in FIGS. 1A-1C, which are useful for diagnosis, classification and/or prognosis of bladder cancer in individuals having or at risk of developing bladder cancer. The invention further provides subsets of no more than 2, 3, 4, 5, 6, 7, 8, 9, 10, 11, 12, 13, 14, 15, 20, 25, 30, 35, 40, 45, 50 or 55 of the different miRNAs in Table 3 or in FIGS. 1A-1C, which are useful for diagnosis, classification and/or prognosis of bladder cancer in individuals having or at risk of developing bladder cancer. The invention further provides subsets of at least 10%, 15%, 20%, 25%, 30%, 35%, 40%, 45%, 50%, 60%, 70%, 80%, or 90% of the different miRNAs listed in Table 3 or in FIGS. 1A-1C.

Identification of Informative miRNAs

The present invention provides sets of miRNAs for the detection, identification, classification and/or prognosis of bladder cancer or conditions or indications associated with bladder cancer. Generally, the miRNA sets were identified by determining which of 315 human miRNAs had expression patterns that correlate with bladder cancer or the conditions or indications of bladder cancer.

The methods for identification of sets of informative miRNAs make use of Measured cellular constituent profiles, e.g., expression profiles of a plurality of miRNAs, in tumor samples from a plurality of patients. In certain embodiments of the invention, especially those related to predicting disease progression and/or responsiveness to bladder cancer treatment, patient prognosis outcomes are known. The prognosis outcomes can be the prognosis at a predetermined time after initial diagnosis. The predetermined time can be any appropriate time period, e.g., 1, 2, 3, 4, or 5 years. Prognosis miRNAs can be obtained by identifying miRNAs whose expression levels correlate with prognosis outcome, e.g., miRNAs whose expression levels in good prognosis patients are significantly different from those in poor prognosis patients. In selected embodiments, the tumor samples from the plurality of patients are separated into a good prognosis group and a poor prognosis group for the predetermined time period. miRNAs whose expression levels exhibit differences between the good and poor prognosis groups to at least a predetermined level are selected as the miRNAs whose expression levels correlate with patient prognosis. This section describes embodiments which employ miRNAs as informative for bladder cancer detection, classification, diagnosis and/or prognosis. However, it will be understood by a person skilled in the art that gene expression levels and/or profiles, proteins or other cellular constituents (e.g., genotype status, e.g., microsatellite, SNP, etc.) may also be incorporated into a bladder cancer classifier and/or predictor that employs miRNA expression level and/or expression pattern measurements as described herein.

In a selected embodiment, the miRNA expression profile is a differential miRNA expression profile. Each measurement in the profile is a differential expression level of a miRNA in a bladder cancer sample versus that in a standard sample (also termed a reference or control sample). In one embodiment, the standard sample comprises polynucleotide molecules, derived from one or more samples from a plurality of normal individuals. For example, the normal individuals may be persons not having bladder cancer. The standard or control may also comprise polynucleotide molecules, derived from one or more samples derived from individuals having a different form or stage of bladder cancer; a different disease or different condition, or individuals exposed or subjected to a different condition, than the individual from which the sample of interest was obtained. The standard sample may also be a “positive control” standard sample, e.g., comprising polynucleotide molecules derived from one or more samples derived from individuals having a form and/or stage of bladder cancer that is used in comparison to the expression pattern of the sample of interest and may be determined to be the same, similar or divergent from that of the sample of interest. The standard or reference or control may also be a sample, or set of samples, taken from the individual at an earlier time, for example, to assess the progression of a condition, or the response to a course of therapy.

In one embodiment, the standard or control is a pool of target polynucleotide molecules derived from a plurality of different individuals. In a selected embodiment in the context of bladder cancer, the pool comprises samples taken from a number of individuals having Ta and/or T1 bladder cancer tumors.

In another embodiment, the pool comprises an artificially-generated population of nucleic acids designed to approximate the level of nucleic acid derived from each miRNA found in a pool of miRNAs derived from tumor samples. In another embodiment, the pool, also called a “mathematical sample pool,” is represented by a set of expression values, rather than a set of physical polynucleotides; the level of expression of relevant miRNAs in a sample from an individual with a condition, such as a disease, is compared to values representing control levels of expression for the same miRNAs in the mathematical sample pool. Such a control may be a set of values stored on a computer. Such artificial or mathematical controls may be constructed for any condition of interest.

In another embodiment, the reference sample is derived from a normal bladder cell line or a bladder cancer cell line. Of course, where, for example, expressed miRNAs are used, the miRNAs are obtained from the individual's sample, and the standard or control could be a pool of miRNAs from a number of normal individuals, or from a number of individuals having a particular state of a condition, such as a pool of samples from individuals having a particular prognosis of bladder cancer.

In one embodiment, the method for identifying miRNA sets is as follows.

After extraction and labeling of target polynucleotides, the expression of all miRNAs assessed in a sample X is compared to the expression of all miRNAs assessed in a standard or control. In one embodiment, the standard or control comprises target polynucleotide molecules derived from a sample from a normal individual (i.e., an individual not having bladder cancer). In a selected embodiment, the standard or control is a pool of target polynucleotide molecules.

The pool may be derived from collected samples from a number of normal individuals. In one embodiment, the pool comprises samples taken from a number of individuals having Ta or T1 stage bladder cancer tumors. In another embodiment, the pool comprises an artificially-generated population of nucleic acids designed to approximate the level of nucleic acid derived from each miRNA found in a pool of miRNAs derived from tumor samples. In yet another embodiment, the pool is derived from normal or bladder cancer cell lines or cell line samples.

The comparison may be accomplished by any means known in the art. For example, expression levels of various miRNAs may be assessed by separation of target polynucleotide molecules (e.g., RNA or cDNA) derived from the miRNAs in agarose or polyacrylamide gels, followed by hybridization with miRNA-specific oligonucleotide probes. Alternatively, the comparison may be accomplished by the labeling of target polynucleotide molecules followed by separation on a sequencing gel. Polynucleotide samples are placed on the gel such that patient and control or standard polynucleotides are in adjacent lanes. Comparison of expression levels is accomplished visually or by means of densitometer. In a selected embodiment, the expression of all miRNAs is assessed simultaneously by hybridization to a microarray. In each approach, miRNAs meeting certain criteria are identified as associated with bladder cancer.

In one embodiment, a set of bladder cancer tumor samples are first screened for significant variation in miRNA expression as compared to a standard or control sample. miRNAs may be screened, for example, by determining whether they show significant variation as compared to a standard or control sample in at least some samples among the set of samples. miRNAs that do not show significant variation in at least some samples in the set of samples are presumed not to be informative, and are discarded from further consideration. miRNAs showing significant variation in at least some samples in the sample set are retained as candidate informative miRNAs. The degree of variation in expression of a miRNA may be estimated by determining a difference or ratio of the expression of the miRNA in a sample and a control. The difference or ratio of expression may be further transformed, e.g., by a linear or log transformation. Selection of candidate miRNAs may be made based upon either significant up- or down-regulation of the miRNA in at least some samples in the set or based on the statistical significance (e.g., the p-value) of the variation in expression of the miRNA. Preferably, both selection criteria are used. Thus, in one embodiment of the present invention, miRNAs showing both a more than 1.3-fold change (increase or decrease) in expression as compared to a standard in at least three samples, and a p-value of variation in expression of the miRNA in the set of tumor samples as compared to the standard sample is no more than 0.01 (i.e., is statistically significant) are selected as candidate miRNAs associated with detection, classification and/or prognosis of bladder cancer.

Expression patterns comprising a plurality of different miRNAs in a plurality of n bladder cancer tumor samples can be used to identify miRNAs that correlate with, and therefore are useful for discriminating, different clinical categories. In a specific embodiment using n tumor samples, informative miRNAs are identified by calculation of correlation coefficients between the clinical category or clinical parameter(s) and the linear, logarithmic or any transform of the expression ratio across all samples for each individual miRNA.

Specifically, the correlation coefficient may be calculated as: p=(c·r)(∥c∥∥r∥) Equation (1) where c represents the clinical parameters in the n tumor samples or categories and r represents the measured expression levels of a miRNA in the n tumor samples, e.g., each element in r can be the linear, logarithmic or any transform of the ratio of expression of the miRNA between a tumor sample and a control. miRNAs for which the coefficient of correlation exceeds a cutoff or threshold value are identified as bladder cancer-related miRNAs specific for a particular clinical type, cancer stage, etc. Such a cutoff or threshold value may correspond to a certain significance of discriminating miRNAs obtained by Monte Carlo simulations. The threshold depends upon the number of samples used, and can be calculated as described, e.g., in WO 06/015312, incorporated herein by reference. In a specific embodiment, miRNAs are chosen if the correlation coefficient is greater than about 0.3 or less than about −0.3.

Next, the significance of the set of miRNAs can be evaluated. The significance may be calculated by any appropriate statistical method. In a specific example, a Monte-Carlo technique is used to randomize the association between the expression profiles of the plurality of patients and the bladder cancer stage and/or clinical categories to generate a set of randomized data. The same miRNA selection procedure as used to select the miRNA set is applied to the randomized data to obtain a control miRNA set. A plurality of such runs can be performed to generate a probability distribution of the number of miRNAs in control miRNA sets. In one embodiment, 10,000 such runs are performed. From the probability distribution, the probability of finding a miRNA set consisting of a given number of miRNAs when no correlation between the expression levels and phenotype is expected (i.e., based on randomized data) can be determined. The significance of the miRNA set obtained from the real data can be evaluated based on the number of miRNAs in the miRNA set by comparing to the probability of obtaining a control miRNA set consisting of the same number of miRNAs using the randomized data. In one embodiment, if the probability of obtaining a control miRNA set consisting of the same number of miRNAs using the randomized data is below a given probability threshold, the miRNA set is said to be significant.

Once a miRNA set is identified, the miRNAs may be rank-ordered in order of significance of discrimination. One means of rank ordering is by the amplitude of correlation between the change in expression of the miRNA and the specific condition being discriminated. Another, preferred, means is to use a statistical metric. In a specific embodiment, the metric is a Fisher-like statistic as known in the art.

The rank-ordered miRNA set may be used to optimize the number of miRNAs in the set used for discrimination. This is accomplished generally in a “leave one out” method as follows. In a first run, a subset, for example 5, of the miRNAs from the top of the ranked list is used to generate a template, where out of X samples, X−1 are used to generate the template, and the status of the remaining sample is predicted. This process is repeated for every sample until every one of the X samples is predicted once. In a second run, additional miRNAs, for example 5, are added, so that a template is now generated from 10 miRNAs, and the outcome of the remaining sample is predicted. This process is repeated until the entire set of miRNAs is used to generate the template. For each of the runs, type 1 error (false negative) and type 2 errors (false positive) are counted; the optimal number of miRNAs is that number where the type 1 error rate, or type 2 error rate, or preferably the total of type 1 and type 2 error rate is lowest.

For prognostic miRNAs, validation of the miRNA set may be accomplished by an additional statistic, a survival model. This statistic generates the probability of tumor distant metastasis as a function of time since initial diagnosis. A number of models may be used, including Weibull, normal, log-normal, log logistic, log-exponential, or log-Rayleigh (Chapter 12 “Life Testing”, S-PLUS 2000 GUIDE TO STATISTICS, Vol. 2, p. 368 (2000)).

For the “normal” model, the probability of distant metastasis P at time t is calculated as P=arx exp(−t2/2), where a is fixed and equal to 1, and is a parameter to be fitted and measures the “expected lifetime”.

It will be apparent to those skilled in the art that the above methods, in particular the statistical methods, described above, are not limited to the identification of miRNAs associated with bladder cancer, but may be used to identify set of miRNAs associated with any phenotype. The phenotype can be the presence or absence of a disease such as cancer, or the presence or absence of any identifying clinical condition associated with that cancer. The phenotype may also be the response, or lack thereof, to a particular treatment regimen, for example, a course of one or more anticancer drugs. In the disease context, the phenotype may be a prognosis such as a survival time, probability of distant metastasis of a disease condition, or likelihood of a particular response to a therapeutic or prophylactic regimen. The phenotype need not be cancer, or a disease; the phenotype may be a nominal characteristic associated with a healthy individual.

In another embodiment, the invention provides an “iterative” method for the identification of sets of miRNAs associated with a particular phenotype. An important aspect of this method is that samples, within a set of samples used to construct a classifier for the phenotype (e.g., bladder cancer stage, clinical indication, chemotherapy responsiveness, etc.), that are incorrectly predicted using classifier templates constructed using all samples in the set, are discarded, and samples the phenotype of which is accurately predicted are retained. The retained samples are then used to construct a second classifier, which is more likely to contain a set of miRNAs that reflects the dominant underlying molecular mechanism for the particular phenotype. Such selections of miRNAs can optionally be combined in similar iterative fashion with mRNA expression profiling (identification of informative mRNAs) and/or genotyping, as well as other any other measurements with known informative value in diagnosis, detection, classification and/or prognosis of cancer.

In one embodiment, therefore, the invention provides a method for determining a set of miRNAs whose expression is associated with a particular phenotype, comprising the steps of (a) selecting phenotype having two or more phenotype categories; (b) identifying a first plurality of miRNAs, wherein the expression of said miRNAs in a first plurality of samples is correlated or anticorrelated with one of the phenotype categories; (c) predicting the phenotype category of each sample in said plurality of samples based on the expression level of each of said plurality of miRNAs across all other samples in said plurality of samples; (d) selecting those samples for which the phenotype category is correctly predicted, to form a second plurality of samples; and (e) identifying a second plurality of miRNAs, wherein the expression of said miRNAs in said second plurality of samples is correlated or anticorrelated with one of the phenotype categories; wherein said second plurality of miRNAs is a set of miRNAs whose expression is associated with a particular phenotype. In a specific embodiment, the phenotype is bladder cancer. In a more specific embodiment, said phenotype categories are progression and non-progression. In an even more specific embodiment, said non-progression means no formation of invasive bladder cancer tumors and/or no reoccurrence or metastasis within five years of initial diagnosis of bladder cancer, and progression means formation of invasive bladder cancer tumors and/or reoccurrence or metastasis within five years of initial diagnosis of bladder cancer. In another specific embodiment, said phenotype categories are presence and absence of carcinoma-in-situ in a sample tissue. In a further embodiment, said phenotype may be survival or death in a subject with bladder cancer in a fixed period of time, or response and non-response to a particular anticancer drug, or to a particular combination of anticancer drugs.

This iterative method, of course, may be applied to any disease or condition for which two or more phenotype categories exist. The method may be applied to the original generation of sets of miRNAs informative for a particular phenotype and phenotype category(ies), and may be used to improve existing sets of miRNAs that were selected by less robust means.

It should be noted that each of the miRNAs identified as being phenotype and/or phenotype category-informative may be considered likely targets for therapeutics for that phenotype. For example, miRNAs identified as bladder cancer prognosis-informative represent miRNAs that are targets for therapeutics against bladder cancer (e.g., sequence-specific agents capable of blocking RNAi, specifically of blocking miRNA activity).

### Sample Collection

In the present invention, target polynucleotide molecules are extracted from a sample taken from an individual having bladder cancer. The sample may be collected in any clinically acceptable manner, but must be collected such that miRNA polynucleotides are preserved. miRNA or nucleic acids derived therefrom (i.e., cDNA or amplified DNA) are preferably labeled distinguishably from standard or control polynucleotide molecules, and both are simultaneously or independently hybridized to a microarray comprising some or all of the miRNAs or miRNA sets or subsets described above.

Alternatively, miRNA or nucleic acids derived therefrom may be labeled with the same label as the standard or control polynucleotide molecules, wherein the intensity of hybridization of each at a particular probe is compared. A sample may comprise any clinically relevant tissue sample, such as a tumor biopsy or fine needle aspirate, or a sample of bodily fluid, such as blood, plasma, serum, lymph, ascitic fluid, cystic fluid, urine or nipple exudate. The sample may be taken from a human, or, in a veterinary context, from non-human animals such as ruminants, horses, swine or sheep, or from domestic companion animals such as felines and canines. The sample may also be paraffin-embedded tissue sections (see, e.g., U.S. patent application Publication No. 2005/0048542A1, which is incorporated by reference herein in its entirety). The expression profiles of paraffin-embedded tissue samples are preferably obtained using quantitative polymerase chain reaction QPCR (see section on QPCR infra).

Methods for preparing total and poly(A)⁺ RNA are well known and are described generally in Sambrook et al., MOLECULAR CLONING—A LABORATORY MANUAL (2ND ED.), Vols. 1-3, Cold Spring Harbor Laboratory, Cold Spring Harbor, N.Y. (1989)) and Ausubel et al., CURRENT PROTOCOLS IN MOLECULAR BIOLOGY, vol. 2, Current Protocols Publishing, New York (1994)). Methods for preparation of miRNA, as well as for preparation of high-throughput microarrays developed to identify expression patterns for miRNAs, are known in the art, and include those described, e.g., in Babak et al., RNA 10:1813 (2004); Calm et al., Proc. Natl. Acad. Sci. USA 101:11755 (2004); Liu et al., Proc. Natl. Acad. Sci. USA 101:9740 (2004); Miska et al., Genome Biol. 5:R68 (2004); Sioud and Røsok, BioTechniques 37:574 (2004); Krichevsky et al., RNA 9:1274 (2003); and WO 06/050433, incorporated herein by reference in their entirety.

RNA may be isolated from eukaryotic cells by procedures that involve lysis of the cells and denaturation of the proteins contained therein. Cells of interest include wild-type cells (i.e., non-cancerous), drug-exposed wild-type cells, tumor- or tumor-derived cells, modified cells, normal or tumor cell line cells, and drug-exposed modified cells.

Additional steps may be employed to remove DNA. Cell lysis may be accomplished with a nonionic detergent, followed by microcentrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et al., Biochemistry 18:5294-5299 (1979)). For approaches such as the one described in WO 06/050433, miRNA may be enriched via Poly(A)⁺ RNA selection methods. Poly(A)+ RNA is selected by selection with oligo-dT cellulose (see Sambrook et al., MOLECULAR CLONING—A LABORATORY MANUAL (2ND ED.), Vols. 1-3, Cold Spring Harbor Laboratory, Cold Spring Harbor, N.Y. (1989). Alternatively, separation of RNA from DNA can be accomplished by organic extraction, for example, with hot phenol or phenol/chloroform/isoamyl alcohol.

If desired, RNAse inhibitors may be added to the lysis buffer. Likewise, for certain cell types, it may be desirable to add a protein denaturation/digestion step to the protocol.

The sample of RNA can comprise a plurality of different miRNA molecules, each different miRNA molecule having a different nucleotide sequence. In a specific embodiment, the miRNA molecules in the RNA sample comprise at least 100 different nucleotide sequences. More preferably, the mRNA molecules of the RNA sample comprise miRNA molecules corresponding to each of the informative miRNAs. In another specific embodiment, the RNA sample is a mammalian RNA sample.

In a specific embodiment, total RNA or miRNA from cells are used in the methods of the invention. The source of the RNA can be cells of a plant or animal, human, mammal, primate, non-human animal, dog, cat, mouse, rat, bird, yeast, eukaryote, prokaryote, etc. In specific embodiments, the method of the invention is used with a sample containing total miRNA or total RNA from 1×10⁶ cells or less.

Probes to the homologs of the miRNA sequences disclosed herein can be employed preferably wherein non-human nucleic acid is being assayed.

Methods of Using Bladder Cancer miRNA Sets

The present invention provides methods of using miRNA sets to analyze a sample from an individual so as to determine the classification, invasive or metastatic potential and/or survival risk of an individual's bladder cancer tumor at a molecular level, i.e., to determine a prognosis, stage classification and/or course of treatment for the individual from which the sample is obtained. The individual need not actually be having bladder cancer. Essentially, the expression of specific miRNA genes in the individual, or a sample taken therefrom, is analyzed, e.g., compared to a standard or control, to determine if the pattern of expression indicates a specific bladder cancer stage, a progressing or non-progressing character or propensity, a good or a poor prognosis for health and/or survival, a high or low likelihood of responding to treatment, an elevated or reduced risk of metastasis, etc. For example, assuming two bladder cancer-related conditions, X and Y, one can compare the levels of expression of bladder cancer prognostic miRNAs for condition X in an individual to the respective levels of the miRNA in a control, wherein the levels of expression in the control represent the levels of expression of the miRNAs exhibited by samples having condition X. In this instance, if the expression of the miRNAs in the individual's sample is substantially (i.e., statistically) similar to that of the control, then the individual is said to have condition X, whereas if the expression of the miRNAs in the individual's sample is substantially (i.e., statistically) different from that of the control, then the individual does not have condition X. Where, as here, the choice is bimodal (i.e., a sample is either X or Y), if the individual does not have condition X, the individual can additionally be said to have condition Y. For example, conditions X and Y can be a good prognosis and a poor prognosis, respectively, as defined by the particular disease or condition, such as bladder cancer, and the particular clinical status of the individual. Of course, the comparison to a control representing condition Y can also be performed. In this instance, if the expression of the miRNAs in the individual's sample is substantially (i.e., statistically) similar to that of the control, then the individual is said to have condition Y. Preferably both are performed simultaneously, such that each control acts as both a positive and a negative control. The distinguishing result may thus either be a demonstrable difference from the expression levels (i.e., the amount of miRNA or polynucleotides derived therefrom) represented by the control, or no significant difference.

Thus, in one embodiment, the method of determining a particular tumor-related status of an individual comprises the steps of (1) hybridizing labeled target polynucleotides from the individual to a microarray containing one of the above miRNA sets; (2) hybridizing standard or control polynucleotides molecules to the microarray, wherein the standard or control molecules are differentially labeled from the target molecules; and (3) determining the difference in transcript levels, or lack thereof, between the target and standard or control, wherein the difference, or lack thereof, determines the individual's tumor-related status. In a more specific embodiment, the standard or control molecules comprise miRNA-derived polynucleotides from a pool of samples from normal individuals, or a pool of tumor samples from individuals having sporadic-type tumors. In one embodiment, the standard or control (standard miRNA expression pattern) is an artificially-generated pool of miRNA-derived polynucleotides, which pool is designed to mimic the level of miRNA expression exhibited by clinical samples of normal or bladder cancer tumor tissue having a particular clinical indication (i.e., bladder cancer stage, presence of carcinoma-in-situ, presenting an elevated risk to survival, presenting an elevated risk of progressing, good prognosis or poor prognosis; no reoccurrence or metastasis within five years of initial diagnosis or reoccurrence or metastasis within five years of initial diagnosis; etc.). In another specific embodiment, the control molecules comprise a pool derived from normal or bladder cancer cell lines.

The present invention provides sets of miRNAs useful for detecting bladder cancer, distinguishing “good prognosis” from “poor prognosis” tumor types. In a selected embodiment, “good prognosis” means no presence of invasive stage bladder cancer (e.g., T2 or higher) or carcinoma-in-situ, no reoccurrence or metastasis, in the individual from which the sample was taken, within, e.g., one to five years of initial diagnosis, and “poor prognosis” means progression to an invasive bladder cancer stage, formation of carcinoma-in-situ, reoccurrence, metastasis or death within, e.g., one to five years of initial diagnosis. Thus, in one embodiment of the above method, the level of polynucleotides (i.e., miRNA or polynucleotides derived therefrom) in a sample from an individual, expressed from the different miRNAs provided in any of Table 3 and FIG. 1A-1C are compared to the level of expression of the same miRNAs from a control, wherein the control comprises miRNA-related polynucleotides derived from samples obtained from individuals with no bladder cancer, showing no progression to invasive stage bladder cancer, no presence of carcinoma-in-situ, free of reoccurrence or metastasis and/or surviving at, e.g., one to five years or more, samples taken from individuals having bladder cancer, showing progression to invasive stage bladder cancer, presence of carcinoma-in-situ, having reoccurrence or metastasis and/or death at, e.g., one to five years or more, or both. Preferably, the comparison is to both, and preferably the comparison is to polynucleotide pools from a number of non-invasive stage, non-carcinoma-in-situ or otherwise “good prognosis” and invasive stage, carcinoma-in-situ or otherwise “poor prognosis” samples, respectively. Where, for example, the individual's miRNA expression most closely resembles or correlates with the “good prognosis” control, and does not resemble or correlate with the “poor prognosis” control, the individual is classified as having a good prognosis. Where the pool is not pure “good prognosis” or “poor prognosis,” for example, a sporadic pool may be used. A set of experiments should be performed in which nucleic acids from individuals with known prognosis status are hybridized against the pool, in order to define the expression templates for the “good prognosis” and “poor prognosis” group. Nucleic acids from each individual with unknown prognosis status are hybridized against the same pool and the miRNA expression profile is compared to the template(s) to determine the individual's prognosis (or bladder cancer stage classification, carcinoma-in-situ status, etc.).

The control or standard may be presented in a number of different formats. For example, the control, or template, to which the expression of miRNA genes in a bladder cancer tumor sample is compared may be the average absolute level of expression of each of the miRNAs in a pool of miRNAs pooled from bladder cancer tumor samples obtained from a plurality of bladder cancer patients. In this case, the difference between the absolute level of expression of these miRNAs in the control and in a sample from a bladder cancer patient provides the degree of similarity or dissimilarity of the level of expression in the patient sample and the control. The absolute level of expression may be measured by the intensity of the hybridization of the nucleic acids to an array. In other embodiments, the values for the expression levels of the miRNAs in both the patient sample and control are transformed. For example, the expression level value for the patient, and the average expression level value for the pool, for each of the miRNA genes selected, may be transformed by taking the logarithm of the value. Moreover, the expression level values may be normalized by, for example, dividing by the median hybridization intensity of all of the samples that make up the pool. The control may be derived from hybridization data obtained simultaneously with the patient sample expression data, or may constitute a set of numerical values stores on a computer, or on computer-readable medium.

Poor prognosis of bladder cancer may indicate that a tumor is relatively aggressive, while good prognosis may indicate that a tumor is relatively nonaggressive. Therefore, the invention provides for a method of determining a course of treatment of a bladder cancer patient, comprising determining whether the level of expression of at least 2 of the different miRNAs listed in any of Table 3 and FIGS. 1A-1C, or one or more subsets thereof, correlates with the level of these miRNAs in a sample representing a good prognosis expression pattern or a poor prognosis pattern; and determining a course of treatment, wherein if the expression correlates with the poor prognosis pattern, the tumor is treated as an aggressive tumor.

Alternatively, all miRNAs disclosed herein may be used, i.e., all 70 informative miRNAs identified herein. In other embodiments, subsets of the miRNAs may be used. In a selected embodiment, the risk of bladder cancer progression in an individual is determined using the miRNAs listed in any of Table 3 and FIGS. 1A-1C. In another selected embodiment, the individual is identified as having Ta or T1 stage bladder cancer, and the prognosis of an individual is determined using the miRNAs listed in any of Table 3 and FIGS. 1A-1C. An individual may be identified as having bladder cancer and/or having Ta or T1 stage bladder cancer by an acceptable means (e.g., histology).

The similarity between the miRNA expression pattern of an individual and that of a control or template can be assessed in a number of ways. In the simplest case, the profiles can be compared visually in a printout of expression difference data. Alternatively, the similarity can be calculated mathematically as known in the art.

In a specific embodiment of the above method, said first threshold similarity value and optionally other threshold similarity values are selected by a method comprising (a) rank ordering in descending order said bladder cancer tumor samples that compose said pool of tumor samples by the degree of similarity between the level of expression of said miRNAs in each of said tumor samples to the mean level of expression of the same miRNAs of the remaining tumor samples that compose said pool to obtain a rank-ordered list, said degree of similarity being expressed as a similarity value; (b) determining an acceptable number of false negatives in said classifying, wherein said false negatives are bladder cancer patients for whom the expression levels of said at least two of the different miRNAs for which miRNAs are listed in any of Table 3 and FIGS. 1A-1C in said cell sample predicts that said patient will not have progressing bladder cancers within the chosen timeframe (e.g., one to five years or more) after initial diagnosis, but who has had progression of bladder cancer within a chosen timeframe after initial diagnosis; (c) determining a similarity value above which in said rank ordered list fewer than said acceptable number of tumor samples are false negatives; and (d) selecting said similarity value determined in step (c) as said first threshold similarity value; and (e) optionally selecting a second similarity value, optionally greater than said first similarity value, as a second threshold similarity value, etc.

In an even more specific embodiment of this method, said optional second threshold similarity value is selected in step (e) by a method comprising determining which of said tumor samples, taken from patients having progressing bladder cancer within set timeframe of initial diagnosis, in said rank ordered list has the greatest similarity value, and selecting said greatest similarity value as said second threshold similarity value. In even more specific embodiments, said first (and optionally second, etc. threshold similarity values) are correlation coefficients, and said first threshold similarity value is 0.4 and said second threshold similarity value is greater than 0.4. In another specific embodiment, said first similarity value is a similarity value above which at most 10% false negatives are predicted in a training set of tumors, and said second correlation coefficient is a coefficient above which at most 5% false negatives are predicted in said training set of tumors. In another specific embodiment, said first correlation coefficient is a coefficient above which 10% false negatives are predicted in a training set of tumors, and said second correlation coefficient is a coefficient above which no false negatives are predicted in said training set of tumors. In the above and other embodiments, “false negatives” are patients classified by the expression of the miRNAs as having a good prognosis, or who are predicted by such expression to have a good prognosis, but who actually do develop progressing bladder cancer within a selected timeframe.

In a more specific embodiment, the similarity value is the degree of difference between the absolute (i.e., untransformed) level of expression of each of the miRNAs in a tumor sample taken from a bladder cancer patient and the mean absolute level of expression of the same miRNAs in a control. In another more specific embodiment, the similarity value is calculated using expression level data that is transformed. In another more specific embodiment, the similarity value is expressed as a similarity metric, such as a correlation coefficient, representing the similarity between the level of expression of the miRNAs in the tumor sample and the mean level of expression of the same miRNAs in a plurality of bladder cancer tumor samples taken from bladder cancer patients.

In another specific embodiment, said first and optional second, etc. similarity values are derived from control expression data obtained in the same hybridization experiment as that in which the patient expression level data is obtained. In another specific embodiment, said first and second similarity values are derived from an existing set of expression data. In a more specific embodiment, said first and second correlation coefficients are derived from a mathematical sample pool. For example, comparison of the expression of miRNAs in new tumor samples may be compared to the pre-existing template determined for these miRNAs for patients in a previous study; the template, or average expression levels of each of the miRNAs can be used as a reference or control for any tumor sample. Preferably, the comparison is made to a template comprising the average expression level of at least two of the different miRNAs listed in any of Table 3 and FIGS. 1A-1C for the subjects (see Examples) clinically determined to have a non-progressing bladder cancer. The coefficient of correlation of the level of expression of these miRNAs in the tumor sample to the “non-progressing” patient template is then determined to produce a tumor correlation coefficient. For this control patient set, two similarity values may be derived: a first correlation coefficient that minimizes Type 1 and Type 2 error, and a second correlation coefficient that is higher than the first correlation coefficient. The second correlation coefficient is that of the actual progressing sample in the rank-ordered list of samples having the highest correlation to the non-progressing template.

Because the above methods may utilize arrays to which fluorescently-labeled miRNA and miRNA-derived target nucleic acids are hybridized, the invention also provides a method of classifying a bladder cancer patient according to prognosis, e.g., a bladder cancer patient, comprising the steps of (a) contacting first nucleic acids derived from a tumor sample taken from said bladder cancer patient, and second nucleic acids derived from two or more tumor samples from bladder cancer patients who have had no distant metastasis within the chosen period (one to five years of more) of initial diagnosis, with an array under conditions such that hybridization can occur, detecting at each of a plurality of discrete miRNA-specific probes on said array a first fluorescent emission signal from said first nucleic acids and a second fluorescent emission signal from said second nucleic acids that are bound to said array under said conditions, wherein said array comprises at least two of the different miRNAs listed in any of Table 3 and FIGS. 1A-1C and wherein at least 50% of the probes on said array are listed in Table 3 and FIGS. 1A-1C; (b) calculating the similarity between said first fluorescent emission signals and said second fluorescent emission signals across said at least two miRNAs; and (c) classifying said bladder cancer patient according to prognosis of his or her bladder cancer based on the similarity between said first fluorescent emission signals and said second fluorescent emission signals across said at least two miRNAs.

The use of miRNA sets is not restricted to the prognosis of bladder cancer-related conditions, and may be applied in a variety of phenotypes or conditions, clinical or experimental, in which miRNA expression plays a role. Where a set of miRNAs has been identified that corresponds to two or more phenotypes, the miRNA set can be used to distinguish these phenotypes. For example, the phenotypes may be the diagnosis and/or prognosis of clinical states or phenotypes associated with other cancers, other disease conditions, or other physiological conditions, wherein the expression level data is derived from a set of miRNAs correlated with the particular physiological or disease condition.

Further, the expression of miRNAs specific to other types of cancer may be used to differentiate patients or patient populations for those cancers for which different therapeutic regimens are indicated.

Predictive use of measured miRNA levels may also employ tools used to identify predictive miRNAs from test samples. For example, analysis of microarray data generated during implementation an array of miRNAs assembled as a classifier can employ SAM (Significance Analysis of Microarrays) implemented in TIGR MEV 3.1 software. For such analyses, 500 permutations of the data can also be performed in order to select differentially expressed miRNA controlled for false discovery rate estimations. In such predictive analyses, an average FDR of 0%, 2%, 5%, 10%, 20% or more can be set as a cut-off. miRNAs can be clustered hierarchically using Cluster 2.0. miRNAs can be assessed as median centered and normalized before clustering. Treeview 2.0 can be used for visualization.

Improving Sensitivity to miRNA Expression Level Differences

In using the miRNAs disclosed herein, and, indeed, using any sets of miRNAs to differentiate an individual having one phenotype from another individual having a second phenotype, one can compare the absolute expression of each of the miRNAs in a sample to a control; for example, the control can be the average level of expression of each of the miRNAs, respectively, in a pool of individuals. To increase the sensitivity of the comparison, however, the expression level values are preferably transformed in a number of ways.

For example, the expression level of each of the miRNAs can be normalized by the average expression level of all miRNAs the expression level of which is determined, or by the average expression level of a set of control miRNAs. Thus, in one embodiment, the miRNAs are represented by probes on a microarray, and the expression level of each of the miRNAs is normalized by the mean or median expression level across all of the miRNAs represented on the microarray. In a specific embodiment, the normalization is carried out by dividing the median or mean level of expression of all of the miRNAs on the microarray. In another embodiment, the expression levels of the miRNAs are normalized by the mean or median level of expression of a set of control miRNAs. In a specific embodiment, the control miRNAs comprise a set of miRNAs of known stable expression (e.g., “housekeeping” miRNAs). In another specific embodiment, the normalization is accomplished by dividing by the median or mean expression level of the control miRNAs.

The sensitivity of a miRNA-based assay will also be increased if the expression levels of individual miRNAs are compared to the expression of the same miRNAs in a pool of samples. Preferably, the comparison is to the mean or median expression level of each the miRNA miRNAs in the pool of samples. Such a comparison may be accomplished, for example, by dividing by the mean or median expression level of the pool for each of the miRNAs from the expression level each of the miRNAs in the sample. This has the effect of accentuating the relative differences in expression between miRNAs in the sample and miRNAs in the pool as a whole, making comparisons more sensitive and more likely to produce meaningful results that the use of absolute expression levels alone. The expression level data may be transformed in any convenient way; preferably, the expression level data for all is log transformed before means or medians are taken.

In performing comparisons to a pool, two approaches may be used. First, the expression levels of the miRNAs in the sample may be compared to the expression level of those miRNAs in the pool, where nucleic acid derived from the sample and nucleic acid derived from the pool are hybridized during the course of a single experiment. Such an approach requires that new pool nucleic acid be generated for each comparison or limited numbers of comparisons, and is therefore limited by the amount of nucleic acid available.

Alternatively (in certain embodiments, preferably), the expression levels in a pool, whether normalized and/or transformed or not, are stored on a computer, or on computer-readable media, to be used in comparisons to the individual expression level data from the sample (i.e., single-channel data).

The use of miRNA sets is not restricted to the prognosis of bladder cancer-related conditions, and may be applied in a variety of phenotypes or conditions, clinical or experimental, in which miRNA expression plays a role. Where a set of miRNAs has been identified that corresponds to two or more phenotypes, the miRNA set can be used to distinguish these phenotypes. For example, the phenotypes may be the diagnosis and/or prognosis of clinical states or phenotypes associated with other cancers, other disease conditions, or other physiological conditions, wherein the expression level data is derived from a set of miRNAs correlated with the particular physiological or disease condition.

Further, the expression of miRNAs specific to other types of cancer may be used to differentiate patients or patient populations for those cancers for which different therapeutic regimens are indicated.

Determination of miRNA Expression Levels

The expression levels of the miRNA in a sample may be determined by any means known in the art. The expression level may be determined by isolating and determining the level (i.e., amount) of each miRNA. Alternatively, or additionally, the level of specific post-transcriptional forms (e.g., processed forms) of miRNA may be determined.

Measuring the level of one or more miRNAs can be achieved using a variety of techniques that are well known to those of skill in the art. Such methods include, but are not limited to, e.g., approaches that employ miRNA-specific probes (including, but not limited to, array-based detection of miRNAs; quantitative or semi-quantitative RT-PCR; Northern blot analysis (including art-recognized approaches specifically adapted to detect small RNAs); solution hybridization detection; approaches that involve generation of larger detection structures, e.g., as described in US 2005/0074788; and approaches such as those described in WO 07/081,720.

The level of expression of specific miRNAs can be accomplished by determining the amount of miRNA, or polynucleotides derived therefrom, present in a sample. Any method for determining RNA levels can be used. For example, RNA is isolated from a sample and separated on an agarose gel. The separated RNA is then transferred to a solid support, such as a filter. Nucleic acid probes representing one or more miRNAs are then hybridized to the filter by northern hybridization, and the amount of miRNAs are determined. Such determination can be visual, or machine-aided, for example, by use of a densitometer. Another method of determining RNA levels is by use of a dot-blot or a slot-blot. In this method, RNA, or nucleic acid derived therefrom, from a sample is labeled. The RNA or nucleic acid derived therefrom is then hybridized to a filter containing oligonucleotides derived from one or more miRNAs, wherein the oligonucleotides are placed upon the filter at discrete, easily-identifiable locations.

Hybridization, or lack thereof, of the labeled RNA to the filter-bound oligonucleotides is determined visually or by densitometer. Polynucleotides can be labeled using a radiolabel or a fluorescent (i.e., visible) label. These examples are not intended to be limiting; other methods of determining RNA abundance are known in the art.

### Microarrays

In selected embodiments, polynucleotide microarrays are used to measure expression so that the expression status of each of the miRNAs above is assessed simultaneously. In a specific embodiment, the invention provides oligonucleotide arrays comprising probes hybridizable to the miRNAs corresponding to each of the miRNA sets described above (i.e., miRNAs to distinguish progressing from non-progressing bladder cancers). In a more specific embodiment, the invention provides oligonucleotide arrays comprising probes having sequences identified by sequences complementary to miRNAs listed in Table 3 and FIGS. 1A-1C, or a subset or subsets of at least 10, 20, 30, 40, 50, 60 or 75 of these probes.

In specific embodiments, the invention provides polynucleotide arrays in which polynucleotide probes complementary and hybridizable to the bladder cancer prognosis-related miRNAs described herein are at least 50%, 60%, 70%, 80%, 85%, 90%, 95% or 98% of the probes on said array. In another specific embodiment, the microarray of the invention comprises probes to at least 5 miRNAs selected from Table 3 and FIGS. 1A-1C. The microarray may comprise probes complementary and hybridizable to 10%, 15%, 20%, 25%, 30%, 40%, 50%, 60%, 70%, 80%, or 90% of the different miRNAs listed in any of Table 3 and FIGS. 1A-1C.

### Treatment of Cancer

In certain embodiments, a subject or patient is administered with a therapeutically effective dose of an anti-neoplastic (anti-cancer) agent, simultaneously, before or after assessment of miRNA expression as described infra. Therapeutically effective dosages of many anti-neoplastic agents are well-established, and can be found, for example, in Cancer Chemotherapy and Biotherapy: A Reference Guide Edition Number:2 Tenenbaum, ed. Saunders & CO (1994), which is incorporated herein by reference.

Also provided herein are methods for treating a neoplastic disorder in a subject in need thereof. Exemplary anti-neoplastic agents include 1,3-Bis(2-Chloroethyl)-1-NitrosoUrea (BCNU), Busulfan, Carboplatin, Carmustine, Chlorambucil, Cisplatin, Cyclophosphamide, Dacarbazine, Daunorubicin, Doxorubicin, Epirubicin, Etoposide, Idarubicin, Ifosfamide, Irinotecan, Lomustine, Mechlorethamine, Melphalan, Mitomycin C, Mitoxantrone, Oxaliplatin, Temozolomide, and Topotecan and ionizing radiation.

The efficacy of compositions disclosed herein in preventing or treating neoplastic disorders can be tested, for example, in animal models of specific neoplastic disorders. Numerous examples of animal models are well known to those skilled in the art, and are disclosed, for example, in Holland, Mouse Models of Cancer (Wiley-Liss 2004); Teicher, Tumor Models in Cancer Research (Humana Press; 2001); Kallman, Rodent Tumor Models in Experimental Cancer Therapy (Mcgraw-Hill, TX, 1987); Hedrich, The Laboratory Mouse (Handbook of Experimental Animals) (Academic Press, 2004); and Arnold and Kopf-Maier, Immunodeficient Animals: Models for Cancer Research (Contributions to Oncology, Vol 51) (Karger, 1996), the contents of which are incorporated herein in their entirety.

### Pharmaceutical Compositions

In certain embodiments, the present invention provides a pharmaceutical composition for treating an individual by administration of a nucleic acid agent or drug, wherein the composition comprises a therapeutically effective amount of a nucleic acid or drug according to the present invention. The pharmaceutical composition may be for human or animal usage. Typically, a physician will determine the actual dosage which will be most suitable for an individual subject and it will vary with the age, weight and response of the particular patient.

The composition may optionally comprise a pharmaceutically acceptable carrier, diluent, excipient or adjuvant. The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s), and other carrier agents that may aid or increase the viral entry into the target site (such as for example a lipid delivery system).

Where appropriate, the pharmaceutical compositions can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavemosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

### Kits

In certain embodiments, the invention provides kits which contain, e.g., compositions of the invention as described herein and/or components specifically useful in the methods described herein. In other embodiments, the invention provides kits useful in the treatment of individuals diagnosed with bladder cancer, in certain embodiments diagnosed using the methods of the invention. For example, the bladder cancer detection, classification and/or prognostic agents of the invention, or the pharmaceutical compositions of the invention, can be included in a container, pack, or dispenser together with instructions for use.

### Recombinant Polynucleotide and Polypeptide Expression

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Materials and Methods

### Patient Material

Biological material from incident and in some cases recurrent tumors was obtained directly from surgery after removal of the necessary amount of tissue for routine pathology examination. All tumors were graded by the Bergkvist classification system. Informed written consent was obtained from all patients, and research protocols were approved by the ethical committee of Aarhus County. The tumors were frozen at −80° C. in a guanidinium thiocyanate solution and RNA was extracted from the samples using a standard Trizol RNA extraction method (Invitrogen). All RNA was quality controlled using an Agilent Bioanalyzer (criteria: 28S/18S>1 and RIN>5).

Microarray miRNA Expression Profiling and Data Analysis

Microarrays were produced using a LNA based oligonucleotide probe library (mercury LNA array ready to spot v.7.1) purchased from Exiqon (Denmark). Oligonucleotides were spotted in duplicates in a 10 uM phosphate buffer on CodeLink microarray glass slides (GE-Health Care) using a VersArray Chipwriter Pro system (Biorad) as previously described (Dyrskjot et al. (2005) Clin Cancer Res 11: 4029-4036). Two μg of sample RNA was directly labeled with Hy3 using the mercury LNA array labeling kit (Exiqon). As reference, a pool of RNA extracted from bladder-, prostate-, and colon tumors was used. For each experiment 2 μg of the reference RNA was labeled with Hy5 using the LNA array labeling kit (Exiqon). Hybridization and washing of the microarray slides were performed as described by the Exiqon. Scanning was performed using a ScanArray 4000 (manufactor). Following scanning of the microarrays, TIGR spotfinder 2.23 software was used to generate raw-intensity data, which were LOWESS (global) normalized using TIGR MIDAS 2.19 software (Saeed et al. (2003) Biotechniques 34: 374-378). Average log₂ ratios were calculated from the normalized data based on the 2 measurements of each miRNA. For analysis of microarray data, SAM (Significance Analysis of Microarrays) implemented in TIGR MEV 3.1 software was used (Ibid). For all analyses, 500 permutations of the data were performed in order to select differentially expressed miRNA controlled for false discovery rate estimations. In all analyses, an average FDR of 0% was used as a cut-off miRNAs were clustered hierarchically using Cluster 2.0. miRNAs were median centered and normalized before clustering. Treeview 2.0 was used for visualization.

QPCR miRNA Expression Profiling

Taqman PCR assays (Applied Biosystems) were used for measuring miRNA expression quantitatively. PCR assays were performed as described by the manufactor using an ABI7900 PCR system. For normalization, miRNAs were tested that showed minimal variation (let-7f, let-7d and miR-296) in the microarray dataset using Normfinder (Andersen et al. (2004) Cancer Res 64: 5245-5250); together with standard normalization ribosomal RNA (RNU06B, RNU43 and Z30).

In Situ Detection of miRNAs

In situ detection of microRNAs was performed upon 10 frozen tissue sections from surgery material. Sections were fixed in 4% paraformaldehyde, acetylated and pre-hybridized in hybridization solution (50% formamide, 5×SSC, 0.5 mg/ml yeast tRNA, 1×Denhardt's solution) for 30 minutes prior to hybridization. 3 pmol probe (LNA-modified and FITC-labelled oligonucleotide, Exiqon) complementary to miR-21, miR-145 and miR-129 were hybridized to the sections for 1 hr at 25° C. lower than Tm of the probe. After post-hybridization washes, in situ hybridization signals were detected using the tyramide signal amplification system (Perkin Elmer) according to the manufacturer's instructions. Slides were mounted in Prolong Gold containing DAPI (Invitrogen) and analyzed with an Olympus MVX10 microscope equipped with a CCD camera and Olympus CellP software.

The expression of 315 unique human miRNA genes in: 11 biopsies of normal urothelium obtained from healthy individuals; 27 Ta tumors (9 progressed to T2-4; 33%); 40 T1 tumors (20 progressed to T2-4; 50%); and 27 T2-4 tumors was assessed using LNA based oligonucleotide microarrays. Patients with non-progressing Ta and T1 tumors had a median follow-up time of 62 months, while patients with progressing tumors had a median time to progression of 10 months. Clinical and histopathological parameters for the patients included in this study are listed in Table 1.

TABLE 1
Clinical and Histopathological Parameters for Patients Included in this Study

| | | | | | | | | | Death cause | | |
|---|---|---|---|---|---|---|---|---|---|---|---|
| | | | | | | | | | (0 = alive, | | |
| | | | | | Progression | | | | 1 = died from | | |
| | | | | | (0 no | | | | bladder cancer, | | |
| | | | | | progression, | | Progression | | 2 = other cause, | | |
| | | | | CIS | 1 prog to T1, | | free | Follow-up | 3 unknown | | |
| Material | Sample # | Stage | Grade | diagnosis | 2 prog to T2+) | Follow-up* | survival | (total) | cause | SEX | AGE |
| Normal | \acute{ }9-99 | — | — | — | — | — | — | — | — | — | — |
| Normal | 316-99 | — | — | — | — | — | — | — | — | — | — |
| Normal | 335-99 | — | — | — | — | — | — | — | — | — | — |
| Normal | 337-99 | — | — | — | — | — | — | — | — | — | — |
| Normal | 340-99 | — | — | — | — | — | — | — | — | — | — |
| Normal | 341-99 | — | — | — | — | — | — | — | — | — | — |
| Normal | 343-99 | — | — | — | — | — | — | — | — | — | — |
| Normal | 344-99 | — | — | — | — | — | — | — | — | — | — |
| Normal | 345-99 | — | — | — | — | — | — | — | — | — | — |
| Normal | 55-99 | — | — | — | — | — | — | — | — | — | — |
| Normal | 70-99 | — | — | — | — | — | — | — | — | — | — |
| Tumor | 1058-2 | Ta | 2 | no cis | 2 | 42 | 42 | 43 | 1 | F | 67 |
| Tumor | 1093-1 | Ta | 3 | no cis | 2 | 66 | 66 | 77 | 1 | M | 81 |
| Tumor | 1415-1 | Ta | 2 | no cis | 2 | 28 | 25 | 31 | 1 | M | 65 |
| Tumor | 1828-1 | Ta | 3 | no CIS | 2 | 7 | 7 | 7 | 0 | M | 78 |
| Tumor | 217-11 | Ta | 2 | no cis | 2 | 61 | 61 | 71 | 0 | M | 51 |
| Tumor | 242-3 | Ta | 3 | CIS | 2 | 12 | 12 | 15 | 1 | M | 74 |
| Tumor | 846-1 | Ta | 2 | no CIS | 2 | 11 | 4 | 11 | 1 | F | 72 |
| Tumor | 856-2 | Ta | 2 | CIS | 2 | 47 | 1 | 47 | 1 | M | 75 |
| Tumor | 992-1 | Ta | 2 | no cis | 2 | 20 | 18 | 21 | 1 | M | 67 |
| Tumor | 1327-1 | Ta | 2 | no cis | 1 | 22 | 22 | 26 | 2 | F | 67 |
| Tumor | 1354-1 | Ta | 3 | no cis | 1 | 12 | 23 | 12 | 1 | M | 80 |
| Tumor | 1656-1 | Ta | 3 | no cis | 1 | 12 | 12 | 15 | 0 | F | 80 |
| Tumor | 1066-1 | Ta | 3 | no cis | 0 | 67 | 67 | 69 | 0 | M | 80 |
| Tumor | 1077-1 | Ta | 2 | no cis | 0 | 62 | 62 | 63 | 0 | M | 66 |
| Tumor | 1105-1 | Ta | 2 | no cis | 0 | 31 | 31 | 53 | 0 | M | 57 |
| Tumor | 1335-1 | Ta | 2 | CIS | 0 | 42 | 42 | 43 | 0 | M | 48 |
| Tumor | 1350-1 | Ta | 3 | no cis | 0 | 54 | 54 | 54 | 0 | M | 84 |
| Tumor | 1352-1 | Ta | 2 | no cis | 0 | 38 | 38 | 41 | 0 | F | 43 |
| Tumor | 1408-1 | Ta | 1 | no cis | 0 | 36 | 36 | 38 | 0 | M | 33 |
| Tumor | 332-1 | Ta | 2 | no cis | 0 | 66 | 66 | 116 | 0 | M | 69 |
| Tumor | 521-1 | Ta | 1 | no cis | 0 | 46 | 46 | 105 | 0 | M | 27 |
| Tumor | 62-9 | Ta | 3 | CIS | 0 | 44 | 44 | 52 | 3 | M | 70 |
| Tumor | 669-1 | Ta | 2 | no cis | 0 | 95 | 95 | 95 | 0 | M | 59 |
| Tumor | 686-1 | Ta | 2 | no cis | 0 | 81 | 81 | 81 | 0 | M | 74 |
| Tumor | 833-2 | Ta | 3 | no cis | 0 | 73 | 73 | 73 | 0 | F | 44 |
| Tumor | 876-1 | Ta | 2 | no cis | 0 | 85 | 85 | 87 | 0 | M | 57 |
| Tumor | 997-1 | Ta | 2 | no cis | 0 | 53 | 53 | 69 | 0 | M | 52 |
| Tumor | 1017-1 | T1 | 3 | CIS | 2 | 42 | 9 | 52 | 1 | M | 79 |
| Tumor | 1047-1 | T1 | 3 | CIS | 2 | 14 | 6 | 17 | 1 | M | 76 |
| Tumor | 1056-1 | T1 | 3 | CIS | 2 | 51 | 25 | 60 | 1 | F | 58 |
| Tumor | 1082-1 | T1 | 3 | CIS | 2 | 69 | 69 | 76 | 0 | F | 81 |
| Tumor | 1134-1 | T1 | 3 | no cis | 2 | 28 | 25 | 43 | 1 | M | 81 |
| Tumor | 1224-1 | T1 | 3 | CIS | 2 | 25 | 11 | 26 | 1 | F | 73 |
| Tumor | 1252-1 | T1 | 3 | CIS | 2 | 58 | 58 | 60 | 1 | M | 66 |
| Tumor | 1336-1 | T1 | 3 | no cis | 2 | 54 | 40 | 58 | 0 | M | 77 |
| Tumor | 1425-1 | T1 | 3 | ND | 2 | 26 | 2 | 26 | 0 | M | 70 |
| Tumor | 1443-1 | T1 | 3 | ND | 2 | 10 | 10 | 53 | 0 | M | 69 |
| Tumor | 1456-1 | T1 | 3 | no cis | 2 | 19 | 19 | 32 | 1 | F | 72 |
| Tumor | 1571-1 | T1 | 3 | CIS | 2 | 2 | 2 | 9 | 1 | M | 68 |
| Tumor | 1660-1 | T1 | 3 | ND | 2 | 2 | 2 | 8 | 0 | M | 80 |
| Tumor | 1887-1 | T1 | 3 | no cis | 2 | 2 | 1 | 11 | 0 | F | 67 |
| Tumor | 247-9 | T1 | 3 | CIS | 2 | 15 | 15 | 18 | 1 | M | 81 |
| Tumor | 320-7 | T1 | 3 | CIS | 2 | 8 | 8 | 10 | 1 | M | 69 |
| Tumor | 365-1 | T1 | 3 | no CIS | 2 | 17 | 7 | 17 | 1 | M | 59 |
| Tumor | 886-1 | T1 | 3 | no cis | 2 | 5 | 5 | 10 | 1 | M | 76 |
| Tumor | 938-1 | T1 | 3 | ND | 2 | 0 | 7 | 7 | 1 | M | 83 |
| Tumor | 1725-1 | T1 | 3 | no cis | 2 | 4 | 4 | 4 | 3 | M | 62 |
| Tumor | 1010-1 | T1 | 3 | CIS | 0 | 77 | 77 | 82 | 0 | F | 69 |
| Tumor | 1031-1 | T1 | 3 | CIS | 0 | 70 | 70 | 81 | 0 | M | 68 |
| Tumor | 1034-2 | T1 | 3 | CIS | 0 | 70 | 70 | 79 | 0 | F | 74 |
| Tumor | 1065-1 | T1 | 3 | no cis | 0 | 68 | 68 | 79 | 0 | M | 83 |
| Tumor | 1073-1 | T1 | 3 | no cis | 0 | 72 | 72 | 76 | 0 | F | 65 |
| Tumor | 1182-1 | T1 | 3 | CIS | 0 | 64 | 64 | 68 | 0 | M | 45 |
| Tumor | 1280-1 | T1 | 3 | no cis | 0 | 47 | 47 | 62 | 0 | M | 62 |
| Tumor | 1293-1 | T1 | 3 | no cis | 0 | 45 | 45 | 61 | 0 | M | 64 |
| Tumor | 1375-1 | T1 | 2 | no cis | 0 | 39 | 39 | 52 | 0 | M | 53 |
| Tumor | 1482-1 | T1 | 3 | no cis | 0 | 37 | 37 | 41 | 0 | M | 67 |
| Tumor | 177-1 | T1 | 3 | no cis | 0 | 67 | 67 | 133 | 0 | M | 66 |
| Tumor | 684-4 | T1 | 3 | CIS | 0 | 73 | 73 | 81 | 0 | M | 77 |
| Tumor | 735-1 | T1 | 3 | no cis | 0 | 95 | 95 | 95 | 0 | M | 69 |
| Tumor | 760-1 | T1 | 3 | CIS | 0 | 90 | 90 | 101 | 0 | M | 72 |
| Tumor | 845-1 | T1 | 3 | no cis | 0 | 62 | 62 | 62 | 0 | F | 78 |
| Tumor | 855-1 | T1 | 3 | no cis | 0 | 56 | 56 | 56 | 0 | M | 64 |
| Tumor | 881-1 | T1 | 3 | no cis | 0 | 83 | 83 | 83 | 0 | M | 70 |
| Tumor | 927-1 | T1 | 3 | no cis | 0 | 87 | 87 | 88 | 0 | M | 71 |
| Tumor | 998-1 | T1 | 3 | no cis | 0 | 57 | 57 | 81 | 0 | M | 53 |
| Tumor | 1022-1 | T1 | 3 | no cis | 0 | 56 | 56 | 80 | 0 | M | 61 |
| Tumor | 1574-1 | T2-4 | 3 | — | — | — | — | 5 | 1 | F | 72 |
| Tumor | 1015-1 | T2-4 | 3 | — | — | — | — | 30 | 1 | M | 58 |
| Tumor | 1041-1 | T2-4 | 3 | — | — | — | — | 357 | 0 | M | 62 |
| Tumor | 1044-1 | T2-4 | 3 | — | — | — | — | 38 | 1 | M | 55 |
| Tumor | 1055-1 | T2-4 | 3 | — | — | — | — | 350 | 0 | M | 70 |
| Tumor | 1113-5 | T2-4 | 2 | — | — | — | — | 160 | 0 | F | 62 |
| Tumor | 1154-1 | T2-4 | 2 | — | — | — | — | 36 | 1 | M | 60 |
| Tumor | 1167-1 | T2-4 | 4 | — | — | — | — | 95 | 1 | F | 51 |
| Tumor | 1178-1 | 12-4 | 3 | — | — | — | — | 28 | 1 | M | 68 |
| Tumor | 1271-1 | T2-4 | 3 | — | — | — | — | 47 | 1 | F | 58 |
| Tumor | 1285-1 | T2-4 | 3 | — | — | — | — | 276 | 0 | M | 49 |
| Tumor | 1321-1 | T2-4 | ? | — | — | — | — | 33 | 1 | F | 51 |
| Tumor | 1385-1 | T2-4 | 2 | — | — | — | — | 243 | 1 | M | 67 |
| Tumor | 1485-1 | T2-4 | 4 | — | — | — | — | 43 | 1 | M | 65 |
| Tumor | 1530-1 | T2-4 | 4 | — | — | — | — | 37 | 1 | M | 74 |
| Tumor | 1663-1 | T2-4 | 3 | — | — | — | — | 31 | 1 | M | 56 |
| Tumor | 1682-1 | T2-4 | 3 | — | — | — | — | 42 | 1 | F | 66 |
| Tumor | 217-6 | T2-4 | 2 | — | — | — | — | 36 | 1 | F | 51 |
| Tumor | 472-1 | T2-4 | 3 | — | — | — | — | 14 | 1 | F | 63 |
| Tumor | 523-1 | T2-4 | 3 | — | — | — | — | 57 | 1 | M | 66 |
| Tumor | 575-1 | T2-4 | 3 | — | — | — | — | 83 | 1 | M | 53 |
| Tumor | 621-5 | T2-4 | 2 | — | — | — | — | 76 | 1 | M | 59 |
| Tumor | 724-1 | T2-4 | 3 | — | — | — | — | 420 | 0 | M | 53 |
| Tumor | 752-3 | T2-4 | 3 | — | — | — | — | 190 | 1 | M | 51 |
| Tumor | 805-1 | T2-4 | 3 | — | — | — | — | 62 | 1 | M | 73 |
| Tumor | 882-1 | T2-4 | 4 | — | — | — | — | 77 | 1 | F | 55 |
| Tumor | 981-1 | T2-4 | 3 | — | — | — | — | 28 | 1 | M | 63 |

*Months from tumor to last visit to the clinic, or to cystectomy.

### Example 1

### miRNAs Differentially Expressed in Normal Bladder and in Bladder Tumors

Initially, miRNAs differentially expressed between normal urothelium, Ta, T1 and T2-4 stages were identified using SAM by performing 500 permutations and using an average FDR of 0% as cut-off level. The 80 probes against human miRNAs that showed significant differential expressions are listed in FIG. 1A. Exiqon probe ID numbers corresponding to those miRNAs shown in FIG. 1A are presented below in Table 2.

TABLE 2
Exiqon Probe IDs Corresponding to FIG. 1A miRNAs.

| miRNA | probe ID |
|---|---|
| hsa-miR-494 | 11126 |
| hsa-miR-500 | 11132 |
| hsa-miR-527 | 11177 |
| hsa-miR-525 | 11175 |
| hsa-miR-520a | 11165 |
| hsa-miR-519d | 11163 |
| hsa-miR-492 | 11124 |
| hsa-miR-519e* | 13132 |
| hsa-miR-520d* | 13134 |
| hsa-miR-516-5p | 11151 |
| hsa-miR-526b | 11176 |
| hsa-miR-510 | 11142 |
| hsa-miR-518c* | 13131 |
| hsa-miR-185 | 5560 |
| hsa-miR-184 | 10978 |
| hsa-miR-198 | 10993 |
| hsa-miR-193b | 10987 |
| hsa-miR-328 | 11060 |
| hsa-miR-489 | 11121 |
| hsa-miR-483 | 13180 |
| hsa-miR-526c | 13137 |
| hsa-miR-320 | 11054 |
| hsa-miR-373* | 11086 |
| hsa-miR-498 | 11130 |
| hsa-miR-503 | 11135 |
| hsa-miR-129 | 10934 |
| hsa-miR-133b | 10939 |
| hsa-miR-296 | 11035 |
| hsa-miR-150 | 10958 |
| hsa-miR-151 | 10959 |
| hsa-miR-144 | 10950 |
| hsa-miR-199a | 10994 |
| hsa-miR-23a | 11026 |
| hsa-miR-26a | 11030 |
| hsa-miR-126* | 10930 |
| hsa-miR-455 | 13179 |
| hsa-miR-143 | 13177 |
| hsa-miR-145 | 10951 |
| hsa-miR-199b | 10996 |
| hsa-miR-199a* | 10995 |
| hsa-miR-125b | 10929 |
| hsa-miR-125a | 10928 |
| hsa-miR-21 | 5740 |
| hsa-miR-338 | 11067 |
| hsa-miR-302b | 11043 |
| hsa-miR-302a* | 11042 |
| hsa-miR-29c | 11041 |
| hsa-miR-29b | 11040 |
| hsa-miR-29a | 11039 |
| hsa-miR-126 | 4610 |
| hsa-miR-34b | 11073 |
| hsa-miR-30b | 11049 |
| hsa-miR-324-5p | 11057 |
| hsa-miR-212 | 11012 |
| hsa-miR-202 | 11003 |
| hsa-miR-20a | 10999 |
| hsa-miR-22 | 11020 |
| hsa-miR-219 | 11019 |
| hsa-miR-200b | 11001 |
| hsa-miR-200c | 11002 |
| hsa-miR-203 | 11004 |
| hsa-miR-141 | 10946 |
| hsa-miR-200a | 11000 |
| hsa-miR-193a | 10986 |
| hsa-miR-191* | 13126 |
| hsa-miR-98 | 11182 |
| hsa-let-7b | 10912 |
| hsa-let-7g | 4500 |
| hsa-miR-18b | 13141 |
| hsa-miR-107 | 10923 |
| hsa-miR-127 | 10931 |

Several miRNAs were found to be specifically associated with normal, Ta tumors and T2-4 tumors; however, T1 tumors showed expression similarities to either the Ta or the T2-4 tumors. Accordingly, it was not possible to identify miRNAs associated with stage T1 tumors only. Table 3 presents a list of miRNAs that were identified as the most significantly differentially expressed:

TABLE 3
Summary of the most significant differentially expressed miRNAs.

| Exiqon probe ID* | miRNA | Regulation | Fold change^{a} | p-values^{b} |
|---|---|---|---|---|
| | Normal vs tumor comparison^{c} | | | |
| 10986 | hsa-miR-193a | Up in tumor | 1.1 | 1.1E−07 |
| 11164 | hsa-miR-519e* | Up in tumor | 0.8 | 2.6E−07 |
| 5740 | hsa-miR-21 | Up in tumor | 1.9 | 3.6E−07 |
| 11003 | hsa-miR-202 | Up in tumor | 1.0 | 6.1E−07 |
| 10993 | hsa-miR-198 | Up in tumor | 1.1 | 2.6E−06 |
| 13179 | hsa-miR-455 | Down in tumor | −1.4 | 1.2E−09 |
| 10930 | hsa-miR-126* | Down in tumor | −1.0 | 1.6E−08 |
| 10951 | hsa-miR-145 | Down in tumor | −3.0 | 1.7E−08 |
| 13177 | hsa-miR-143 | Down in tumor | −1.4 | 8.6E−08 |
| 10929 | hsa-miR-125b | Down in tumor | −1.3 | 2.3E−07 |
| | Ta vs T2-4 comparison^{c} | | | |
| 10934 | hsa-miR-129 | Up in T2-4 | 1.4 | 8.6E−09 |
| 11135 | hsa-miR-503 | Up in T2-4 | 1.3 | 2.3E−08 |
| 11054 | hsa-miR-320 | Up in T2-4 | 1.0 | 4.5E−08 |
| 11130 | hsa-miR-498 | Up in T2-4 | 0.7 | 5.7E−08 |
| 13180 | hsa-miR-483 | Up in T2-4 | 1.2 | 3.3E−07 |
| 4500 | hsa-let-7g | Down in T2-4 | −0.7 | 2.0E−07 |
| 13141 | hsa-miR-18b | Down in T2-4 | −0.6 | 5.7E−07 |
| 11001 | hsa-miR-200b | Down in T2-4 | −1.0 | 1.1E−06 |
| 10946 | hsa-miR-141 | Down in T2-4 | −1.5 | 1.7E−06 |
| 11019 | hsa-miR-219 | Down in T2-4 | −1.1 | 2.3E−06 |
| | Progression vs no progression comparison | | | |
| 10934 | hsa-miR-129 | Up in progressing | 1.1 | 0.0001 |
| 10939 | hsa-miR-133b | Up in progressing | 0.3 | 0.00078 |
| 11054 | hsa-miR-320 | Up in progressing | 0.7 | 0.000853 |
| 11135 | hsa-miR-503 | Up in progressing | 0.8 | 0.00115 |
| 11086 | hsa-miR-373* | Up in progressing | 0.7 | 0.001326 |
| 13137 | hsa-miR-526c | Up in progressing | 0.6 | 0.001887 |
| 11130 | hsa-miR-498 | Up in progressing | 0.5 | 0.003027 |
| 13131 | hsa-miR-518c* | Up in progressing | 0.4 | 0.004759 |
| 5560 | hsa-miR-185 | Up in progressing | 0.2 | 0.007219 |
| 13180 | hsa-miR-483 | Up in progressing | 0.5 | 0.027381 |
| 10951 | hsa-miR-145 | Up in progressing | 0.8 | 0.064775 |
| 11041 | hsa-miR-29c | Down in progressing | −0.8 | 0.000416 |
| 11040 | hsa-miR-29b | Down in progressing | −0.7 | 0.001261 |
| 11039 | hsa-miR-29a | Down in progressing | −0.7 | 0.003252 |
| 11004 | hsa-miR-203 | Down in progressing | −0.9 | 0.006858 |
| | CIS vs no CIS comparison | | | |
| 10934 | hsa-miR-129 | Up in CIS | 1.1 | 4.89E−05 |
| 11135 | hsa-miR-503 | Up in CIS | 1.0 | 7.3E−05 |
| 11130 | hsa-miR-498 | Up in CIS | 0.6 | 0.000142 |
| 13180 | hsa-miR-483 | Up in CIS | 0.7 | 0.001529 |
| 11248 | hsa-miR-451 | Up in CIS | 0.9 | 0.001646 |
| 11086 | hsa-miR-373* | Up in CIS | 0.7 | 0.001813 |
| 11054 | hsa-miR-320 | Up in CIS | 0.5 | 0.005158 |
| 11006 | hsa-miR-205 | Down in CIS | −1.5 | 0.000144 |
| 11097 | hsa-miR-382 | Down in CIS | −0.5 | 0.000184 |
| 13127 | hsa-miR-200a* | Down in CIS | −0.5 | 0.000352 |
| 13183 | hsa-miR-487a | Down in CIS | −0.6 | 0.000712 |
| 13126 | hsa-miR-191* | Down in CIS | −0.7 | 0.000814 |
| 11041 | hsa-miR-29c | Down in CIS | −0.9 | 0.001999 |
| 11010 | hsa-miR-210 | Down in CIS | −0.4 | 0.003239 |
| 11043 | hsa-miR-302b | Down in CIS | −0.4 | 0.003416 |
| 11057 | hsa-miR-324-5p | Down in CIS | −0.7 | 0.00504 |
| 10931 | hsa-miR-127 | Down in CIS | −0.4 | 0.005705 |
| 11052 | hsa-miR-31 | Down in CIS | −0.5 | 0.007851 |
| 11070 | hsa-miR-345 | Down in CIS | −0.6 | 0.007908 |
| 10314 | hsa-miR-202* | Down in CIS | −1.0 | 0.015281 |

**LNA probe library 208010V7.1 from Exiqon, Denmark
^{a}Fold change values are log₂ transformed and generated from the median expression of the miRNAs in the groups compared.
^{b}Student's t-test p-values.
^{c}Only the ten most significantly up-and down-regulated miRNA are listed.

Approximate equal numbers of up- and down-regulated miRNAs were observed in comparing normal and cancer cells. These results contrasted with previous reports that showed an overall down-regulation of miRNAs in cancer (Lu et al. (2005) Nature 435: 834-838).

### Example 2

### Select miRNAs Predicted Subsequent Disease Progression to T2-4

In order to identify miRNAs for predicting subsequent disease progression to a muscle invasive stage, non-progressing tumors (18 Ta and 20 T1 tumors) were compared to progressing tumors (9 Ta and 20 T1 tumors). The most significantly regulated miRNA were identified using SAM (500 permutations, average 0% FDR as cut-off). 15 miRNAs were identified as significantly differentially expressed between the groups (refer to FIG. 1B). When only T1 tumors were considered in the analysis, it was found that a subset of the miRNAs listed in FIG. 1B were significantly differentially expressed, namely; miR-483, miR-526c, miR-503, miR-129, miR320, miR-145 and miR-133b.

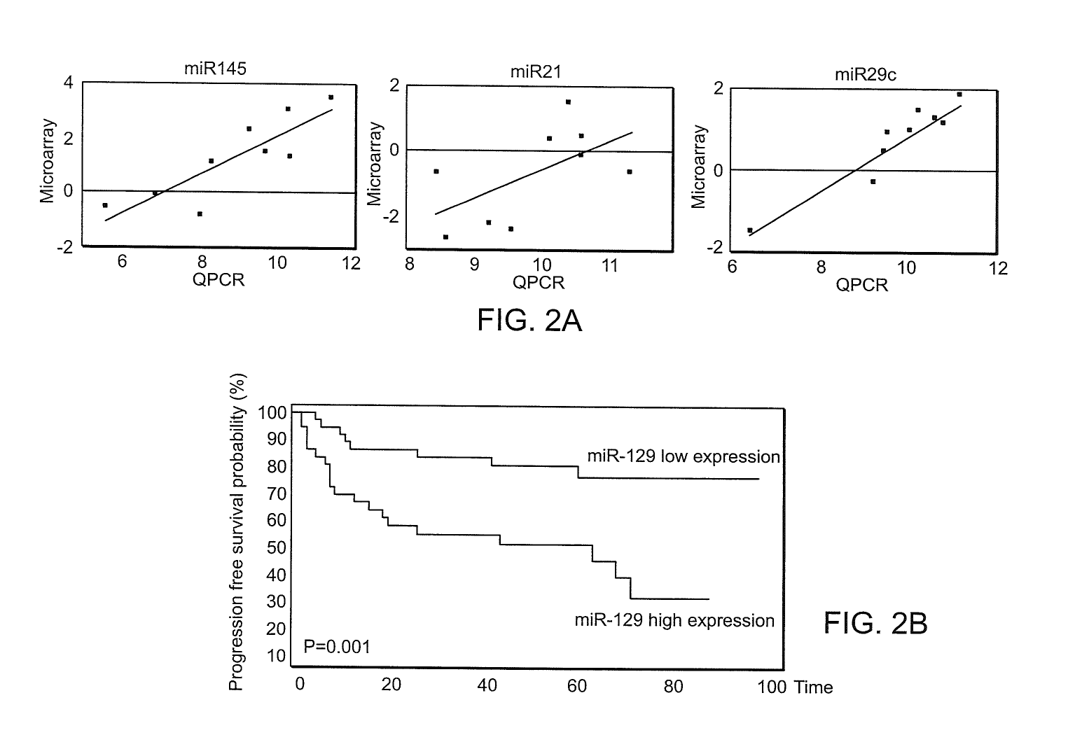

To identify the optimal set of miRNA for predicting disease outcome, a molecular maximum likelihood classifier was constructed using the leave-one-out cross-validation approach as previously described. The lowest error rate for cross-validation (25% error rate; chi2 test, p<0.0001) was obtained when both Ta and T1 tumors (N=67) were assessed using only 2 miRNAs in each of the cross-validation tests. Indeed, miR-129 and miR-133b were included in more than 75% of the tests. When focusing only on the T1 tumors (N=40) the lowest error rate was obtained using only 4 miRNAs (23% error rate; chi2 test, p<0.0005) in the cross-validation tests. Here, miR-129, miR-133b and miR-320 were included in more than 75% of the cross validations. The probability of progression-free survival as a function of miR-129 expression was plotted and is shown in FIG. 2B. Tumors with high expression of miR-129 exhibited a significant difference in progression-free survival as compared to tumors with low expression (log-rank test, P=0.001). When stratifying for disease stage in multivariate Cox regression analysis, it was observed that a hazard ratio of 5.1 (95% confidence interval, 2.2-12.2; P<0.001) could be attributed to elevated miR-129 expression. The most significantly regulated miRNAs between non-progressing and progressing tumors were determined and are listed above in Table 3.

### Example 3

### miRNAs Associated with Concomitant CIS

CIS (carcinoma-in-situ) has been identified as highly associated with subsequent disease progression. Several of the patients included in the present study were diagnosed with CIS in selected site biopsies at the present visit or at later cystoscopy examinations. SAM was again employed to identify miRNAs differentially expressed between tumors with or without CIS in selected site biopsies. When including both Ta and T1 tumors (N=63) in the analysis, 20 miRNAs were shown to exhibit significant differential expression between the two groups of tumors (refer to FIG. 1C). When only T1 tumors were included in the analysis (N=36), four differentially expressed miRNA (miR-129, miR-146, miR-17-5p, and miR-503) were identified. Consequently, miR-129 and miR503 were identified in both analyses. The most significantly differentially regulated miRNAs between non-CIS tumors and tumors with concomitant CIS were identified and are listed in Table 3 above.

As above, in selecting the optimal number of miRNAs for molecular classification of CIS, classifiers were constructed using expression data for all Ta and T1 tumors, or using expression data for T1 tumors only. When expression data from all tumors with known CIS status (N=63) were analyzed, the lowest classifier error rate was observed when 10 miRNAs were included in cross-validations (14 errors; chi-test, p<0.00006). Among these 10 miRNAs, miR-129, miR-191*, miR-200a*, miR-205, miR-382, miR-487a, miR-498 and miR-503 were included in more than 75% of cross-validation tests. When only T1 tumors (N=36) were analyzed, the lowest classifier error rate was observed when using 7 miRNAs for cross-validation (11 errors; chi test, p=0.02). Among these 7 miRNAs, miR-146a, miR-17-5p and miR-210 were observed in more than 75% of the tests. Consequently, no overlap between these classifiers (all tumors vs. T1 only) was found.

### Example 4

### miRNAs Predicted Post-Chemotherapy Survival for Patients with Advanced Disease

The prospect of identifying miRNAs that predicted survival following chemotherapy in patients with advanced disease was assessed. A SAM survival analysis performed upon the expression dataset with 500 permutations and an average FRD of 0% identified miR-29c as significantly correlated with the survival of patients with T2-4 tumors. Specifically, down-regulation of miR-29c was correlated with short survival time. Interestingly, miR-29c was also found to be significantly down-regulated in CIS tumors and in progressing non-muscle invasive tumors. miR-29 was previously demonstrated as involved in regulating apoptosis (Mott et al. (2007) Oncogene, advance online publication: doi:10.1038/sj.one.1210436) and down-regulation of miR-29 was previously shown to correlate with poor prognosis in chronic lymphocytic leukaemia (CLL; Pekarsky et al. (2006) Cancer Res 66: 11590-11593).

### Example 5

### Comparison Between Array miRNA Measurements and QPCR Measurements of Mature miRNA

The microarray LNA probes hybridizeD to pre-, pri-, and mature forms of the miRNAs. Therefore, the relationship between expression of the mature form of selected miRNAs measured by QPCR assays and the expression data obtained from the microarray platform was investigated. QPCR (ragman) assays were used to measure the expression of 11 of the identified miRNAs (miR-145, miR-21, miR-126*, miR-200c, miR-193a, miR-130b, miR-199a, miR-141, miR29a, miR29c, and miR129) in samples also analyzed by microarray expression profiling. The assays showed an average Pearson correlation between QPCR assays and microarray data of 0.8 (range 0.6-0.9), with miR-129 omitted from this consideration for failure to amplify. The comparisons for miR-145, miR-21 and miR-29c were performed and results are shown in FIG. 2A. Non-normalized QPCR data showed larger similarities to microarray data than when performing normalization using the normalization probes described in materials and methods. In fact, the commonly used normalizer RNU6B showed considerable variation. Consequently, no normalization of the QPCR data was performed in the present study.

### Example 6

### Detection of miRNA Expression by In Situ Hybridization

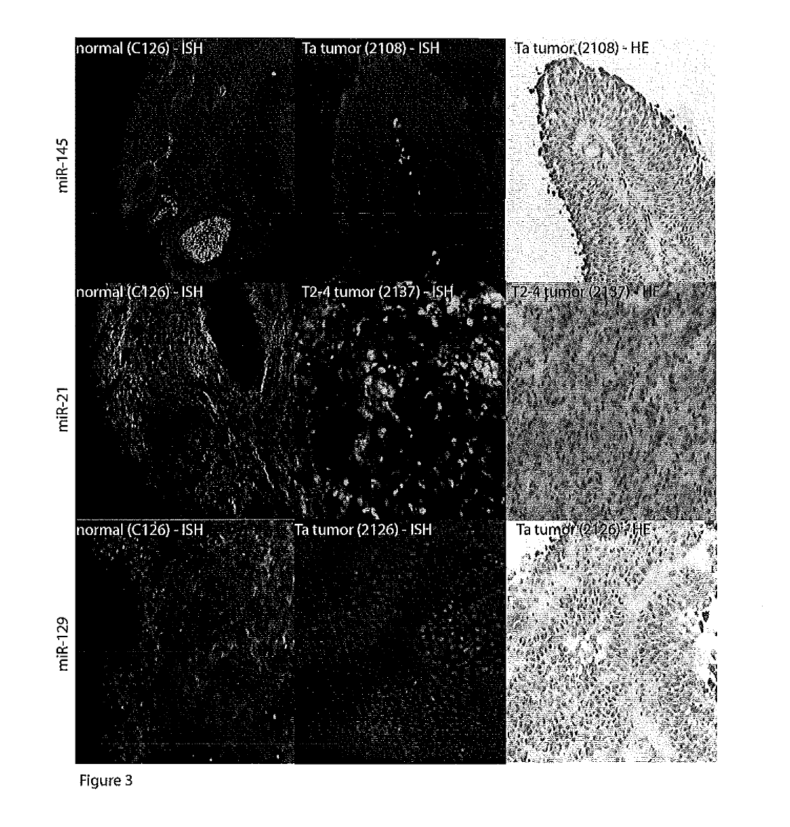

LNA based FITC labeled probes were used for detection of miR-145, miR-21 and miR-129 expression (refer to FIG. 3). miR-145 expression was identified in connective tissue and stroma cells, whereas no expression of miR-145 was detected in urothelial or urothelial carcinoma cells. miR-21 expression was not detected in normal tissue biopsies; however, in tumor tissue, miR-21 expression was identified within cancer cells. miR-129 expression was detected both in normal urothelial cells and in carcinoma cells in tumor tissue. Consequently, the expression of miR-129 in carcinoma cells makes this miRNA a strong candidate for further functional studies in cell line studies. Localization of miRNA expression is very important, as results from microarray studies of crude tumor biopsies could in some cases reflect differences in tissue compositions rather than true differences in miRNA expression within specific types of cells.

### Example 7

### Measurement of miR-129 Expression Levels

Median expression levels of informative miRNA miR-129 were used to determine a cut-off level for classification of bladder cancer samples. In the preceding Examples, median expression of miR-129 was −1.04 (control vs. tumor samples, with values corresponding to log₂ measurements). Samples with higher values were classified as progressing samples and samples with lower values were classified as non-progressing samples. This cut-off value yielded a sensitivity of 71% and a specificity of 66%. Adjustment of the cut-off value to −1.16 yielded a sensitivity of 86% and a specificity of 61%.

### Example 8

### Classification Using Single miRNA Expression Values

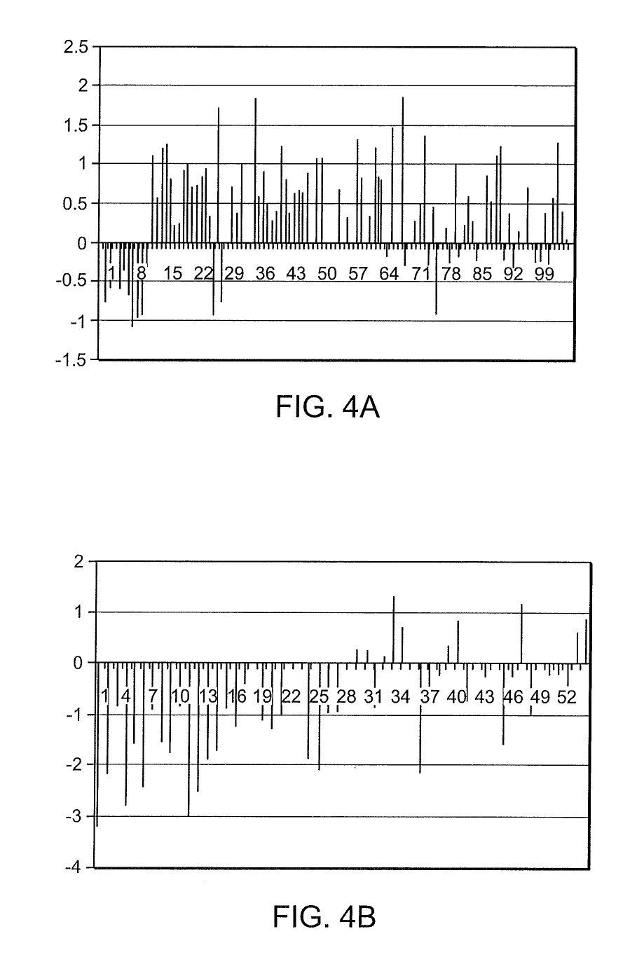

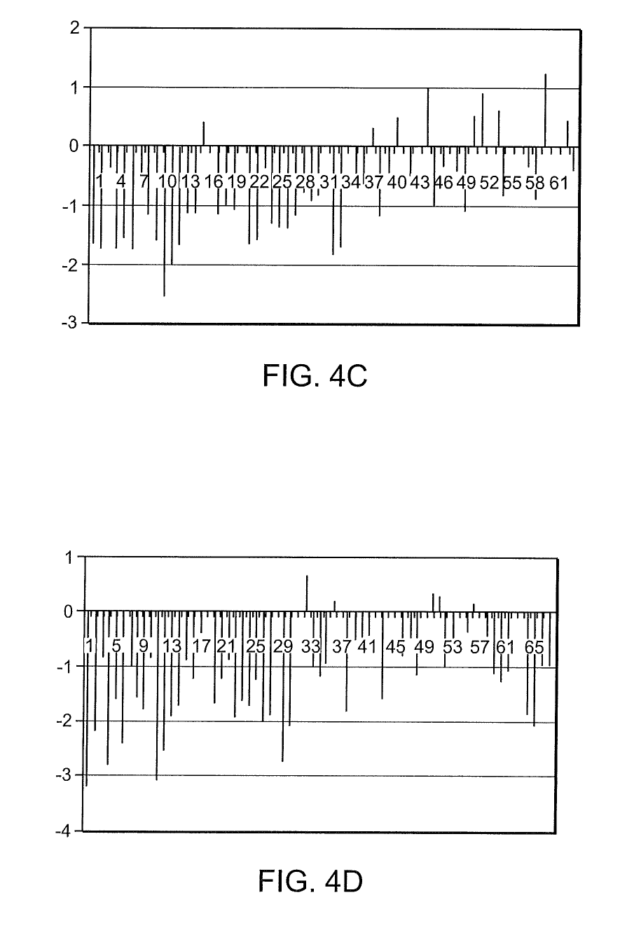

Cutoff values for use in classification of samples via measurement of single miRNA expression levels were selected and were associated with measurable levels of both sensitivity and specificity (critical to assessing predictive value of such miRNA expression levels, not only for single miRNAs, but also for miRNA signatures). Calculations of cutoff values, specificity values and sensitivity values were performed for normal vs cancer biopsies using miR-193a (FIG. 4A); Ta vs T2 bladder cancer using miR-129 (FIG. 4B); no CIS vs CIS using miR-503 (FIG. 4C); and no progression vs progression using miR-129 (FIG. 4D).

miR-193a was assessed in normal vs. tumor samples and showed a median level in normal samples of −0.6, with SD (Standard Deviation) in normal samples of 0.34. A cutoff value corresponding to (median+SD) was set at 0.08. For this value, sensitivity was observed to be 83%, with 82% specificity, for predictive use of measured elevation of miR-193a to identify a bladder cancer tumor in a sample from a subject.

miR-129 was assessed in Ta vs. T2 bladder cancer samples and showed a median level in Ta samples of −1.6, with SD in Ta samples of 0.76. A cutoff value corresponding to (median+SD) was set at −0.86. For this value, sensitivity was observed to be 81%, with 85% specificity, for predictive use of measured miR-129 elevation to identify T2-T4 stage bladder cancer in a sample from a subject.

miR-503 was assessed in no CIS vs CIS samples and showed a median level in no CIS samples of −1.1, with SD in no CIS samples of 0.75. A cutoff value corresponding to (median+SD) was set at −0.38. For this value, sensitivity was observed to be 69%, with 72% specificity, for predictive use of measured miR-503 elevation to identify CIS in a sample from a subject.

miR-129 was also assessed in no progression vs progression samples and showed a median level in no progression samples of −1.65, with SD in no progression samples of 0.87. A cutoff value corresponding to (median+SD) was set at −0.78. For this value, sensitivity was observed to be 52%, with 82% specificity, for predictive use of measured miR-129 elevation to identify progressive bladder cancer in a sample from a subject.

### Example 9

### Further Analysis of Expanded Data Set

Further analysis of the above data was performed within an expanded study that involved profiling the expression of 290 unique human miRNAs in 106 bladder cancers and 11 normal samples using spotted LNA-based oligonucleotide microarrays. The expression of these 290 unique human miRNA genes in cell lines (HU609, T24, SW780, RT4, and J82) and in immortalized normal urothelim samples (HCV29 and HT1376) was profiled. Expression of these 290 unique human miRNAs was assessed in 11 biopsies of normal urothelium obtained from healthy individuals, in 30 Ta tumors (11 progressed to T2-4), in 49 T1 tumors (26 progressed to T2-4) and in 27 T2-4 tumors. Patients with non-progressing Ta and T1 tumors had a median follow-up time of 64 months and patients with progressing tumors had a median time to progression of 10 months. The reproducibility of the microarray platform was tested using cell lines. Technical replicate measurements of cell lines HU609 and T24 showed that the miRNA oligonucleotide microarray platform gave highly reproducible data with an average Pearson correlation of 0.90 (data no shown).

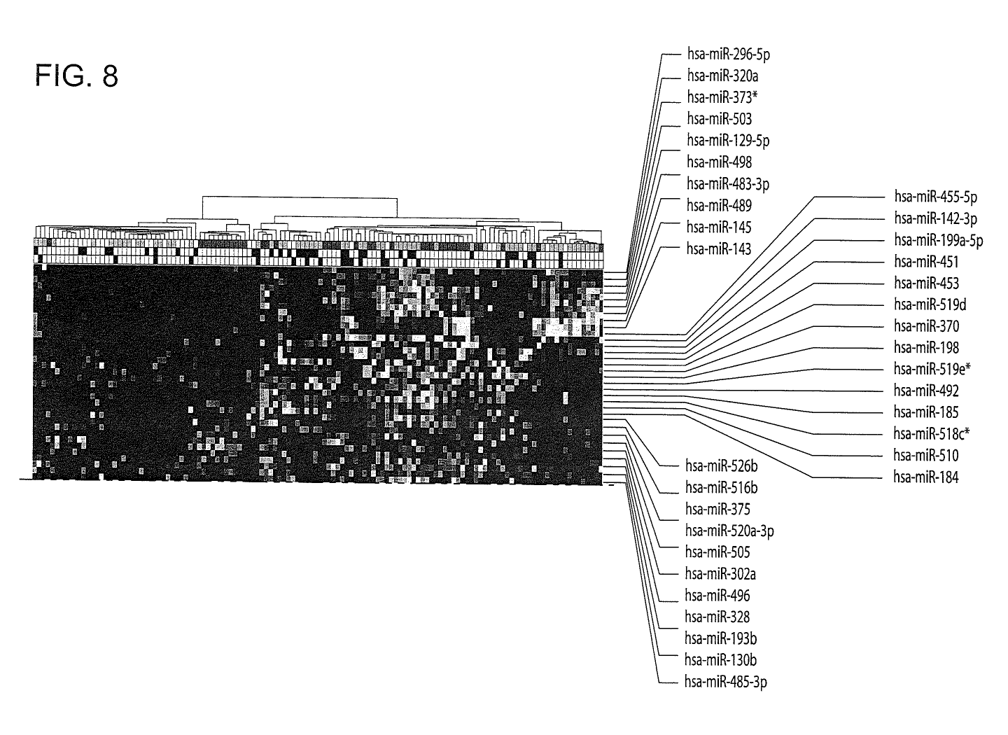

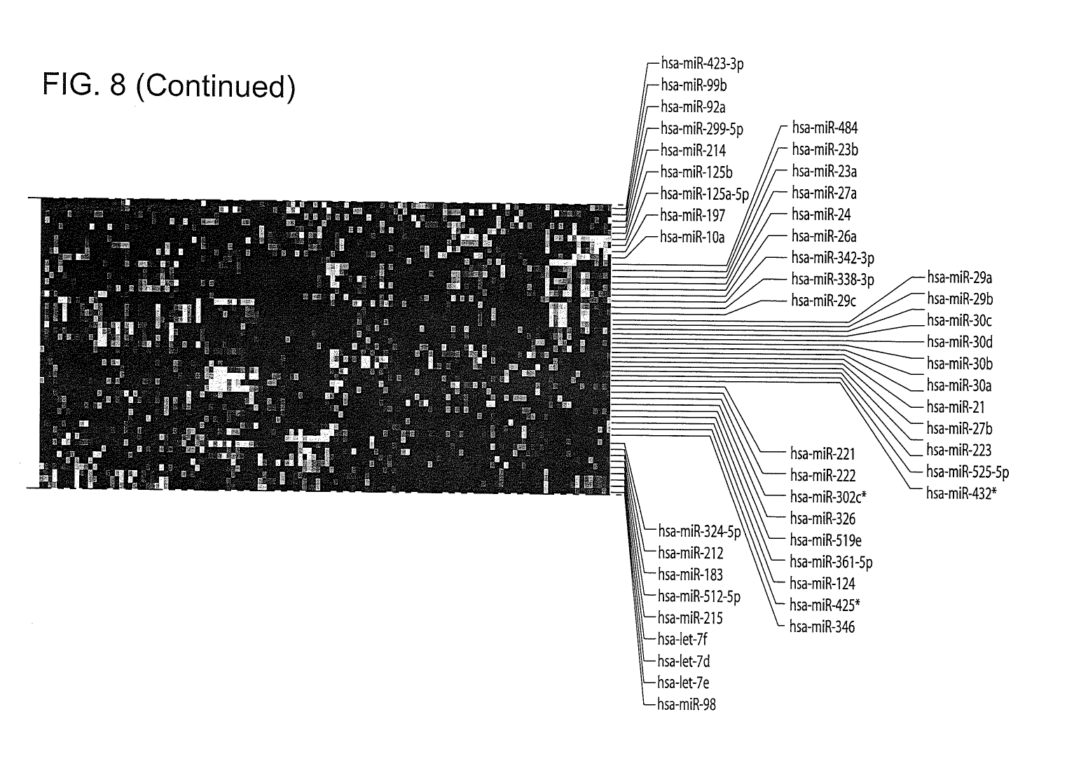

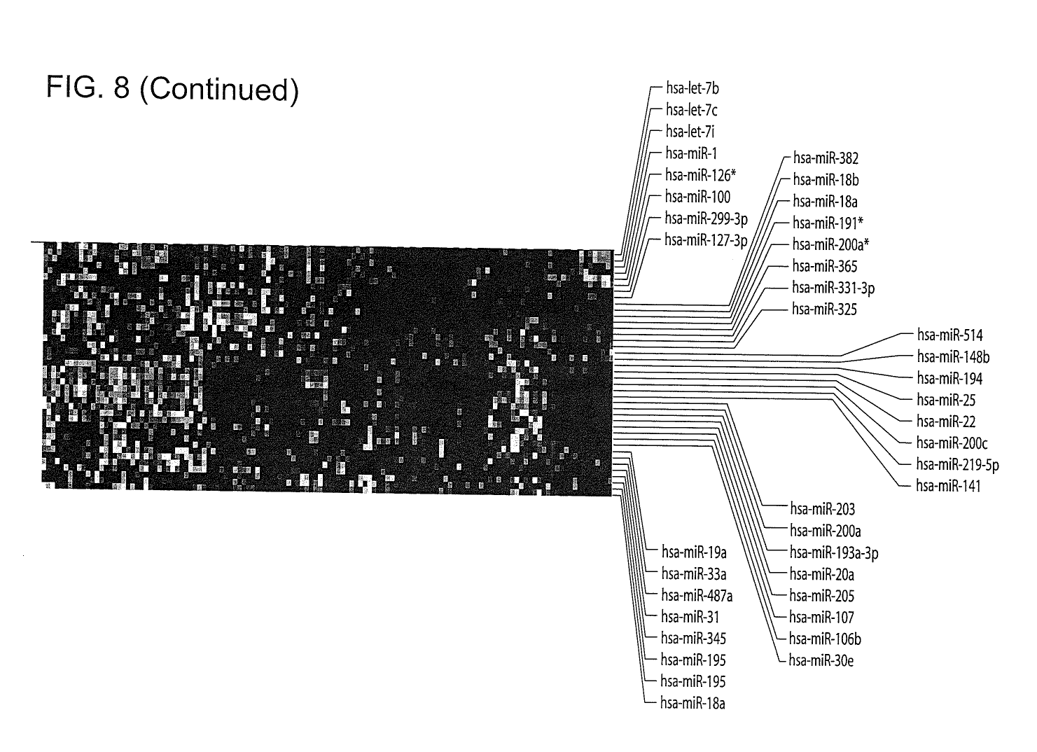

Hierarchical cluster analysis of the 122 miRNAs with expression above background in a minimum of 25% of samples analyzed separated the samples into one arm (see FIG. 8—left side cluster arm) with an overrepresentation of Ta tumors and T1 tumors without subsequent progression, and another arm (right side aim) with and overrepresentation of T1 tumors with subsequent progression (p=0.002, chi²-test) clustering among the T2-4 tumors. Overall, the expression of miRNA in the cell lines and the immortalized normal urothelial cells resembled the expression of miRNA in Ta tumors. Furthermore, the cluster analysis showed very little variation in miRNA expression between the different cell lines and between immortalized normal urothelial cells. The normal urothelial biopsies clustered together with the invasive samples—probably due to tissue composition.

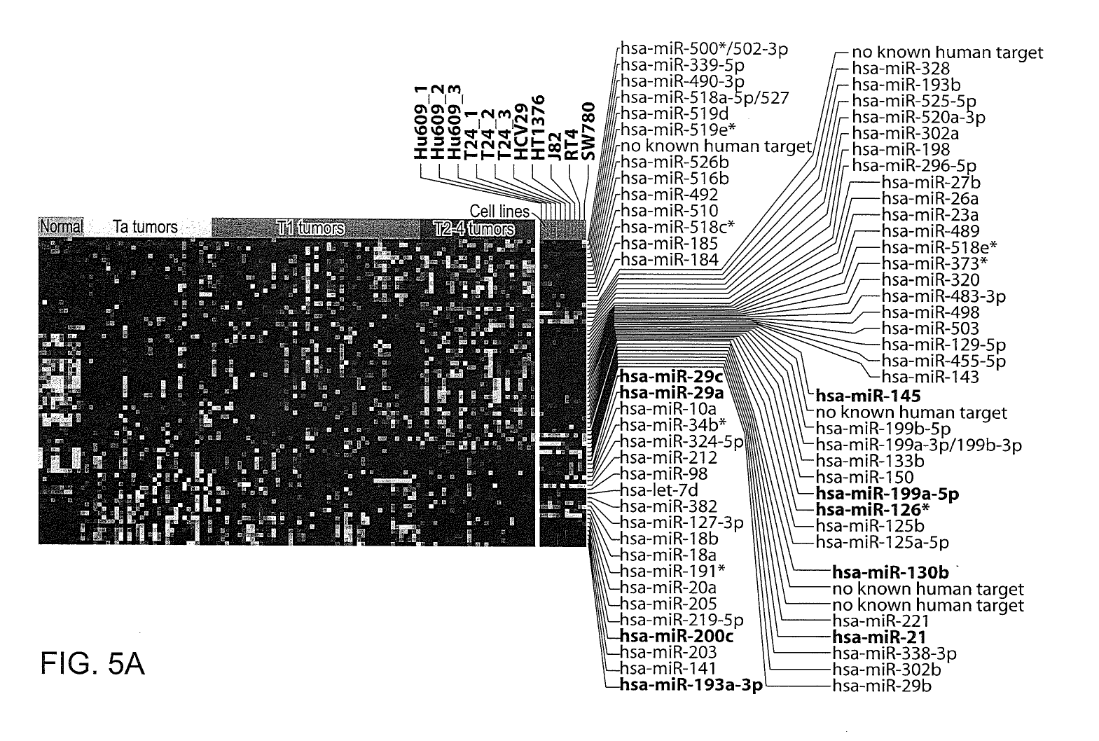

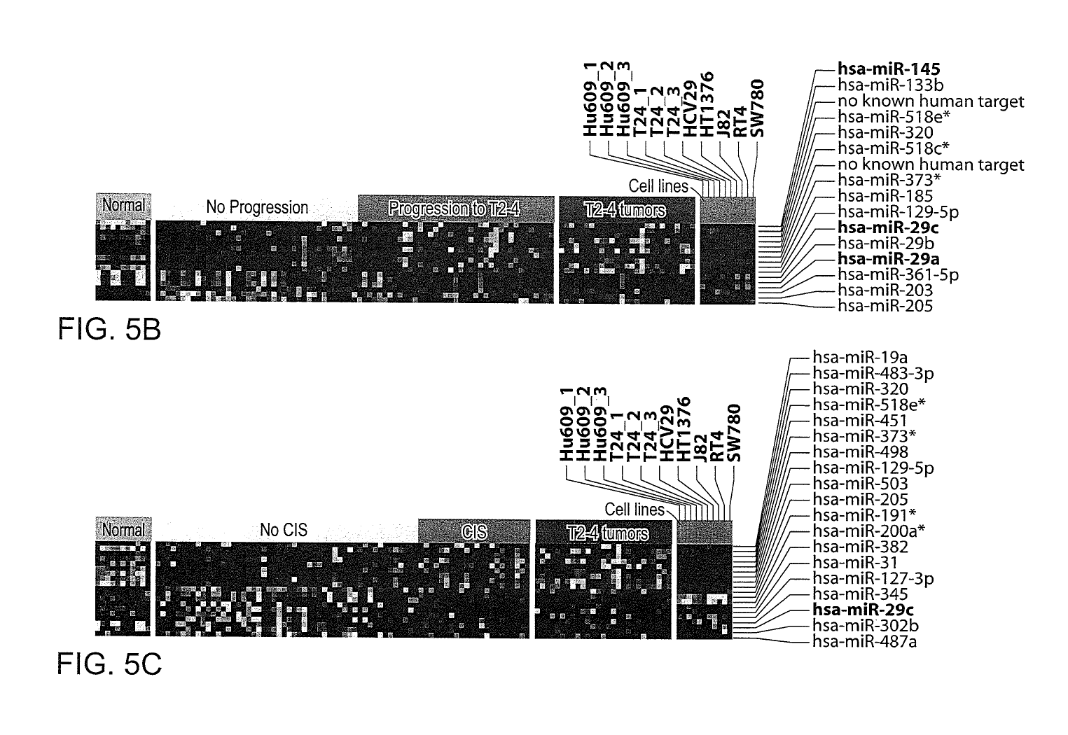

miRNAs differentially expressed between normal and cancer and between different stages of cancer were then identified. Specifically, miRNAs differentially expressed between normal urothelium and tumor samples were assessed, and between Ta, T1 and T2-4 stages using SAM (Significance Analysis of Microarrays). One thousand permutations were performed, and an average false discovery rate (FDR) of 0% was used as the cut-off level for selecting differentially expressed miRNAs. The 80 probes against human miRNAs that showed significant differential expression are shown in FIG. 5A. Several miRNAs were found to be specifically associated with normal tissue, Ta tumors and T2-4 tumors; however, the T1 tumors showed expression similarities to either Ta or T2-4 tumors. The most significantly regulated miRNAs were identified and listed in Table 4.

TABLE 4
microRNAs differentially expressed between normal tissue and
bladder cancer and between Ta and T2-4 tumors. Only the most
significant are listed (p < 0.0001; students t-test).

| | | | | | |
|---|---|---|---|---|---|
| Probe | | Fold | | Detected | Chromosome |
| ID^{a} | miRNA target | change^{b} | t-test^{c} | (%)^{d} | location |

miRNAs upregulated in cancer

| | | | | | |
|---|---|---|---|---|---|
| 403 | miR-519e* | 0.8 | 4.1E−08 | 93 | 19q13.41 |
| 559 | miR-193a-3p | 1.0 | 2.8E−07 | 61 | 17q11.2 |
| 459 | miR-21 | 1.9 | 2.9E−07 | 87 | 17q23.3 |
| 581 | miR-20a | 1.1 | 7.8E−06 | 70 | 13q31.3 |
| 119 | miR-198 | 1.0 | 2.5E−05 | 95 | 3q13.33 |
| 711 | miR-510 | 1.4 | 2.7E−05 | 88 | Xq27.3 |
| 551 | miR-184 | 0.9 | 7.5E−05 | 75 | 15q25.1 |
| 693 | miR-492 | 1.4 | 9.4E−05 | 97 | 12q22 |

miRNAs downregulated in cancer

| | | | | | |
|---|---|---|---|---|---|
| 429 | miR-455-5p | −1.4 | 4.4E−11 | 45 | 9q32 |
| 427 | miR-143 | −1.4 | 6.9E−09 | 74 | 5q33.1 |
| 525 | miR-145 | −2.8 | 2.1E−08 | 93 | 5q33.1 |
| 505 | miR-126* | −1.0 | 1.8E−07 | 43 | 9q34.3 |
| 603 | miR-26a | −0.7 | 4.4E−06 | 73 | 3p22.2/12q14.1 |
| 57 | miR-125b | −1.2 | 7.2E−06 | 91 | 11q24.1/21q21.1 |
| 699 | miR-498 | −0.9 | 2.1E−05 | 90 | 19q13.41 |
| 243 | miR-489 | −0.9 | 2.8E−05 | 41 | 7q21.3 |
| 257 | miR-503 | −1.2 | 4.1E−05 | 99 | Xq26.3 |
| 163 | miR-29a | −0.8 | 4.3E−05 | 79 | 7q32.3 |
| 167 | miR-302b | −0.4 | 4.6E−05 | 20 | 4q25 |
| 165 | miR-29c | −1.0 | 5.8E−05 | 96 | 1q32.2 |

miRNAs upegulated in T2-4 compared to Ta

| | | | | | |
|---|---|---|---|---|---|
| 257 | miR-503 | 1.3 | 1.1E−08 | 99 | Xq263 |
| 509 | miR-129-5p | 1.4 | 2.3E−08 | 97 | 7q31/11p11.2 |
| 625 | miR-320a | 0.9 | 7.3E−08 | 95 | 8p21.3 |
| 113 | miR-193b | 0.9 | 2.8E−07 | 37 | 16p13.12 |
| 877 | miR-483-3p | 1.2 | 5.2E−07 | 77 | 11p15.5 |
| 699 | miR-498 | 0.7 | 1.2E−06 | 90 | 19q13.41 |
| 273 | miR-516b | 0.6 | 1.5E−06 | 43 | 19q13.41 |
| 631 | miR-328 | 0.6 | 3.0E−06 | 41 | 16q22.1 |
| 657 | miR-373* | 0.9 | 1.5E−05 | 91 | 19q13.41 |
| 403 | miR-519e* | 0.5 | 1.5E−05 | 93 | 19q13.41 |
| 745 | miR-526b | 0.9 | 2.0E−05 | 48 | 19q13.41 |
| 607 | miR-296-5p | 0.6 | 2.6E−05 | 99 | 20q13.32 |
| 691 | miR-490-3p | 0.4 | 3.5E−05 | 24 | 7q33 |
| 243 | miR-489 | 0.4 | 1.0E−04 | 41 | 7q21.3 |

miRNAs downregulated in T2-4 compared to Ta

| | | | | | |
|---|---|---|---|---|---|
| 857 | miR-18b | −0.6 | 1.3E−07 | 79 | Xq26.2 |
| 145 | miR-219-5p | −1.1 | 4.7E−06 | 68 | 6q21.32/9q34.11 |
| 751 | miR-98 | −0.8 | 5.4E−06 | 85 | Xp11.22 |
| 521 | miR-141 | −1.5 | 1.0E−05 | 78 | 12p13.31 |
| 575 | miR-200c | −1.0 | 4.3E−05 | 76 | 12p13.31 |
| 397 | miR-191* | −0.8 | 5.0E−05 | 91 | 3q21.31 |
| 577 | miR-203 | −1.2 | 5.4E−05 | 72 | 14q32.33 |
| 197 | miR-34b* | −0.3 | 8.7E−05 | 9 | 11q23.1 |

^{a}Probe ID from LNA library 208010V7.1 (Exiqon, Denmark).
^{b}Fold change values are log2 transformed and generated from the median expression of the miRNAs in the groups compared.
^{c}Students t-test p-values.
^{d}Percent of samples in which the miRNA is detected above the background filter.

miRNAs differentially expressed between non-progressing and progressing non-muscle invasive tumors were also identified. Prediction of subsequent disease progression for patients with non-muscle invasive tumors has been a difficult and error-prone process, and to date, no molecular markers for predicting progression have been implemented in clinical routine. In order to identify miRNAs for predicting subsequent disease progression to a muscle invasive stage, non-progressing tumors were compared to progressing tumors, and the most significantly regulated miRNAs were identified using SAM (1000 permutations, average 5% FDR as cut-off was used). Fourteen miRNAs were found to be significantly differentially expressed between the groups (FIG. 5B). The most significantly regulated miRNAs between non-progressing and progressing tumors were identified and listed in Table 5.

TABLE 5
microRNAs differentially expressed between non-progressing and
progressing and between tumors with and without concomitant CIS.

| | | | | | |
|---|---|---|---|---|---|
| Probe | | Fold | | Detected | Chromosome |
| ID^{a} | miRNA target | change^{b} | t-test^{c} | (%)^{d} | location |

miRNAs upregulated in progressing tumors

| | | | | | |
|---|---|---|---|---|---|
| 509 | miR-129-5p | 0.7 | 0.001 | 97 | 7q31/11p11.2 |
| 849 | miR-518c* | 0.3 | 0.002 | 94 | 19q13.41 |
| 457 | miR-185 | 0.4 | 0.003 | 98 | 22q11 |
| 67 | miR-133b | 0.5 | 0.003 | 19 | 6p12.2 |
| 657 | miR-373* | 0.3 | 0.005 | 91 | 19q13.41 |
| 625 | miR-320 | 0.5 | 0.008 | 95 | 8p21.3 |
| 525 | miR-145 | 0.5 | 0.022 | 93 | 5q33.1 |

miRNAs downregulated in progressing tumors

| | | | | | |
|---|---|---|---|---|---|
| 165 | miR-29c | −0.6 | 0.001 | 96 | 1q32.2 |
| 611 | miR-29b | −0.8 | 0.001 | 89 | 1q32.2/7q32.3 |
| 163 | miR-29a | −0.5 | 0.003 | 79 | 7q32.3 |
| 199 | miR-361-5p | −0.3 | 0.005 | 42 | Xq21.2 |
| 577 | miR-203 | −0.8 | 0.004 | 72 | 14q32.33 |
| 579 | miR-205 | −1.4 | 0.014 | 56 | 1q32.2 |

miRNAs upregulated in tumors with concomitant CIS

| | | | | | |
|---|---|---|---|---|---|
| 257 | miR-503 | 0.9 | <0.001 | 99 | Xq26.3 |
| 699 | miR-498 | 0.6 | 0.001 | 90 | 19q13.41 |
| 509 | miR-129-5p | 0.9 | 0.001 | 97 | 7q31/11p11.2 |
| 657 | miR-373* | 0.6 | 0.002 | 91 | 19q13.41 |
| 877 | miR-483-3p | 0.6 | 0.009 | 77 | 11p15.5 |
| 625 | miR-320 | 0.3 | 0.014 | 95 | 8p21.3 |
| 365 | miR-451 | 0.6 | 0.015 | 87 | 17q11.2 |
| 123 | miR-19a | 0.1 | 0.050 | 28 | 13q31.3 |

miRNAs downregulated in tumors with concomitant CIS

| | | | | | |
|---|---|---|---|---|---|
| 579 | miR-205 | −1.4 | <0.001 | 56 | 1q32.2 |
| 397 | miR-191* | −0.6 | 0.001 | 91 | 3p21.31 |
| 667 | miR-382 | −0.5 | 0.001 | 61 | 14q32.31 |
| 59 | miR-127-3p | −0.7 | 0.001 | 97 | 14q32.31 |
| 165 | miR-29c | −0.8 | 0.001 | 96 | 1q32.2 |
| 845 | miR-200a* | −0.5 | 0.002 | 61 | 1p36.33 |
| 623 | miR-31 | −0.4 | 0.002 | 30 | 9p21.3 |
| 167 | miR-302b | −0.3 | 0.003 | 20 | 4q25 |
| 641 | miR-345 | −0.5 | 0.004 | 26 | 14q32.2 |
| 433 | miR-487a | −0.6 | 0.006 | 34 | 14q32.31 |

miRNAs downregulated in FGFR3 mut tumors

| | | | | | |
|---|---|---|---|---|---|
| 699 | miR-498 | −0.7 | 0.005 | 90 | 19q13.41 |
| 509 | miR-129-5p | −1.4 | 0.006 | 97 | 7q3/11p11.2 |
| 525 | miR-145 | −1.1 | 0.009 | 93 | 5q33.1 |

miRNAs upregulated in FGFR3 mut tumors

| | | | | | |
|---|---|---|---|---|---|
| 83 | miR-148b | 0.8 | 0.005 | 46 | 12q13.13 |
| 53 | miR-10b | 0.8 | 0.007 | 15 | 2q31.1 |
| 181 | miR-324-5p | 1.7 | 0.016 | 97 | 17q13.1 |

^{a}Probe ID from LNA library 208010V7.1 (Exiqon, Denmark).
^{b}Fold change values are log2 transformed and generated from the median expression of the miRNAs in the groups compared.
^{c}Students t-test p-values.
^{d}Percent of samples in which the miRNA is detected above the background filter.

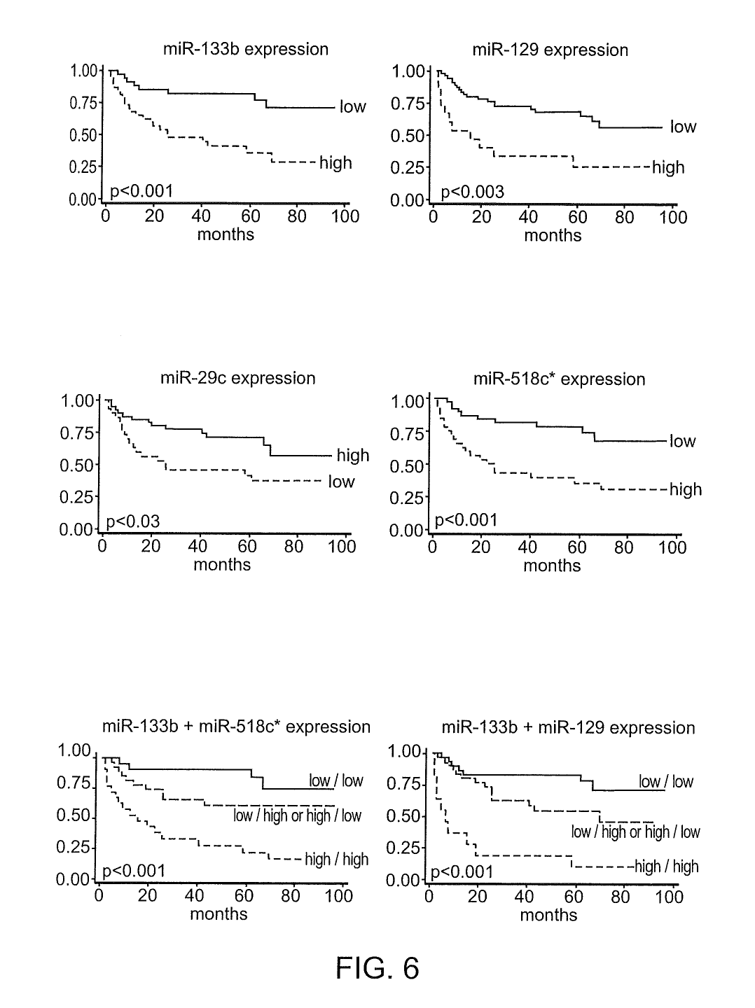

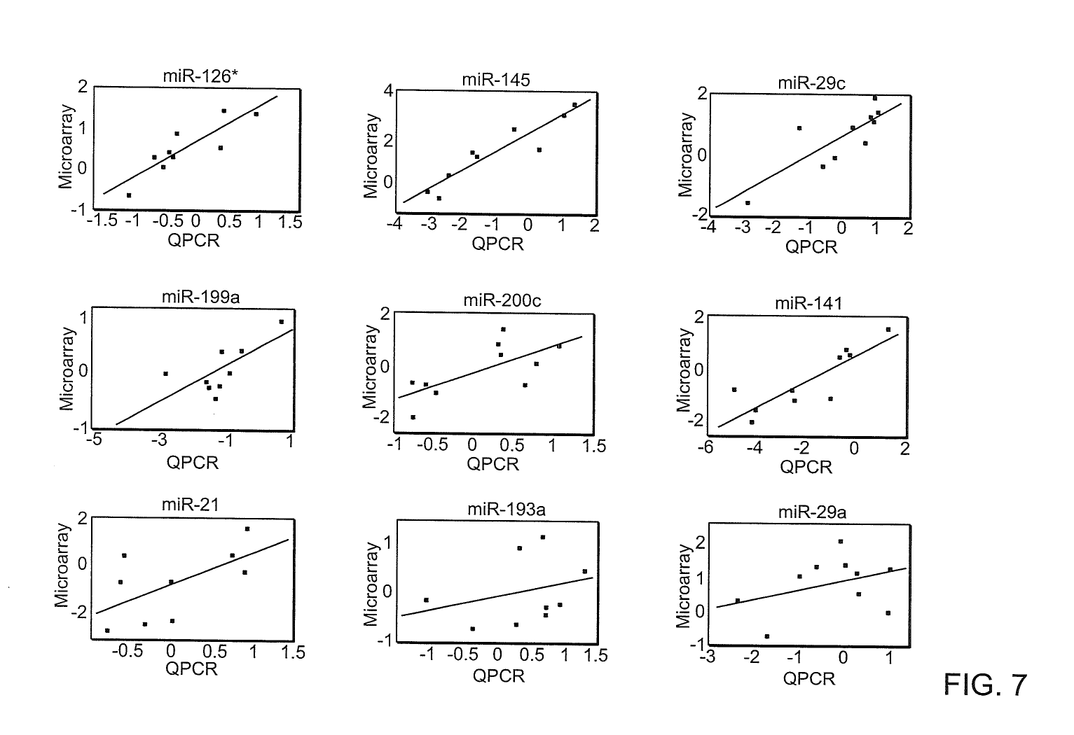

Kaplan-Meier graphs of some the most significantly regulated miRNAs (i.e., miR-129, miR-133b, miR-518c* and miR-29c) were constructed (see FIG. 6). Optimal cut-off limits were determined for these miRNAs using ROC-curves. Mutivariate Cox regression analysis showed that all 4 miRNAs were significantly associated with disease progression (miR-133b: HR 3.5, p=0.002; miR-518c*: HR 3.2, p=0.003; miR-129: HR 3.0, p<0.02; miR-29c: HR 2.1, p<0.05) when correcting for disease stage and grade. Combinations using miR-133b+miR-518c* and miR-133b+miR-129 showed an even better prediction of outcome (FIG. 6, bottom panels). Multivariate Cox regression analysis of the combined miR-129 and miR133b measure resulted in a hazard ratio of 9.2 (p<0.001) when comparing the mir-129+/miR133b+group to the miR-129-/miR133b-group. For the miR-129/miR-518c* combination, the multivariate Cox regression analysis showed a hazard ratio of 7.3 (p<0.001).

miRNAs differentially expressed between non-muscle invasive tumors with and without concomitant CIS were also identified. Several patients included in this study were diagnosed with CIS in selected site biopsies at the present visit or at later cystoscopy examinations (for examples, see Table 1). SAM was again used to identify miRNAs differentially expressed in tumors with or without CIS in selected site biopsies. When including both Ta and T1 tumors (N=63) in the analysis, 19 miRNAs were identified with significant differential expression between the two groups of tumors (FIG. 5C). The most significantly regulated miRNAs between no CIS and tumors with concomitant CIS were presented above in Table 5. Optimal cut-off limits were determined for the most significant miRNAs using ROC-curves. Multivariate logistic regression analysis showed that the miRNA expression levels were significantly associated with CIS status (miR-503: OR 5.6, p=0.004; miR-205: OR 3.8, p<0.04; miR-382: OR 3.2, p=0.02; miR-191: OR 2.2, p<0.04) when correcting for disease stage and grade. Again, comparing the miR-503+/miR-205+ group to the miR-503-/miR-205-group by multivariate logistic regression analysis gave an odds ratio of 10.6 (p=0.006) when adjusting for stage and grade. Other combinations gave similar high odds ratios (results not shown).

miRNAs differentially expressed between tumors with mutated and wild type FGFR3 were also identified. In 19 cases, information about FGFR3 mutation status was known from a previous study (Zieger et al. Clin Cancer Res 2005; 1 1(21):7709-19). When comparing FGFR3 wild type tumors to FGFR3 mutated tumors using SAM analysis (1000 permutations, 5% FDR) identified six miRNAs were identified that showed statistically significant differential expression (Table 5).

Real-time RT-PCR was used to validate miRNA expression. The microarray LNA probes hybridized to both pre-, pri-, and mature forms of the miRNAs. Therefore, the relationship between expression of the mature form of selected miRNAs measured by real-time RT-PCR assays and the expression data obtained from the microarray platform was investigated. RT-PCR (Taqman) assays were used to measure the expression of 10 miRNAs (i.e., miR-145, miR-21, miR-126*, miR-200c, miR-193a, miR-199a, miR-141, miR-29a, miR-29c, and miR-129) in samples analyzed by microarray expression profiling. The assays showed an average Pearson correlation to the microarray data of 0.8 (range 0.6-0.9), except for miR-129 that failed to amplify. The comparisons between array data and real-time RT-PCR data were assessed and graphed (FIG. 7). An in silica approach was also applied to analyze changes in expression of mRNA target molecules of some of the identified miRNAs (data not shown).

### Example 10

### Using a Microarray Platform to Measure miRNA Expression and to Predict Cancer Outcome

A predictive set of miRNA for bladder cancer progression are identified and assessed in a sample using a two color LNA-based oligonucleotide microarray platform with a standard reference using fixed microarray scanner settings and fixed software parameters for calculating miRNA expression levels in each sample measured. miRNA expression levels are measured for predictive miRNAs (e.g., sets of informative miRNAs as described above) in approximately 50 training samples (25 progressing and 25 non-progressing) and a maximum likelihood classifier is built and trained as described in Dyrskjot et al. Nat. Genet. (2003) 33:90-6. In the training of the classifier, the optimal cut-off point is selected between the groups in order to increase the sensitivity. Using this approach, the classifier groups (progression and non-progression) are then predefined and new tumor samples are classified according to the predefined groups using this classifier. The classifier works using from one to several predictive miRNAs. For each analysis, a standard reference sample is analyzed for purpose of yielding consistent classification values in each run. Such measurements may be carried out in triplicate.

### Example 11

### Using a QPCR Platform to Measure miRNA Expression and to Predict Cancer Outcome

Non-array-based methods known in the art are also used to assess miRNA expression levels and/or patterns. For example, QPCR using standard ABI Taqman assays is used to assess miRNA expression levels and/or patterns. An ABI7900 settings are standardized and the maximum likelihood outcome classifier is then built using the same approach as described above for the microarray platform (50 training samples). Optimal cut-off points are generated for use on new tumor samples to increase sensitivity. As above, for each assay a standard reference sample is analyzed for purpose of yielding consistent classification values. The QPCR based classifier employs from 1 to several predictive miRNAs. Such measurements may be carried out in triplicate.

### Example 12

### Clinical Use of miRNA Arrays

An exemplary clinical implementation of miRNA arrays for measuring, e.g., the miRNA signatures reported herein, involves performance of the following steps:
1. Extraction of total RNA from biopsies obtained from tumors or suspected tumor lesions;
2. Labeling of (test sample) RNA, e.g., using HY3 labeling kit from Exiqon, DK;
3. Labeling of a standard reference pool of RNA using distinct labelling from test sample RNA, e.g., labeled using a HY5 labeling kit from Exiqon, DK;
4. Hybridization and scanning of the miRNA arrays described herein (control samples included for each end-point);
5. Data are normalized and relative intensity measures are obtained;
6. If control values are acceptable, each biopsy is classified according to predefined cut-off values using the multi-miRNA signatures described herein;
7. A classification value is generated for each biopsy (e.g., normal or tumor; Ta or T2; carcinoma-in-situ or no carcinoma-in-situ; progression or no progression);
8. The patient receives treatment according to combined histopathological, clinical and molecular classification data.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

TABLE 1
Clinical and Histopathological Parameters for Patients Included in this Study

| | | | | | | | | | Death cause | | |
|---|---|---|---|---|---|---|---|---|---|---|---|
| | | | | | | | | | (0 = alive, | | |
| | | | | | Progression | | | | 1 = died from | | |
| | | | | | (0 no | | | | bladder cancer, | | |
| | | | | | progression, | | Progression | | 2 = other cause, | | |
| | | | | CIS | 1 prog to T1, | | free | Follow-up | 3 unknown | | |
| Material | Sample # | Stage | Grade | diagnosis | 2 prog to T2+) | Follow-up* | survival | (total) | cause | SEX | AGE |
| Normal | \acute{ }9-99 | — | — | — | — | — | — | — | — | — | — |
| Normal | 316-99 | — | — | — | — | — | — | — | — | — | — |
| Normal | 335-99 | — | — | — | — | — | — | — | — | — | — |
| Normal | 337-99 | — | — | — | — | — | — | — | — | — | — |
| Normal | 340-99 | — | — | — | — | — | — | — | — | — | — |
| Normal | 341-99 | — | — | — | — | — | — | — | — | — | — |
| Normal | 343-99 | — | — | — | — | — | — | — | — | — | — |
| Normal | 344-99 | — | — | — | — | — | — | — | — | — | — |
| Normal | 345-99 | — | — | — | — | — | — | — | — | — | — |
| Normal | 55-99 | — | — | — | — | — | — | — | — | — | — |
| Normal | 70-99 | — | — | — | — | — | — | — | — | — | — |
| Tumor | 1058-2 | Ta | 2 | no cis | 2 | 42 | 42 | 43 | 1 | F | 67 |
| Tumor | 1093-1 | Ta | 3 | no cis | 2 | 66 | 66 | 77 | 1 | M | 8′ |
| Tumor | 1415-1 | Ta | 2 | no cis | 2 | 28 | 25 | 31 | 1 | M | 65 |
| Tumor | 1828-1 | Ta | 3 | no CIS | 2 | 7 | 7 | 7 | 0 | M | 78 |
| Tumor | 217-11 | Ta | 2 | no cis | 2 | 61 | 61 | 71 | 0 | M | 5′ |
| Tumor | 242-3 | Ta | 3 | CIS | 2 | 12 | 12 | 15 | 1 | M | 74 |
| Tumor | 646-1 | Ta | 2 | no CIS | 2 | 11 | 4 | 11 | 1 | F | 72 |
| Tumor | 856-2 | Ta | 2 | CIS | 2 | 47 | 1 | 47 | 1 | M | 75 |
| Tumor | 992-1 | Ta | 2 | no cis | 2 | 20 | 18 | 21 | 1 | M | 67 |
| Tumor | 1327-1 | Ta | 2 | no cis | 1 | 22 | 22 | 26 | 2 | F | 67 |
| Tumor | 1354-1 | Ta | 3 | no cis | 1 | 12 | 23 | 12 | 1 | M | 80 |
| Tumor | 1656-1 | Ta | 3 | no cis | 1 | 12 | 12 | 15 | 0 | F | 80 |
| Tumor | 1066-1 | Ta | 3 | no cis | 0 | 67 | 67 | 69 | 0 | M | 80 |
| Tumor | 1077-1 | Ta | 2 | no cis | 0 | 62 | 62 | 63 | 0 | M | 66 |
| Tumor | 1105-1 | Ta | 2 | no cis | 0 | 31 | 31 | 53 | 0 | M | 57 |
| Tumor | 1335-1 | Ta | 2 | CIS | 0 | 42 | 42 | 43 | 0 | M | 48 |
| Tumor | 1350-1 | Ta | 3 | no cis | 0 | 54 | 54 | 54 | 0 | M | 84 |
| Tumor | 1352-1 | Ta | 2 | no cis | 0 | 38 | 38 | 41 | 0 | F | 43 |
| Tumor | 1408-1 | Ta | 1 | no cis | 0 | 36 | 36 | 38 | 0 | M | 33 |
| Tumor | 332-1 | Ta | 2 | no cis | 0 | 66 | 66 | 116 | 0 | M | 69 |
| Tumor | 521-1 | Ta | 1 | no cis | 0 | 45 | 46 | 105 | 0 | M | 27 |
| Tumor | 62-9 | Ta | 3 | CIS | 0 | 44 | 44 | 52 | 3 | M | 70 |
| Tumor | 669-1 | Ta | 2 | no cis | 0 | 95 | 95 | 95 | 0 | M | 59 |
| Tumor | 686-1 | Ta | 2 | no cis | 0 | 81 | 81 | 81 | 0 | M | 74 |
| Tumor | 833-2 | Ta | 3 | no cis | 0 | 73 | 73 | 73 | 0 | F | 44 |
| Tumor | 876-1 | Ta | 2 | no cis | 0 | 85 | 85 | 87 | 0 | M | 57 |
| Tumor | 997-1 | Ta | 2 | no cis | 0 | 53 | 53 | 69 | 0 | M | 52 |
| Tumor | 1017-1 | T1 | 3 | CIS | 2 | 42 | 9 | 52 | 1 | M | 79 |
| Tumor | 1047-1 | T1 | 3 | CIS | 2 | 14 | 6 | 17 | 1 | M | 76 |
| Tumor | 1056-1 | T1 | 3 | CIS | 2 | 51 | 25 | 60 | 1 | F | 58 |
| Tumor | 1082-1 | T1 | 3 | CIS | 2 | 69 | 69 | 76 | 0 | F | 81 |
| Tumor | 1134-1 | T1 | 3 | no cis | 2 | 28 | 25 | 43 | 1 | M | 81 |
| Tumor | 1224-1 | T1 | 3 | CIS | 2 | 25 | 11 | 26 | 1 | F | 73 |
| Tumor | 1252-1 | T1 | 3 | CIS | 2 | 58 | 58 | 60 | 1 | M | 65 |
| Tumor | 1336-1 | T1 | 3 | no cis | 2 | 54 | 40 | 58 | 0 | M | 77 |
| Tumor | 1425-1 | T1 | 3 | ND | 2 | 26 | 2 | 26 | 0 | M | 70 |
| Tumor | 1443-1 | T1 | 3 | ND | 2 | 10 | 10 | 53 | 0 | M | 69 |
| Tumor | 1456-1 | T1 | 3 | no cis | 2 | 19 | 19 | 32 | 1 | F | 72 |
| Tumor | 1571-1 | T1 | 3 | CIS | 2 | 2 | 2 | 9 | 1 | M | 68 |
| Tumor | 1660-1 | T1 | 3 | ND | 2 | 2 | 2 | 8 | 0 | M | 80 |
| Tumor | 1887-1 | T1 | 3 | no cis | 2 | 2 | 1 | 11 | 0 | F | 67 |
| Tumor | 247-9 | T1 | 3 | CIS | 2 | 15 | 15 | 18 | 1 | M | 81 |
| Tumor | 320-7 | T1 | 3 | CIS | 2 | 8 | 3 | 10 | 1 | M | 69 |
| Tumor | 355-1 | T1 | 3 | no CIS | 2 | 17 | 7 | 17 | 1 | M | 59 |
| Tumor | 886-1 | T1 | 3 | no cis | 2 | 5 | 5 | 10 | 1 | M | 76 |
| Tumor | 938-1 | T1 | 3 | ND | 2 | 0 | 7 | 7 | 1 | M | 83 |
| Tumor | 1725-1 | T1 | 3 | no cis | 2 | 4 | 4 | 4 | 3 | M | 62 |
| Tumor | 1010-1 | T1 | 3 | CIS | 0 | 77 | 77 | 82 | 0 | F | 69 |
| Tumor | 1031-1 | T1 | 3 | CIS | 0 | 70 | 70 | 81 | 0 | M | 68 |
| Tumor | 1034-2 | T1 | 3 | CIS | 0 | 70 | 70 | 79 | 0 | F | 74 |
| Tumor | 1065-1 | T1 | 3 | no cis | 0 | 68 | 68 | 79 | 0 | M | 83 |
| Tumor | 1073-1 | T1 | 3 | no cis | 0 | 72 | 72 | 76 | 3 | F | 65 |
| Tumor | 1182-1 | T1 | 3 | CIS | 0 | 64 | 64 | 68 | 0 | M | 45 |
| Tumor | 1280-1 | T1 | 3 | no cis | 0 | 47 | 47 | 62 | 0 | M | 62 |
| Tumor | 1293-1 | T1 | 3 | no cis | 0 | 45 | 45 | 61 | 0 | M | 64 |
| Tumor | 1375-1 | T1 | 2 | no cis | 0 | 39 | 39 | 52 | 0 | M | 53 |
| Tumor | 1482-1 | T1 | 3 | no cis | 0 | 37 | 37 | 41 | 0 | M | 67 |
| Tumor | 177-1 | T1 | 3 | no cis | 0 | 67 | 67 | 133 | 0 | M | 66 |
| Tumor | 684-4 | T1 | 3 | CIS | 0 | 73 | 73 | 81 | 0 | M | 77 |
| Tumor | 735-1 | T1 | 3 | no cis | 0 | 95 | 95 | 95 | 0 | M | 69 |
| Tumor | 760-1 | T1 | 3 | CIS | 0 | 90 | 90 | 101 | 0 | M | 72 |
| Tumor | 645-1 | T1 | 2 | no cis | 0 | 62 | 62 | 62 | 0 | F | 78 |
| Tumor | 855-1 | T1 | 3 | no cis | 0 | 56 | 56 | 56 | 0 | M | 64 |
| Tumor | 681-1 | T1 | 3 | no cis | 0 | 83 | 83 | 83 | 0 | M | 70 |
| Tumor | 927-1 | T1 | 3 | no cis | 0 | 87 | 87 | 88 | 0 | M | 71 |
| Tumor | 998-1 | T1 | 3 | no cis | 0 | 57 | 57 | 81 | 0 | M | 53 |
| Tumor | 1022-1 | T1 | 3 | no cis | 0 | 56 | 56 | 80 | 0 | M | 6′ |
| Tumor | 1574-1 | T2-4 | 3 | — | — | — | — | 5 | 1 | F | 72 |
| Tumor | 1015-1 | T2-4 | 3 | — | — | — | — | 30 | 1 | M | 58 |
| Tumor | 1041-1 | T2-4 | 3 | — | — | — | — | 357 | 0 | M | 62 |
| Tumor | 1044-1 | T2-4 | 3 | — | — | — | — | 38 | 1 | M | 55 |
| Tumor | 1055-1 | T2-4 | 3 | — | — | — | — | 350 | 0 | M | 70 |
| Tumor | 1113-5 | T2-4 | 2 | — | — | — | — | 160 | 0 | F | 62 |
| Tumor | 1154-1 | T2-4 | 2 | — | — | — | — | 36 | 1 | M | 60 |
| Tumor | 1167-1 | T2-4 | 4 | — | — | — | — | 95 | 1 | F | 51 |
| Tumor | 1178-1 | T2-4 | 5 | — | — | — | — | 28 | 1 | M | 68 |
| Tumor | 1271-1 | T2-4 | 3 | — | — | — | — | 47 | 1 | F | 50 |
| Tumor | 1285-1 | T2-4 | 3 | — | — | — | — | 276 | 0 | M | 49 |
| Tumor | 1321-1 | T2-4 | ? | — | — | — | — | 33 | 1 | F | 51 |
| Tumor | 1385-1 | T2-4 | 2 | — | — | — | — | 243 | 1 | M | 67 |
| Tumor | 1485-1 | T2-4 | 4 | — | — | — | — | 43 | 1 | M | 65 |
| Tumor | 1530-1 | T2-4 | 4 | — | — | — | — | 37 | 1 | M | 74 |
| Tumor | 1533-1 | T2-4 | 3 | — | — | — | — | 31 | 1 | M | 56 |
| Tumor | 1682-1 | T2-4 | 3 | — | — | — | — | 42 | 1 | F | 66 |
| Tumor | 217-6 | T2-4 | 2 | — | — | — | — | 36 | 1 | F | 51 |
| Tumor | 472-1 | T2-4 | 3 | — | — | — | — | 14 | 1 | F | 63 |
| Tumor | 523-1 | T2-4 | 3 | — | — | — | — | 57 | 1 | M | 66 |
| Tumor | 575-1 | T2-4 | 3 | — | — | — | — | 83 | 1 | M | 53 |
| Tumor | 621-5 | T2-4 | 2 | — | — | — | — | 76 | 1 | M | 59 |
| Tumor | 724-1 | T2-4 | 3 | — | — | — | — | 420 | 0 | M | 53 |
| Tumor | 752-3 | T2-4 | 3 | — | — | — | — | 190 | 1 | M | 51 |
| Tumor | 805-1 | T2-4 | 3 | — | — | — | — | 62 | 1 | M | 73 |
| Tumor | 882-1 | T2-4 | 4 | — | — | — | — | 77 | 1 | F | 55 |
| Tumor | 981-1 | T2-4 | 3 | — | — | — | — | 28 | 1 | M | 63 |

*Months from tumor to last visit to the clinic, or to cystectomy.

1-110. (canceled)

111. A method for determining the likelihood of bladder cancer progression, comprising determining the expression level of hsa-miR-145 in a sample containing bladder cancer cells from an individual with bladder cancer and comparing it to a standard miRNA expression level in a control tissue, wherein higher expression of hsa-miR-145 in said individual with bladder cancer correlates with a higher risk of progression.

112. The method of claim 111 wherein said control tissue comprises tissue from a representative individual or pool of individuals with bladder cancer wherein said bladder cancer has not progressed.

113. The method of claim 112 wherein progression is one or more of: advancement of the tumor from stage Ta or T1 to stage T2 to T4; formation of invasive bladder cancer tumors and/or recurrence or metastasis within five years of initial diagnosis of bladder cancer; presence of carcinoma-in-situ; or death in a subject from the bladder cancer within a fixed period of time.

114. The method of claim 112 wherein the standard miRNA expression level is from said representative pool of individuals and is a mean, median or other statistically manipulated or otherwise summarized or aggregated representative miRNA expression level for the miRNA level in said control tissues in said individuals.

115. The method of claim 112 wherein the expression level of hsa-miR-518c* is also measured relative to the expression level in said control tissue, and wherein an increased expression level of hsa-miR-518c* correlates with a higher risk of progression.

116. The method of claim 112 wherein the expression level of hsa-miR-133b is also measured relative to the expression level in said control tissue, and wherein an increased expression level of hsa-miR-133b correlates with a higher risk of progression.

117. The method of claim 112 wherein the expression level of hsa-miR-373* is also measured relative to the expression level in said control tissue, and wherein an increased expression level of hsa-miR-373* correlates with a higher risk of progression.

118. The method of claim 115 wherein the expression level of hsa-miR-373* is also measured relative to the expression level in said control tissue, and wherein an increased expression level of hsa-miR-373* correlates with a higher risk of progression.

119. The method of claim 115 wherein the expression level of hsa-miR-133b is also measured relative to the expression level in said control tissue, and wherein an increased expression level of hsa-miR-133b correlates with a higher risk of progression.

120. The method of claim 116 wherein the expression level of hsa-miR-129 is also measured relative to the expression level in said control tissue, and wherein an increased expression level of hsa-miR-129 correlates with a higher risk of progression.

121. The method of claim 116 wherein the expression level of hsa-miR-373* is also measured relative to the expression level in said control tissue, and wherein an increased expression level of hsa-miR-373* correlates with a higher risk of progression.

122. The method of claim 119 wherein the expression levels of hsa-miR-129 and hsa-miR-373* are also measured relative to the expression level in said control tissue, and wherein an increased expression level of hsa-miR-129 and/or hsa-miR-373*correlates with a higher risk of progression.

123. The method of claim 112 wherein the expression level of hsa-miR-29b is also measured relative to the expression level in said control tissue, and wherein a decreased expression level of miR-29b correlates with a higher risk of progression.

124. The method of claim 112 wherein the expression level of hsa-miR-203 is also measured relative to the expression level in said control tissue, and wherein a decreased expression level of hsa-miR-203 correlates with a higher risk of progression.

125. The method of claim 112 wherein the expression level of hsa-miR-205 is also measured relative to the expression level in said control tissue, and wherein a decreased expression level of hsa-miR-205 correlates with a higher risk of progression.

126. The method of claim 112 wherein the expression level of hsa-miR-29a is also measured relative to the expression level in said control tissue, and wherein a decreased expression level of hsa-miR-29a correlates with a higher risk of progression.

127. The method of claim 123 wherein the expression level of hsa-miR-203 is also measured relative to the expression level in said control tissue, and wherein a decreased expression level of hsa-miR-203 correlates with a higher risk of progression.

128. The method of claim 124 wherein the expression level of hsa-miR-205 is also measured relative to the expression level in said control tissue, and wherein a decreased expression level of hsa-miR-205 correlates with a higher risk of progression.

129. The method of claim 124 wherein the expression level of hsa-miR-29a is also measured relative to the expression level in said control tissue, and wherein a decreased expression level of hsa-miR-29a correlates with a higher risk of progression.

130. The method of claim 124 wherein the expression levels of hsa-miR-29b, hsa-miR-205 and hsa-miR-29a are also measured relative to the expression level in said control tissue, and wherein a decreased expression level of any of hsa-miR-29b, hsa-miR-205 and hsa-miR-29a correlates with a higher risk of progression.

131. The method of claim 111 wherein lower expression of hsa-miR-145 in said individual with bladder cancer correlates with a lower risk of progression.

132. The method of claim 115 wherein a decreased expression level of hsa-miR-145 or hsa-miR-518c* correlates with a lower risk of progression.

133. The method of claim 116 wherein a decreased expression level of hsa-miR-145 or hsa-miR-133b correlates with a lower risk of progression.

134. The method of claim 117 wherein a decreased expression level of hsa-miR-145 or hsa-miR-373* correlates with a lower risk of progression.

135. The method of claim 118 wherein a decreased expression level of hsa-miR-145, hsa-miR-518c* or hsa-miR-373* correlates with a lower risk of progression.

136. The method of claim 119 wherein a decreased expression level of hsa-miR-145, hsa-miR-518c* or hsa-miR-133b correlates with a lower risk of progression.

137. The method of claim 120 wherein a decreased expression level of hsa-miR-145, hsa-miR-133b or hsa-miR-129 correlates with a lower risk of progression.

138. The method of claim 121 wherein a decreased expression level of hsa-miR-145, hsa-miR-133b or hsa-miR-373* correlates with a lower risk of progression.

139. The method of claim 122 wherein a decreased expression level of hsa-miR-145, hsa-miR-518c*, hsa-miR-133b, hsa-miR-129 or hsa-miR-373* correlates with a lower risk of progression.

140. The method of claim 123 wherein an increased expression level of hsa-miR-29b correlates with a lower risk of progression.

141. The method of claim 124 wherein an increased expression level of hsa-miR-203 correlates with a lower risk of progression.

142. The method of claim 125 wherein an increased expression level of hsa-miR-205 correlates with a lower risk of progression.

143. The method of claim 126 wherein an increased expression level of hsa-miR-29a correlates with a lower risk of progression.

144. The method of claim 127 wherein an increased expression level of hsa-miR-29b or hsa-miR-203 correlates with a lower risk of progression.

145. The method of claim 128 wherein an increased expression level of hsa-miR-203 or hsa-miR-205 correlates with a lower risk of progression.

146. The method of claim 129 wherein an increased expression level of hsa-miR-203 or hsa-miR-29a correlates with a lower risk of progression.

147. The method of claim 130 wherein an increased expression level of hsa-miR-203, hsa-miR-29a, hsa-miR-29b, hsa-miR-205 or hsa-miR-29a correlates with a lower risk of progression.
